(19) United States
(12) Patent Application Publication
(10) Pub. No.: US 2007/0249590 A1
(43) Pub. Date: Oct. 25, 2007

(54) Substituted indolo[2,3-a]pyrrolo[3,4-c]carbazole compounds useful in treating kinase disorders

(76) Inventors: Lawrence J. Wilson, Atlanta, GA (US); William V. Murray, Belle Mead, NJ (US); Shyh-Ming Yang, High Bridge, NJ (US); Cangming Yang, Highland Park, NJ (US); Bingbing Wang, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/586,817

(22) Filed: Oct. 26, 2006

Related U.S. Application Data
(60) Provisional application No. 60/731,296, filed on Oct. 28, 2005.

(51) Int. Cl. A61K 31/551; A61P 11/06; A61P 17/06; A61P 19/00; A61P 19/02; A61P 37/06; A61P 37/08; A61P 43/00; C07D 243/10; C07D 487/22

(52) U.S. Cl. 514/219; 540/492; 540/555

(57) ABSTRACT

The present invention is directed to substituted indolo[2,3-a]pyrrolo[3,4-c]carbazole compounds of formula (I): 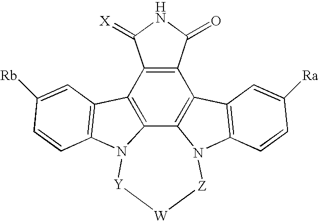 and forms thereof and their synthesis and use as protein kinase inhibitors and interactions thereof.

### CROSS-REFERENCE TO RELATED APPLICATIONS

This present application claims benefit of U.S. Provisional Patent Application Ser. No. 60/731,296, filed Oct. 28, 2005, which is incorporated herein by reference in its entirety and for all purposes.

### FIELD OF THE INVENTION

The present invention relates to a series of substituted indolo[2,3-a]pyrrolo[3,4-c]carbazole compounds, pharmaceutical compositions and methods for use thereof. In particular, the substituted indolo[2,3-a]pyrrolo[3,4-c]carbazole compounds of the present invention are protein kinase inhibitors useful in preventing, treating or ameliorating a kinase mediated disorder.

### BACKGROUND OF THE INVENTION

In general, protein kinases are the largest set of structurally related phosphoryl transferases, have highly conserved structures and catalytic functions and may be categorized into families by the substrates they phosphorylate (e.g., protein-tyrosine, protein-serine/threonine, histidine and the like) and are responsible for the control of a wide variety of cellular signal transduction processes.

Examples of protein-tyrosine kinases include, but are not limited to, Irk, IGFR-1, Zap-70, Bmx, Btk, CHK (Csk homologous kinase), CSK (C-terminal Src Kinase), Itk-1, Src (c-Src, Lyn, Fyn, Lck, Syk, Hck, Yes, Blk, Fgr and Frk), Tec, Txk/R1k, Abl, EGFR (EGFR-1/ErbB-1, ErbB-2/NEU/HER-2, ErbB-3 and ErbB-4), FAK, FGF1R (also FGFR1 or FGR-1), FGF2R (also FGR-2), MET (also Met-1 or c-MET), PDGFR-α, PDGFR-β, Tie-1, Tie-2 (also Tek-1 or Tek), VEGFR1 (also FLT-1), VEGFR2 (also KDR), FLT-3, FLT-4, c-KIT, JAK1, JAK2, JAK3, TYK2, LOK, RET, TRKA, PYK2, ALK (Anaplastic Lymphoma Kinase), EPHA (1-8), EPHB (1-6), RON, Fes, Fer or EPHB4 (also EPHB4-1).

Examples of protein-serine/threonine kinases include, but are not limited to, Ark, ATM (1-3), CamK (I-IV), CamKK, Chk1 and 2 (Checkpoint kinases), CK1, CK2, Erk, IKK-I (also IKK-ALPHA or CHUK), IKK-2 (also IKK-BETA), Ilk, Jnk (1-3), LimK (1 and 2), MLK3Raf (A, B, and C), CDK (1-10), PKC (including all PKC subtypes), Plk (1-3), NIK, Pak (1-3), PDK1, PKR, RhoK, RIP, RIP-2, GSK3 (A and B), PKA, P38, Erk (1-3), PKB (including all PKB subtypes) (also AKT-1, AKT-2, AKT-3 or AKT3-r), IRAK1, FRK, SGK, TAK1 or Tpl-2 (also COT).

Protein kinases play very important roles in the normal regulation of cell growth. However, as a result of either mutation or overexpression of the tyrosine kinases (receptor or non-receptor) or the ligands of the receptor tyrosine kinases, signaling can become deregulated, resulting in uncontrolled cell proliferation leading to cancer or a related disease, disorder or syndrome.

The process of phosphorylation is catalyzed and regulated by protein kinases, whereby the kinases covalently attach phosphate groups to proteins or lipid targets in response to a variety of extracellular signals: hormones, neurotransmitters, growth and differentiation factors, cell cycle events, environmental stresses, nutritional stresses and the like.

In turn, phosphorylation modulates or regulates a variety of cellular processes such as proliferation, growth, differentiation, metabolism, apoptosis, motility, transcription, translation and other signaling processes. Defective control of protein phosphorylation has also been implicated in a number of diseases and disease conditions. Accordingly, kinase inhibitors have potential use as therapeutic agents.

The tyrosine kinases are categorized by whether they are receptor tyrosine kinases or non-receptor tyrosine kinases. The receptor tyrosine kinases span the cell membrane with a ligand interacting domain protruding from the cell, with a hydrophobic trans-membrane domain, and a cytoplasmic domain that contains the catalytic kinase domain and other regulatory sequences. Non-receptor tyrosine kinases are often myristylated or modified by the addition of other hydrophobic moieties that allow them to be anchored to the cell membrane.

Due to the lack of intrinsic kinase activity associated with cytokine receptors, cells expressing cytokine receptors depend on non-receptor tyrosine kinases for inducing biological responses.

The Janus (JAK) protein tyrosine kinase (PTK) families are cytoplasmic non-receptor protein tyrosine kinases that play a pivotal role in cytokine signal transduction pathways through association with various cytokine receptors. The members of the JAK family include JAK1, JAK2, JAK3 and Tyk2. The JAK family does not exhibit a Src kinase-like SH2 and SH3 signaling domain, but contains a distinct JH (JH1 and JH2) domain for signaling.

The basic prototype of the JAK-dependent signal transduction pathway begins with cytokine binding to transmembrane receptors, which in turn leads to activation of the JAK kinase family. The activated receptor-kinase complexes recruit members of the STAT (Signal Transducers and Activators of Transcription) family, which become activated upon phosphorylation by JAK.

As a consequence, the phosphorylated STAT proteins dimerize and translocate to the nucleus. In the nucleus, STAT complexes bind response elements in the promoters of target genes and stimulate transcription of these genes. Since different ligands employ specific JAK family members, utilization of this pathway mandates specificity in signaling cascades and contributes to a diverse array of cellular responses.

Cytokines control many biological processes, but are especially important for regulating inflammatory and immune responses.

JAK3 is a key member of JAK family and was identified by three independent groups in 1994. JAK3 is highly restricted to hematopoietic cells, unlike other members of the JAK family that are expressed ubiquitously. Unlike the other members of the JAK family, which are widely expressed and bind to several cytokine receptors, JAK3 has limited tissue distribution and seems to interact uniquely with the common γ-subunit (γc) for the receptor of six specific interleukin cytokines: interleukin-2 (IL-2), IL-4, IL-7, IL-9, IL-15 and IL-21, thus inducing the signaling response.

The interleukin cytokines selectively activate JAK3 because JAK3 selectively binds to the γc chain of the receptors. The interleukins play a crucial role in lymphoid development and function and are associated with many of the basic functions of normal immunity, including foreign pathogen recognition and self tolerance. The IL-2 cytokine plays a critical role in helper and memory T-cell development. Human genetic abnormalities, where either the absence of the JAK3 enzyme or the γc subunit have been identified, are associated with rare and inherited defects in primary immunity known as SCID (severe combined immunodeficiency).

The genomic structure and mapping of JAK3 has been determined. The mapping analysis of JAK3 shows that the kinase is encoded by a 4.3 kB mRNA in humans and maps to the human chromosome 19p12-13.1. A cluster of genes, proto-oncogenes and transcription factors are also located near this region.

The physiological role of JAK3 has been borne out through studies with JAK3 knockout mice that were generated by targeted disruption of the JAK3 gene in embryonic stem cells and through the genetic analysis of patients with severe combined immunodeficiency (SCID). Although the deficiency of JAK3 in humans typically results in the lack of T cells and NK cell development, the development of B cells is not affected. JAK3 knockout mice that were generated by targeted disruption of the JAK3 gene exhibited profound immunological defects. Unlike humans, these mice show lack of B cells and have relatively small numbers of T cells. JAK3-knockout mice showed no detectable defects in the development of myeloid lineage. Although non lymphoid cells such as monocytes, megakaryocytes, and endothelial cells also express JAK3, to the exclusion of the non-lymphoid immune system, JAK3 appears to play a key role in the development of the lymphoid immune system.

The initial belief was that a primary function of JAK3 was to regulate proliferation of T and B cells through a cytokine dependent pathway. Recent studies, however, have shown that JAK3 can also transduce signals in non-cytokine dependent biological responses. For example, mast cells have been shown to express JAK3 and that the enzymatic activity of JAK3 is enhanced by IgE receptor crosslinking. Studies with JAK3-knockout mice and JAK3 specific inhibitors have shown that JAK3 plays a key role in mast cell mediated inflammatory responses.

Therefore a JAK3 antagonist in a normal functioning immune system would be useful and effective as an immunosuppressant, finding uses in the many autoimmune based disease states such as, but not limited to, transplantation rejection, psoriasis, psoriatic arthritis, graft-versus-host disease, multiple sclerosis, inflammatory bowel disease, systemic lupus erythematosus, rheumatoid arthritis, allergic diseases and asthma.

Bisindole and staurosporine-like compounds have been disclosed in U.S. Pat. Nos. 5,438,050; 5,883,114; 5,945,440 (all from Kleinschroth et al.), 5,705,511 (Hudkins et al.) and 6,013,646 (Roder et al.) and in PCT applications WO8807045, WO00130151 WO0016781 and WO0230941.

### SUMMARY OF THE INVENTION

The present invention is directed to compounds of formula (I): 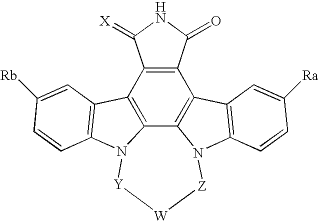
and forms thereof, wherein Ra, Rb, W, X, Y and Z are as defined herein.

An example of the present invention includes a method for using a compound of formula (I) as a protein kinase inhibitor, such as a JAK inhibitor, for preventing, treating or ameliorating a kinase mediated disease, disorder or condition in a subject in need thereof comprising administering to the subject an effective amount of a compound of formula (I) or composition thereof.

The present invention is also directed to a method for preventing, treating or ameliorating a kinase mediated disease, disorder or condition in a subject in need thereof comprising administering to the subject an effective amount of a compound of formula (I) or composition thereof.

### DETAILED DESCRIPTION OF THE INVENTION

The present invention provides a compound of formula (I) 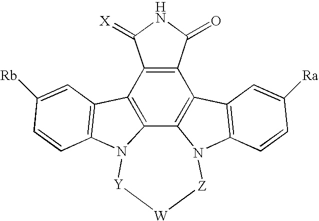
or a form thereof, wherein
X is H, H or O;
Y and Z is each methyl or ethyl;
W is —C(R₁,R₁ₐ)—C(R₂,R₂ₐ)—, —C(R₃)═C(R₄)—, —C(R₅,R₅ₐ)—, —C(R₆)—, —O—, R₇-heterocyclyl, R₇—C₃₋₈ cycloalkyl, R₇-heteroaryl or R₇-aryl;
R₁, R₁ₐ, R₂, R₂ₐ, R₅ and R₅ₐ is each selected from R₇, C₁₋₈ alkyl-carbamoyl, carbamoyloxy, carbamoyloxy-C₁₋₈alkyl C₁₋₈alkyl-carbamoyloxy, C₁₋₈alkyl-carbamoyloxy-C₁₋₈alkyl, R₇-heterocyclyl-carbamoyl, heterocyclyl-carbonyl, carbonyloxy, heterocyclyl-carbonyloxy or heterocyclyl-carbonyloxy-C₁₋₈alkyl,
wherein when R₁, R₁ₐ, R₂ and R₂ₐ is each selected from R₇, then no more than three of R₁, R₁ₐ, R₂ and R₂ₐ are hydrogen,
wherein when R₅ and R₅ₐ is each selected from R₇, then no more than one of R₅ and R₅ₐ are hydrogen,
wherein C₁₋₈alkyl-carbamoyl is optionally substituted on C₁₋₈alkyl with one, two or three substituents each selected from C₁₋₈ alkoxy, C₁₋₈ alkoxycarbonyl, amino, C₁₋₈alkyl-amino, halogen, hydroxy, R₇-heterocyclyl, R₇—C₃₋₈ cycloalkyl, R₇-heteroaryl or R₇-aryl,
wherein carbamoyloxy and carbamoyloxy-C₁₋₈alkyl is each substituted on nitrogen with one substituent selected from hydrogen or C₁₋₈alkyl and one other substituent selected from R₇-heterocyclyl or R₇-aryl-C₁₋₈alkyl-heterocyclyl,
wherein C₁₋₈alkyl-carbamoyloxy is optionally substituted on C₁₋₈alkyl with one, two or three substituents each selected from C₁₋₈ alkoxy, C₁₋₈ alkoxycarbonyl, amino, C₁₋₈ alkyl-amino, halogen, hydroxy, R₇-heterocyclyl, R₇—C₃₋₈ cycloalkyl, R₇-heteroaryl or R₇-aryl,
wherein C₁₋₈ alkyl-carbamoyloxy-C₁₋₈ alkyl is optionally substituted on C₁₋₈ alkyl with one, two or three substituents each selected from C₁₋₈ alkoxy, C₁₋₈ alkoxycarbonyl, amino, C₁₋₈ alkyl-amino, halogen, hydroxy, R₇-heterocyclyl, R₇—C₃₋₈ cycloalkyl, R₇-heteroaryl or R₇-aryl,
wherein heterocyclyl-carbonyl is substituted on heterocyclyl with one or two substituents each selected from R₇, R₇—C₃₋₈ cycloalkyl, R₇-aryl, R₇-aryl-C₁₋₈ alkyl, R₇-heteroaryl, R₇-heteroaryl-C₁₋₈ alkyl, R₇-heterocyclyl-C₁₋₈ alkyl or R₇-heterocyclyl-carbonyl-C₁₋₈ alkyl,
wherein carbonyloxy is substituted on-carbonyl with C₁₋₈ alkyl, C₁₋₈ alkoxy-C₁₋₈ alkyl or C₁₋₈ alkyl-amino-C₁₋₈ alkyl,
wherein heterocyclyl-carbonyloxy is substituted on heterocyclyl with one or two substituents each selected from R₇, R₇—C₃₋₈ cycloalkyl, R₇-aryl, R₇-aryl-C₁₋₈ alkyl, (R₇-aryl)₂-C₁₋₈ alkyl, R₇-heteroaryl, R₇-heteroaryl-C₁₋₈ alkyl, R₇-heterocyclyl, R₇-heterocyclyl-C₁₋₈ alkyl or R₇-heterocyclyl-C₁₋₈ acyl, and
wherein heterocyclyl-carbonyloxy-C₁₋₈alkyl is substituted on heterocyclyl with one or two substituents each selected from R₇, R₇—C₃₋₈ cycloalkyl, R₇-aryl, R₇-aryl-C₁₋₈ alkyl, (R₇-aryl)₂-C₁₋₈ alkyl, R₇-aryl-C₁₋₈ alkoxycarbonyl, R₇-heteroaryl, R₇-heteroaryl-C₁₋₈ alkyl, R₇-heterocyclyl, R₇-heterocyclyl-C₁₋₈alkyl or R₇-heterocyclyl-C₁₋₈ acyl,
alternatively, R₅ and R₅ₐ are taken together with the carbon atom of attachment to form a ring system selected from R₇-heterocyclyl, R₇—C₃₋₈ cycloalkyl, R₇-heteroaryl or R₇-aryl, wherein the carbon atom of attachment is a member atom of the ring system;
R₃ and R₄ is each selected from hydrogen, C₁₋₈ alkyl, C₁₋₈ acyl or C₁₋₈ alkoxycarbonyl;
R₆ is selected from C₁₋₈ alkylene substituted with one, two or three substituents each selected from C₁₋₈ alkoxy, C₁₋₈ alkoxycarbonyl, amino, C₁₋₈ alkyl-amino, halogen or hydroxy;
R₇ is one, two, three, four or five substituents each selected from hydrogen, C₁₋₈ alkyl, C₁₋₈ alkoxy, C₁₋₈ acyl, amino, C₁₋₈ alkyl-amino, C₁₋₈ alkyl-amino-C₁₋₈ alkyl, carboxy, C₁₋₈ alkoxycarbonyl, C₁₋₈ alkoxy-amido, halogen, hydroxy, oxo, halo-C₁₋₈ alkyl, halo-C₁₋₈ alkoxy, hydroxy-C₁₋₈ alkyl, hydroxy-C₁₋₈ alkoxy, hydroxy-C₁₋₈ alkoxy-C₁₋₈ alkyl or aminosulfonyl;
Ra and Rb is each selected from R₈, amino-C₁₋₈ alkyl, thio-C₁₋₈ alkyl, imino-C₁₋₈ alkyl, carbamoyl, C₁₋₈ alkyl-carbamoyl, C₁₋₈ alkyl-carbamoyl-C₂₋₈ alkenyl, amino-C₁₋₈ alkyl-carbamoyl-C₂₋₈ alkenyl, C₁₋₈ alkyl-amino-C₁₋₈ alkyl-carbamoyl-C₂₋₈ alkenyl, R₈-heterocyclyl, R₈-heterocyclyl-C₁₋₈ alkyl, R₈-heterocyclyl-C₁₋₈ alkoxy, R₈-heterocyclyl-amino, R₈-heterocyclyl-amino-C₂₋₈ alkenyl, R₈-heterocyclyl-C₁₋₈ acyl-amino, R₈—C₃₋₈ cycloalkyl, R₈—C₃₋₈ cycloalkyl-C₁₋₈ alkyl, R₈-aryl, R₈-aryl-C₁₋₈ alkyl, R₈-heteroaryl, R₈-heteroaryl-C₁₋₈ alkyl or R₉-heteroaryl-C₂₋₈ alkenyl,
wherein amino-C₁₋₈ alkyl is optionally substituted on nitrogen with one or two substituents each selected from C₁₋₈ alkyl, C₁₋₈ alkoxy-C₁₋₈ alkyl, R₈-heterocyclyl, R₈-heterocyclyl-C₁₋₈ alkyl, R₈—C₃₋₈ cycloalkyl-C₁₋₈ alkyl, R₈-aryl-C₁₋₈ alkyl or R₈-heteroaryl-C₁₋₈ alkyl,
wherein thio-C₁₋₈ alkyl is substituted on sulfur with C₁₋₈ alkyl, amino-C₁₋₁₈ alkyl or C₁₋₈ alkyl-amino-C₁₋₈ alkyl, and
wherein imino-C₁₋₈ alkyl is optionally substituted on nitrogen with C₁₋₈ alkyl, C₁₋₈ alkoxy-C₁₋₈ alkyl, R₈-heterocyclyl-amino, R₈-heterocyclyl-C₁₋₈ alkyl, R₈—C₃₋₈ cycloalkyl-C₁₋₈ alkyl, R₈-aryl-C₁₋₈ alkyl, R₈-heteroaryl-amino or R₈-heteroaryl-C₁₋₈ alkyl, and
R₈ is one, two, three or four substituents each selected from hydrogen, C₁₋₈ alkyl, C₁₋₈ alkoxy, C₁₋₈ alkoxy-C₁₋₈ alkyl, C₁₋₈ acyl, C₁₋₈ alkoxycarbonyl, carboxy, carboxy-C₁₋₈ alkyl, carboxy-C₂₋₈ alkenyl, amino, C₁₋₈ alkyl-amino, halogen, hydroxy, oxo, nitro, halo-C₁₋₈ alkyl, halo-C₁₋₈ alkoxy, hydroxy-C₁₋₈ alkyl or hydroxy-C₁₋₈ alkoxy.

An example of the present invention is a compound of formula (I) wherein Y—W-Z, X, Ra and Rb are dependently selected from:

| Cpd | Y-W-Z | X | 3-Ra, 9-Rb |
|---|---|---|---|
| 1 | —CH₂CH═CHCH₂— | O | H |
| 2 | —CH₂CH═CHCH₂— | H₂ | H |
| 3 | —CH₂C(CO₂CH₃)═CHCH₂— | O | H |
| 4 | —CH₂C(CH₃)═C(CH₃)CH₂— | H₂ | H |
| 5 | —(CH₂)₂CH═CH(CH₂)₂— | H₂ | H |
| 6 | —CH₂CH═CHCH₂— | H₂ | 3-Br |
| 7 | —(CH₂)₂CH(OH)CH₂— | O | H |
| 8 | —(CH₂)₃CH(OH)CH₂— | O | H |
| 9 | —CH₂CH(OH)CH(OH)CH₂— | O | H |
| 10 | —CH₂CH(OCH₃)—CH(OCH₃)CH₂— | O | H |
| 11 | —CH₂CH(OH)—CH(OH)(CH₂)₂— | O | H |
| 12 | —CH₂CH(OH)—C[(OH)(CO₂CH₃)]CH₂— | O | H |
| 13 | —CH₂CH(OH)—C[(OH)(CO₂H)]CH₂— | O | H |
| 14 | —CH₂CH(OH)—CH(OH)CH₂— | H₂ | H |
| 15 | —CH₂CH[OC(O)CH₂N(CH₃)₂]—CH[OC(O)CH₂N(CH₃)₂]CH₂— | H₂ | H |
| 16 | —CH₂[(4S,5S)-2,2-(CH₃)₂-[1,3]dioxolan- | H₂ | H |
| | 4,5-yl]CH₂— | | |
| 17 | —CH₂[4R,5R)-2,2-(CH₃)₂-[1,3]dioxolan- | H₂ | H |
| | 4,5-yl]CH₂— | | |
| 18 | —CH₂CH(S—OH)—CH(S—OH)CH₂— | H₂ | H |
| 19 | —CH₂CH(R—OH)—CH(R—OH)CH₂— | H₂ | H |
| 20 | —CH₂C[(OH)(CH₃)]—C[(OH)(CH₃)]CH₂— | H₂ | H |
| 21 | —(CH₂)₂CH(OH)—CH(OH)(CH₂)₂— | H₂ | H |
| 22 | —CH₂C(═CH₂)CH₂— | H₂ | H |
| 23 | —CH₂CH(CH₂OH)CH₂— | H₂ | H |
| 24 | —CH₂C[═C(CH₂OH)₂]CH₂— | H₂ | H |
| 25 | —CH₂C[(OH)(CH₂OH)]CH₂— | H₂ | H |
| 26 | —CH₂C[(5-spiro)-2,2-(CH₃)₂-[1,3]dioxan- | H₂ | H |
| | 5-yl]CH₂— | | |
| 27 | —CH₂C[(CH₂OH)₂]CH₂— | H₂ | H |
| 28 | —(CH₂)₂CH(OH)(CH₂)₂— | H₂ | H |
| 29 | —(CH₂)₂O(CH₂)₂— | H₂ | H |
| 30 | —CH₂(1H-pyrrol-3,4-yl)CH₂— | H₂ | H |
| 31 | —CH₂CH(OH)(CH₂)₂— | O | 3-Br |
| 32 | —CH₂CH(OH)(CH₂)₂— | O | 3-pyridin-3-yl |
| 33 | —CH₂CH(OH)(CH₂)₂— | O | 3-pyridin-4-yl |
| 34 | —CH₂CH(OH)(CH₂)₂— | O | 3-pyrimidin-5-yl |
| 35 | —CH₂CH(OH)(CH₂)₂— | O | 3-pyrazin-2-yl |
| 36 | —CH₂CH[OC(O)NH(CH₂)₃-1H-imidazol- | O | H |
| | 1-yl](CH₂)₂— | | |
| 37 | —CH₂CH[OC(O)NHCH₂—C(O)OC(CH₃)₃](CH₂)₂— | O | H |
| 38 | —CH₂CH[OC(O)NH(CH₂)₂CH₃](CH₂)₂— | O | H |
| 39 | —CH₂CH[OC(O)NHCH(CH₃)₂](CH₂)₂— | O | H |
| 40 | —CH₂CH[OC(O)NHC(CH₃)₃](CH₂)₂— | O | H |
| 41 | —CH₂CH[OC(O)NH(CH₂)₂OCH₃](CH₂)₂— | O | H |
| 42 | —CH₂CH[OC(O)NH(CH₂)₃-morpholin-4- | O | H |
| | yl](CH₂)₂— | | |
| 43 | —CH₂CH[OC(O)NH(CH₂)₃-(4-CH₃- | O | H |
| | piperazin-1-yl)](CH₂)₂— | | |
| 44 | —CH₂CH[OC(O)-(4-benzyl-piperazin-1- | O | H |
| | yl](CH₂)₂— | | |
| 45 | —CH₂CH[OC(O)NH-(4-CH₃- | O | H |
| | benzyl)](CH₂)₂— | | |
| 46 | —CH₂CH[OC(O)NHCH₂- | O | H |
| | benzo[1,3]dioxol-5-yl](CH₂)₂— | | |
| 47 | —CH₂CH[OC(O)NHCH₂-pyridin-4- | O | H |
| | yl](CH₂)₂— | | |
| 48 | —CH₂CH[OC(O)NHCH₂-(5-CH₃-furan-2- | O | H |
| | yl)](CH₂)₂— | | |
| 49 | —CH₂CH{OC(O)NH(CH₂)₂-[3,4-(OCH₃)₂- | O | H |
| | phenyl]}(CH₂)₂— | | |
| 50 | —CH₂CH[OC(O)NH(CH₂)₂—N(CH₃)₂](CH₂)₂— | O | H |
| 51 | —CH₂CH[OC(O)-(4-CH₃-piperazin-1- | O | H |
| | yl)](CH₂)₂— | | |
| 52 | —CH₂CH[OC(O)-(2-CH₂-pyrrolidin-1-yl- | O | H |
| | pyrrolidin-1-yl)](CH₂)₂— | | |
| 53 | —CH₂CH[OC(O)-(4-cyclohexyl-piperazin- | O | H |
| | 1-yl](CH₂)₂— | | |
| 54 | —CH₂CH[OC(O)-(4-CH₂- | O | H |
| | benzo[1,3]dioxol-5-yl-piperazin-1 | | |
| | yl)](CH₂)₂— | | |
| 55 | —CH₂CH[OC(O)-(4-pyridin-4-yl- | O | H |
| | piperazin-1-yl)](CH₂)₂— | | |
| 56 | —CH₂CH{OC(O)-[4-(CH₂)₂-morpholin-4- | O | H |
| | yl-piperazin-1-yl]}(CH₂)₂— | | |
| 57 | —CH₂CH{OC(O)-[4-(CH₂)₂-(2-oxo- | O | H |
| | pyrrolidin-1-yl)-piperazin-1-yl]}(CH₂)₂— | | |
| 58 | —CH₂CH{OC(O)-[4-(4-OH-phenyl)- | O | H |
| | piperazin-1-yl]}(CH₂)₂— | | |
| 59 | —CH₂CH{OC(O)-[4-(4-C(O)CH₃-phenyl)- | O | H |
| | piperazin-1-yl]}(CH₂)₂— | | |
| 60 | —CH₂CH[OC(O)-[1,4]diazepan-1- | O | H |
| | yl](CH₂)₂— | | |
| 61 | —CH₂CH[OC(O)—N(CH₃)-(1-benzyl- | O | H |
| | pyrrolidin-3-yl)](CH₂)₂— | | |
| 62 | —CH₂CH[OC(O)-(4-benzhydryl-piperazin- | O | H |
| | 1-yl)](CH₂)₂— | | |
| 63 | —CH₂CH[OC(O)-(4-pyridin-2-yl- | O | H |
| | piperazin-1-yl)](CH₂)₂— | | |
| 64 | —CH₂CH[OC(O)-(4-phenyl-piperazin-1- | O | H |
| | yl)](CH₂)₂— | | |
| 65 | —CH₂CH{OC(O)-[4-(CH₂)₂-phenyl- | O | H |
| | piperazin-1-yl]}(CH₂)₂— | | |
| 66 | —CH₂CH[OC(O)-(4-(CH₂)₂OH-piperazin- | O | H |
| | 1-yl)](CH₂)₂— | | |
| 67 | —CH₂CH[OC(O)—N(CH₃)(CH₂)₂—N(CH₃)₂](CH₂)₂— | O | H |
| 68 | —CH₂CH{OC(O)-[4-(CH₂)₃N(CH₃)₂- | O | H |
| | piperazin-1-yl]}(CH₂)₂— | | |
| 69 | —CH₂CH[OC(O)—N(benzyl)(CH₂)₂—N(CH₃)₂]CH(OH)CH₂— | O | H |
| 70 | —CH₂CH{OC(O)-[4-(2-OCH₃-phenyl)- | O | H |
| | piperazin-1-yl]}(CH₂)₂— | | |
| 71 | —CH₂CH[OC(O)-morpholin-4-yl](CH₂)₂— | O | H |
| 72 | —CH₂CH[OC(O)NH-pyrrolidin-3- | O | H |
| | yl](CH₂)₂— | | |
| 73 | —CH₂CH{OC(O)—[(3S)-3-N(CH₃)₂- | O | H |
| | pyrrolidin-1-yl]}(CH₂)₂— | | |
| 74 | —CH₂CH{OC(O)—[(3R)-3-N(CH₃)₂- | O | H |
| | pyrrolidin-1-yl]}(CH₂)₂— | | |
| 75 | —CH₂CH[OC(O)NH-piperidin-4- | O | H |
| | yl](CH₂)₂— | | |
| 76 | —CH₂CH[OC(O)-(4-CH₃-piperazin-1- | O | H |
| | yl)]CH(OH)CH₂— | | |
| 77 | —CH₂CH[OC(O)NH—(CH₂)₂N(CH₃)₂]CH(OH)CH₂— | O | H |
| 78 | —CH₂CH[OC(O)NH-(2-OCH₃- | O | H |
| | benzyl)]CH(OH)CH₂— | | |
| 79 | —CH₂CH[OC(O)NH(CH₂)₃-(2-oxo- | O | H |
| | pyrrolidin-1-yl)]CH(OH)CH₂— | | |
| 80 | —CH₂CH[OC(O)NHCH₂- | O | H |
| | benzo[1,3]dioxol-5-yl]CH(OH)CH₂— | | |
| 81 | —CH₂CH[OC(O)NHCH₂- | O | H |
| | cyclohexyl]CH(OH)CH₂— | | |
| 82 | —CH₂CH[OC(O)NH(CH₂)₂-pyridin-2- | O | H |
| | yl]CH(OH)CH₂— | | |
| 83 | —CH₂CH[OC(O)NH—(CH₂)₂OCH₃]CH(OH)CH₂— | O | H |
| 84 | —CH₂CH{OC(O)NH(CH₂)₂-[3,4-(OCH₃)₂- | O | H |
| | phenyl]}CH(OH)CH₂— | | |
| 85 | —CH₂CH[OC(O)NH—CH(CH₃)₂]CH(OH)CH₂— | O | H |
| 86 | —CH₂CH[OC(O)NHCH₂-(5-CH₃-furan-2- | O | H |
| | yl)]CH(OH)CH₂— | | |
| 87 | —CH₂CH[OC(O)NH(CH₂)₂-(5-OCH₃-1H- | O | H |
| | indol-3-yl)]CH(OH)CH₂— | | |
| 88 | —CH₂CH[OC(O)NH(CH₂)₃-morpholin-4- | O | H |
| | yl]CH(OH)CH₂— | | |
| 89 | —CH₂CH[OC(O)NHCH₂-pyridin-4- | O | H |
| | yl]CH(OH)CH₂— | | |
| 90 | —CH₂CH[OC(O)NH(CH₂)₃-(4-CH₃- | O | H |
| | piperazin-1-yl)]CH(OH)CH₂— | | |
| 91 | —CH₂CH[OC(O)NH(CH₂)₃-1H-imidazol- | O | H |
| | 1-yl]CH(OH)CH₂— | | |
| 92 | —CH₂CH[OC(O)NH(CH₂)₂-pyrrolidin-1- | O | H |
| | yl]CH(OH)CH₂— | | |
| 93 | —CH₂CH[OC(O)NH-(4-N(CH₃)₂- | O | H |
| | benzyl)]CH(OH)CH₂— | | |
| 94 | —CH₂CH{OC(O)-[4-(CH₂)₂-morpholin-4- | O | H |
| | yl]-piperazin-1-yl]}CH(OH)CH₂— | | |
| 95 | —CH₂CH(OC(O)-{[4-C(O)CH₂-pyrrolidin- | O | H |
| | 1-yl]-piperazin-1-yl})CH(OH)CH₂— | | |
| 96 | —CH₂CH[OC(O)-(4-pyridin-4-yl- | O | H |
| | piperazin-1-yl)]CH(OH)CH₂— | | |
| 97 | —CH₂CH{OC(O)-[4-(CH₂)₃N(CH₃)₂- | O | H |
| | piperazin-1-yl]}CH(OH)CH₂— | | |
| 98 | —CH₂CH[OC(O)-morpholin-4- | O | H |
| | yl]CH(OH)CH₂— | | |
| 99 | —CH₂CH[OC(O)-piperidin-1- | O | H |
| | yl]CH(OH)CH₂— | | |
| 100 | —CH₂CH{OC(O)-[3-N(CH₃)₂-pyrrolidin- | O | H |
| | 1-yl]{CH(OH)CH₂— | | |
| 101 | —CH₂CH[OC(O)-(4-cyclohexyl-piperazin- | O | H |
| | 1-yl)]CH(OH)CH₂— | | |
| 102 | —CH₂CH[OC(O)-(4-phenyl-piperazin-1- | O | H |
| | yl)]CH(OH)CH₂— | | |
| 103 | —CH₂CH[OC(O)-(4-benzhydryl-piperazin- | O | H |
| | 1-yl)]CH(OH)CH₂— | | |
| 104 | —CH₂CH{OC(O)-[4-(CH₂)₂OH-piperazin- | O | H |
| | 1-yl]}CH(OH)CH₂— | | |
| 105 | —CH₂CH[OC(O)NH(CH₂)₂-(4-SO₂NH₂- | O | H |
| | phenyl)]CH(OH)CH₂— | | |
| 106 | —CH₂CH[OC(O)-(1-benzyl-piperidin-4- | O | H |
| | yl)]CH(OH)CH₂— | | |
| 107 | —CH₂CH[OC(O)N(CH₃)(CH₂)₂—N(CH₃)₂]CH(OH)CH₂— | O | H |
| 108 | —CH₂CH[OC(O)N(CH₃)-(1-CH₃- | O | H |
| | pyrrolidin-3-yl)]CH(OH)CH₂— | | |
| 109 | —CH₂CH{OC(O)—N[(CH₂)₃N(CH₃)₂]₂}CH(OH)CH₂— | O | H |
| 110 | —CH₂CH{OC(O)-[4-(CH₂)₂-phenyl- | O | H |
| | piperazin-1-yl]}CH(OH)CH₂— | | |
| 111 | —CH₂CH[OC(O)-[1,4]diazepan-1- | O | H |
| | yl]CH(OH)CH₂— | | |
| 112 | —CH₂CH[OC(O)-(4-pyridin-2-yl- | O | H |
| | piperazin-1-yl)]CH(OH)CH₂— | | |
| 113 | —CH₂CH[OC(O)NH-piperidin-4- | O | H |
| | yl]CH(OH)CH₂— | | |
| 114 | —CH₂CH[OC(O)NH—(CH₂)₂N(CH₃)₂](CH₂)₃— | O | H |
| 115 | —CH₂CH{OC(O)-[4-(CH₂)₃N(CH₃)₂- | O | H |
| | piperazin-1-yl]}(CH₂)₃— | | |
| 116 | —CH₂CH[OC(O)NH(CH₂)₃-(4-CH₃- | O | H |
| | piperazin-1-yl)](CH₂)₃— | | |
| 117 | —CH₂CH(OH){C[(OH)[C(O)NHCH₂- | O | H |
| | pyridin-4-yl]}CH₂— | | |
| 118 | —CH₂CH(OH){C(OH)[C(O)NH—CH(CH₂OH)₂]}CH₂— | O | H |
| 119 | —CH₂CH(OH){C(OH)[C(O)-(3-N(CH₃)₂- | O | H |
| | pyrrolidin-1-yl)]}CH₂— | | |
| 120 | —CH₂CH(OH){C(OH)[C(O)NH(CH₂)₃- | O | H |
| | morpholin-4-yl]}CH₂— | | |
| 121 | —CH₂CH(OH){C(OH)[C(O)-morpholin-4- | O | H |
| | yl]}CH₂— | | |
| 122 | —CH₂CH(OH){C(OH)[C(O)NH-(2-oxo- | O | H |
| | tetrahydrofuran-3-yl)]}CH₂— | | |
| 123 | —CH₂CH(OH){C(OH)[C(O)NH—CH(CH₃)₂]}CH₂— | O | H |
| 124 | —CH₂CH(OH){C(OH)[C(O)NH—(CH₂)₂OCH₃]}CH₂— | O | H |
| 125 | —CH₂CH(OH){C(OH)[C(O)-(4-CH₃- | O | H |
| | piperazin-1-yl)]}CH₂— | | |
| 126 | —CH₂CH(OH)(C(OH){C(O)-[4- | O | H |
| | (CH₂)₃N(CH₃)₂-piperazin-1-yl]})CH₂— | | |
| 127 | —CH₂CH(OH){C(OH)[C(O)NH(CH₂)₃-(2- | O | H |
| | oxo-pyrrolidin-1-yl)]}CH₂— | | |
| 128 | —CH₂CH(OH){C(OH)[C(O)NH(CH₂)₂- | O | H |
| | thien-2-yl]}CH₂— | | |
| 129 | —CH₂CH(OH)(C(OH){C(O)-[4-(4-OH- | O | H |
| | phenyl)-piperazin-1-yl]})CH₂— | | |
| 130 | —CH₂CH(OH){C(OH)[C(O)-(4- | O | H |
| | (CH₂)₂OH-piperazin-1-yl)]}CH₂— | | |
| 131 | —CH₂CH(OH){C(OH)[C(O)-(4-pyridin-2- | O | H |
| | yl-piperazin-1-yl)]}CH₂— | | |
| 132 | —CH₂CH(OH){C(OH)[C(O)-(4-OH- | O | H |
| | piperidin-1-yl)]}CH₂— | | |
| 133 | —CH₂CH(OH)(C(OH){C(O)-[4-CH₂C(O)- | O | H |
| | pyrrolidin-1-yl]-piperazin-1-yl})CH₂— | | |
| 134 | —CH₂CH(OH)(C(OH){C(O)-[4-(CH₂)₂- | O | H |
| | morpholin-4-yl]-piperazin-1-yl})CH₂— | | |
| 135 | —CH₂{C(OH)[CH₂OC(O)NH—CH(CH₃)₂]}CH₂— | H₂ | H |
| 136 | —CH₂{C(OH)[CH₂OC(O)NH—(CH₂)₂OCH₃]}CH₂— | H₂ | H |
| 137 | —CH₂(C(OH){CH₂OC(O)-[4-(4-OH- | H₂ | H |
| | phenyl)-piperazin-1-yl]})CH₂— | | |
| 138 | —CH₂{C(OH)[CH₂OC(O)-morpholin-4- | H₂ | H |
| | yl]}CH₂— | | |
| 139 | —CH₂{C(OH)[CH₂OC(O)NH(CH₂)₂- | H₂ | H |
| | pyridin-2-yl]}CH₂— | | |
| 140 | —CH₂{C(OH)[CH₂OC(O)NH(CH₂)₃-(2- | H₂ | H |
| | oxo-pyrrolidin-1-yl)]}CH₂— | | |
| 141 | —CH₂{C(OH)[CH₂OC(O)NHCH₂-(5-CH₃- | H₂ | H |
| | furan-2-yl)]}CH₂— | | |
| 142 | —CH₂{C(OH)[CH₂OC(O)-(4-cyclohexyl- | H₂ | H |
| | piperazin-1-yl)]}CH₂— | | |
| 143 | —CH₂{C(OH)[CH₂OC(O)-(4-CH₂OH- | H₂ | H |
| | piperidin-1-yl)]}CH₂— | | |
| 144 | —CH₂{C(OH)[CH₂OC(O)-(4-pyridin-4-yl- | H₂ | H |
| | piperazin-1-yl)]}CH₂— | | |
| 145 | —CH₂{C(OH)[CH₂OC(O)NH-(1-benzyl- | H₂ | H |
| | piperidin-4-yl)]}CH₂— | | |
| 146 | —CH₂{C(OH)[CH₂OC(O)-[1,4]diazepan- | H₂ | H |
| | 1-yl]}CH₂— | | |
| 147 | —CH₂{C(OH)[CH₂OC(O)-1,2,3,4- | H₂ | H |
| | tetrahydro-isoquinolin-2-yl]}CH₂— | | |
| 148 | —CH₂{C(OH)[CH₂OC(O)N(CH₃)—(CH₂)₂N(CH₃)₂]}CH₂— | H₂ | H |
| 149 | —CH₂{C(OH)[CH₂OC(O)-(4-OH- | H₂ | H |
| | piperidin-1-yl)]}CH₂— | | |
| 150 | —CH₂{C(OH)[CH₂OC(O)-(4-pyrrolidin-1- | H₂ | H |
| | yl-piperidin-1-yl)]}CH₂— | | |
| 151 | —CH₂{C(OH)[CH₂OC(O)NH(CH₂)₃-(4- | H₂ | H |
| | CH₃-piperazin-1-yl)]}CH₂— | | |
| 152 | —CH₂{C(OH)[CH₂OC(O)NH(CH₂)₃-1H- | H₂ | H |
| | imidazol-1-yl]}CH₂— | | |
| 153 | —CH₂(C(OH){CH₂OC(O)NH(CH₂)₂-[3,4- | H₂ | H |
| | (OCH₃)₂-phenyl]})CH₂— | | |
| 154 | —CH₂CH[CH₂OC(O)NH—(CH₂)₂OCH₃]CH₂— | H₂ | H |
| 155 | —CH₂CH[CH₂OC(O)NHCH(CH₃)₂]CH₂— | H₂ | H |
| 156 | —CH₂CH[CH₂OC(O)NHCH₂-(5-CH₃- | H₂ | H |
| | furan-2-yl)]CH₂— | | |
| 157 | —CH₂CH[CH₂OC(O)NH(CH₂)₃-(2-oxo- | H₂ | H |
| | pyrrolidin-1-yl)]CH₂— | | |
| 158 | —CH₂CH{CH₂OC(O)-[4-(4-OH-phenyl)- | H₂ | H |
| | piperazin-1-yl]}CH₂— | | |
| 159 | —CH₂CH[CH₂OC(O)-(4-OH-piperidin-1- | H₂ | H |
| | yl)]CH₂— | | |
| 160 | —CH₂CH[CH₂OC(O)NH(CH₂)₂-pyridin-2- | H₂ | H |
| | yl]CH₂— | | |
| 161 | —CH₂CH{CH₂OC(O)NH(CH₂)₂-[3,4- | H₂ | H |
| | (OCH₃)₂-phenyl]}CH₂— | | |
| 162 | —CH₂CH[CH₂OC(O)-(4-pyrrolidin-1-yl- | H₂ | H |
| | piperidin-1-yl)]CH₂— | | |
| 163 | —CH₂CH[CH₂OC(O)NH-(1-benzyl- | H₂ | H |
| | piperidin-4-yl)]CH₂— | | |
| 164 | —CH₂CH{CH₂OC(O)-[4-(CH₂)₂OH- | H₂ | H |
| | piperazin-1-yl]}CH₂— | | |
| 165 | —CH₂CH[CH₂OC(O)NH(CH₂)₃- | H₂ | H |
| | morpholin-4-yl]CH₂— | | |
| 166 | —CH₂CH{CH₂OC(O)-[4- | H₂ | H |
| | (CH₂)₂O(CH₂)₂OH-piperazin-1-yl]}CH₂— | | |
| 167 | —CH₂CH[CH₂OC(O)-(4-C(O)O-benzyl- | H₂ | H |
| | piperazin-1-yl)]CH₂— | | |
| 168 | —CH₂CH[CH₂OC(O)-(3R)-3-OH- | H₂ | H |
| | pyrrolidin-1-yl]CH₂— | | |
| 169 | —CH₂CH[CH₂OC(O)NH-(2-OCH₃- | H₂ | H |
| | benzyl)]CH₂— | | |
| 170 | —CH₂CH[CH₂OC(O)-(4-CH₂- | H₂ | H |
| | benzo[1,3]dioxol-5-yl-piperazin-1- | | |
| | yl)]CH₂— | | |
| 171 | —CH₂CH{CH₂OC(O)-[(3S)-3-N(CH₃)₂- | H₂ | H |
| | pyrrolidin-1-yl]}CH₂— | | |
| 172 | —CH₂CH{CH₂OC(O)-[4-NHC(O)—OC(CH₃)₃- | H₂ | H |
| | piperidin-1-yl]}CH₂— | | |
| 173 | —CH₂{C(OH)[CH₂OC(O)NH—(CH₂)₂N(CH₃)₂]}CH₂— | H₂ | H |
| 174 | —CH₂{C(OH)[CH₂OC(O)NH-(2-OCH₃- | H₂ | H |
| | benzyl)]}CH₂— | | |
| 175 | —CH₂(C(OH){CH₂OC(O)-[4-NH—C(O)OC(CH₃)₃- | H₂ | H |
| | piperidin-1-yl]})CH₂— | | |
| 176 | —CH₂{C(OH)[CH₂OC(O)-(4-NH₂- | H₂ | H |
| | piperidin-1-yl)]}CH₂— | | |
| 177 | —CH₂(C(OH){CH₂OC(O)NH-[1- | H₂ | H |
| | C(O)OC(CH₃)₃-piperidin-4-yl]})CH₂— | | |
| 178 | —CH₂{C(OH)[CH₂OC(O)NH-piperidin-4- | H₂ | H |
| | yl]}CH₂— | | |
| 179 | —(CH₂)₂CH[OC(O)NH—(CH₂)₂OCH₃](CH₂)₂— | H₂ | H |
| 180 | —(CH₂)₂CH[OC(O)NH—CH(CH₃)₂](CH₂)₂— | H₂ | H |
| 181 | —(CH₂)₂CH{OC(O)-[4-(4-OH-phenyl)- | H₂ | H |
| | piperazin-1-yl]}(CH₂)₂— | | |
| 182 | —(CH₂)₂CH[OC(O)NH—CH(S—CH₃)—CH₂OCH₃](CH₂)₂— | H₂ | H |
| 183 | —(CH₂)₂CH{OC(O)NH(CH₂)₂-[3,4- | H₂ | H |
| | (OCH₃)₂-phenyl]}(CH₂)₂— | | |
| 184 | —(CH₂)₂CH[OC(O)-(4-pyridin-4-yl- | H₂ | H |
| | piperazin-1-yl)](CH₂)₂— | | |
| 185 | —(CH₂)₂CH[OC(O)-morpholin-4- | H₂ | H |
| | yl](CH₂)₂— | | |
| 186 | —(CH₂)₂CH[OC(O)NH—(CH₂)₃N(CH₃)₂](CH₂)₂— | H₂ | H |
| 187 | —(CH₂)₂CH[OC(O)NH-(1-benzyl- | H₂ | H |
| | piperidin-4-yl)](CH₂)₂— | | |
| 188 | —CH₂CH(OH)—CH(OH)CH₂— | H₂ | 3-Br |
| 189 | —CH₂CH(OH)—CH(OH)CH₂— | H₂ | 3-OH |
| 190 | —CH₂CH(OH)—CH(OH)CH₂— | H₂ | 3-CH₂OH |
| 191 | —CH₂CH(OH)—CH(OH)CH₂— | H₂ | 3-NO₂ |
| 192 | —CH₂CH(OH)—CH(OH)CH₂— | H₂ | 3-NH₂ |
| 193 | —CH₂CH(OH)—CH(OH)CH₂— | H₂ | 3-[CH₂-(4-CH₃-piperazin- |
| | | | 1-yl)] |
| 194 | —CH₂CH(OH)—CH(OH)CH₂— | H₂ | 3-CH₂-morpholin-4-yl |
| 195 | —CH₂CH(OH)—CH(OH)CH₂— | H₂ | 3-[CH₂NH-(1-CH₃- |
| | | | piperidin-4-yl)] |
| 196 | —CH₂CH(OH)—CH(OH)CH₂— | H₂ | 3-[CH₂N(CH₃)-(1-CH₃- |
| | | | piperidin-4-yl)] |
| 197 | —CH₂CH(OH)—CH(OH)CH₂— | H₂ | 3-[CH₂NH(CH₂)₃-(4-CH₃- |
| | | | piperazin-1-yl)] |
| 198 | —CH₂CH(OH)—CH(OH)CH₂— | H₂ | 3-[CH₂NH(CH₂)₂OCH₃] |
| 199 | —CH₂CH(OH)—CH(OH)CH₂— | H₂ | 3-[CH₂S(CH₂)₂—N(CH₃)₂] |
| 200 | —CH₂CH(OH)—CH(OH)CH₂— | H₂ | 3-[CH═N—NH-4,5-dihydro- |
| | | | 1H-imidazol-2-yl] |
| 201 | —CH₂CH(OH)—CH(OH)CH₂— | H₂ | 3-[CH═CHCH₂-1H- |
| | | | imidazol-1-yl] |
| 202 | —CH₂CH(OH)—CH(OH)CH₂— | H₂ | 3-[CH₂-1H-imidazol-1-yl] |
| 203 | —CH₂CH(OH)—CH(OH)CH₂— | H₂ | 3-[O(CH₂)₂-morpholin-4- |
| | | | yl] |
| 204 | —CH₂CH(OH)—CH(OH)CH₂— | H₂ | 3-[NH-(1-CH₃-piperidin-4- |
| | | | yl)] |
| 205 | —CH₂CH(OH)—CH(OH)CH₂— | H₂ | 3-[NHC(O)CH₂-(4-CH₃- |
| | | | piperazin-1-yl)] |
| 206 | —CH₂CH(OH)—CH(OH)CH₂— | H₂ | 3-[NH-4,5-dihydro-1H- |
| | | | imidazol-2-yl] |
| 207 | —CH₂CH(CH₂OH)CH₂— | H₂ | 3-Br-9-C(O)H |
| 208 | —CH₂CH(CH₂OH)CH₂— | H₂ | 3-Br-9-(CH₂-morpholin-4- |
| | | | yl) |
| 209 | —CH₂CH(CH₂OH)CH₂— | H₂ | 3-Br-9-CH₂OCH(CH₃)₂ |
| 210 | —CH₂CH(CH₂OH)CH₂— | H₂ | 3,9-[CH₂OCH(CH₃)₂]₂ |
| 211 | —CH₂CH(CH₂OH)CH₂— | H₂ | 3-CH₂OCH(CH₃)₂ |
| 212 | —CH₂CH(CH₂OH)CH₂— | H₂ | 9-CH₂OH |
| 213 | —CH₂CH(CH₂OH)CH₂— | H₂ | 3-CH₂NHCH(CH₃)₂ |
| 214 | —CH₂CH(CH₂OH)CH₂— | H₂ | 3,9-(CH₂-morpholin-4-yl)₂ |
| 215 | —CH₂CH(CH₂OH)CH₂— | H₂ | 3,9-(CH₂OH)₂ |
| 216 | —CH₂CH(CH₂OH)CH₂— | H₂ | 3-(CH═CH-pyridin-2-yl) |
| 217 | —CH₂CH(CH₂OH)CH₂— | H₂ | 3-[CH═CH-(4-CH₃- |
| | | | thiazol-5-yl)] |
| 218 | —CH₂CH(CH₂OH)CH₂— | H₂ | 3-[CH═CH—C(O)OH] |
| 219 | —CH₂CH(CH₂OH)CH₂— | H₂ | 3-(CH═CHCH₂-1H- |
| | | | imidazol-1-yl |
| 220 | —CH₂CH(CH₂OH)CH₂— | H₂ | 3-(CH═CH-1H-imidazol- |
| | | | 1-yl) |
| 221 | —CH₂CH(CH₂OH)CH₂— | H₂ | 3-(CH═CHCH₂NH-(4,5- |
| | | | dihydro-1H-imidazol-2- |
| | | | yl)] |
| 222 | —CH₂CH(CH₂OH)CH₂— | H₂ | 3-[CH═CHC(O)—NH(CH₂)₂N(CH₃)₂] |
| 223 | —CH₂CH(CH₂OH)CH₂— | H₂ | 3-(CH═CHCH₂-1H- |
| | | | imidazol-1-yl)-9-CH₂OH |

An example of the present invention is a compound of formula (I) or a form thereof represented by a compound selected from:

| | |
|---|---|
| Cpd. No. 1 | 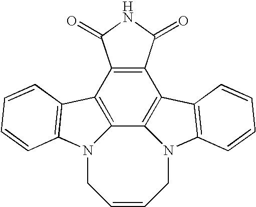 |
| Cpd. No. 2 | 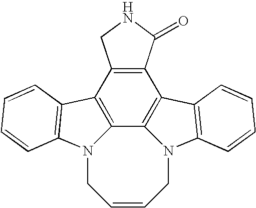 |
| Cpd. No. 3 | 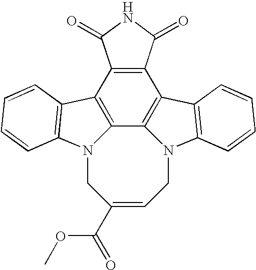 |
| Cpd. No. 4 | 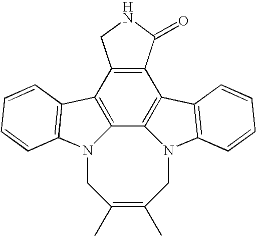 |
| Cpd. No. 5 | 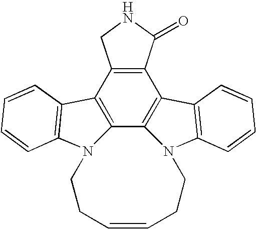 |
| Cpd. No. 6 | 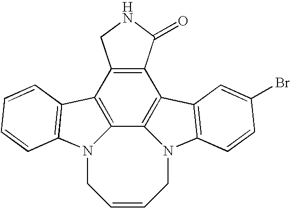 |
| Cpd. No. 7 | 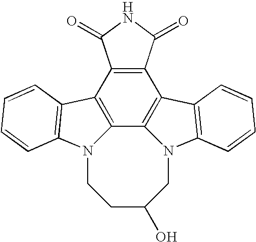 |
| Cpd. No. 8 | 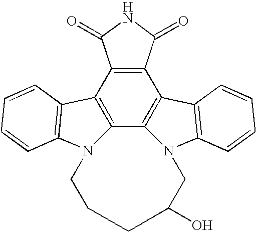 |
| Cpd. No. 9 | 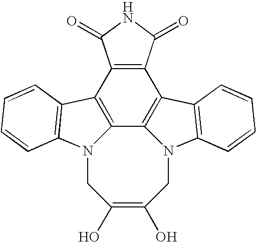 |
| Cpd. No. 10 | 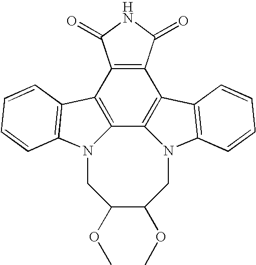 |
| Cpd. No. 11 | 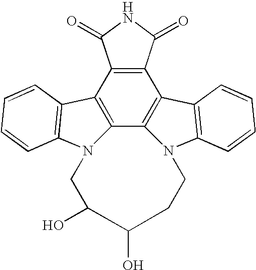 |
| Cpd. No. 12 | 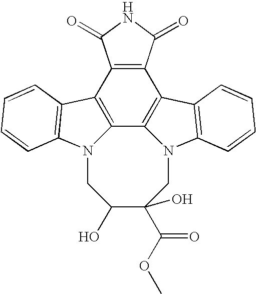 |
| Cpd. No. 13 | 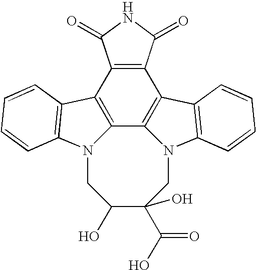 |
| Cpd. No. 14 | 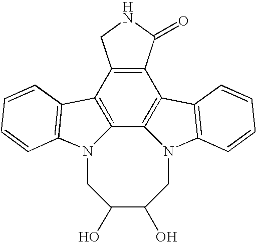 |
| Cpd. No. 15 | 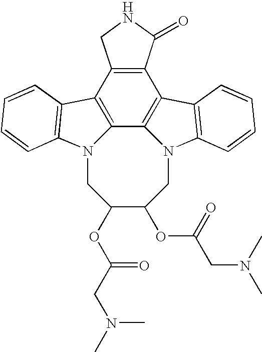 |
| Cpd. No. 16 | 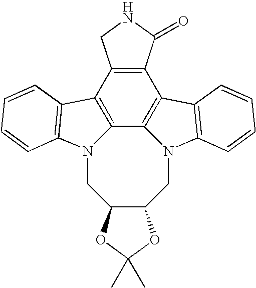 |
| Cpd. No. 17 | 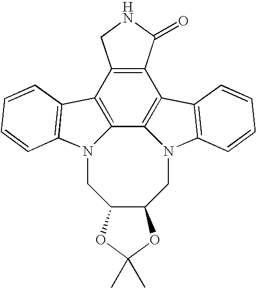 |
| Cpd. No. 18 | 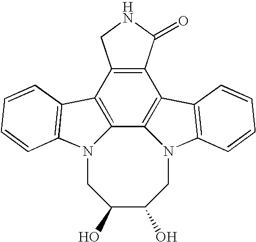 |
| Cpd. No. 19 | 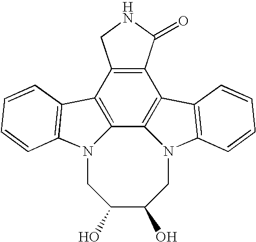 |
| Cpd. No. 20 | 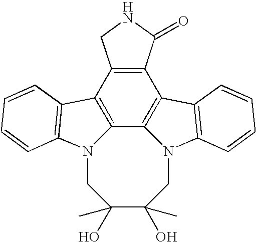 |
| Cpd. No. 21 | 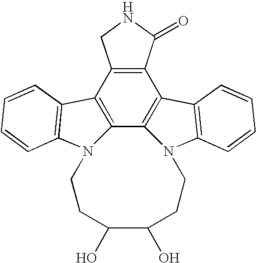 |
| Cpd. No. 22 | 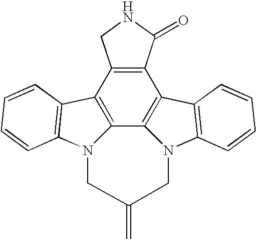 |
| Cpd. No. 23 | 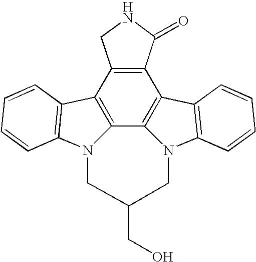 |
| Cpd. No. 24 | 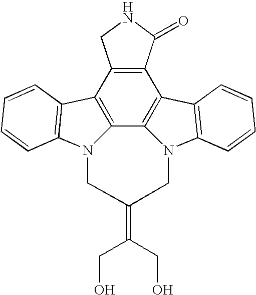 |
| Cpd. No. 25 | 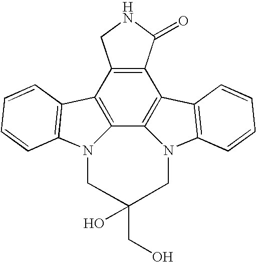 |
| Cpd. No. 26 | 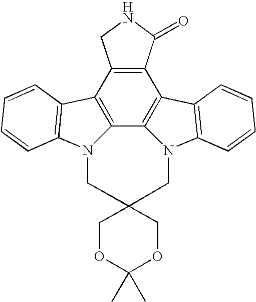 |
| Cpd. No. 27 | 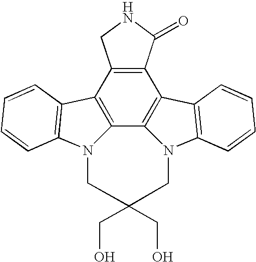 |
| Cpd. No. 28 | 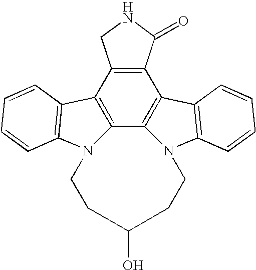 |
| Cpd. No. 29 | 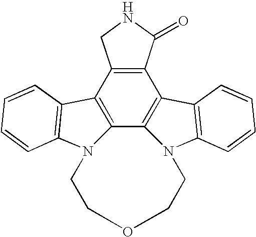 |
| Cpd. No. 30 | 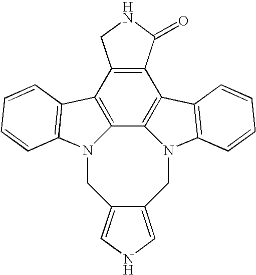 |
| Cpd. No. 31 | 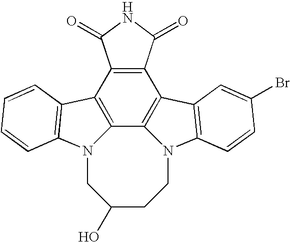 |
| Cpd. No. 32 | 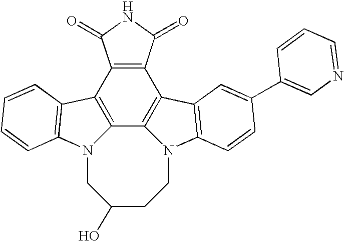 |
| Cpd. No. 33 | 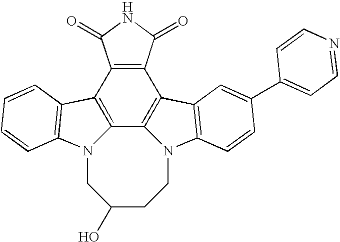 |
| Cpd. No. 34 | 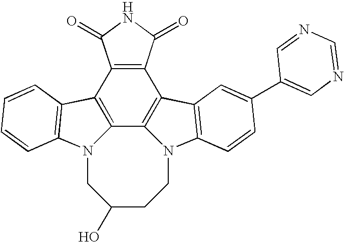 |
| Cpd. No. 35 | 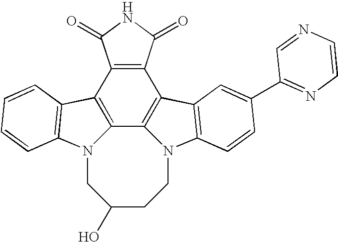 |
| Cpd. No. 36 | 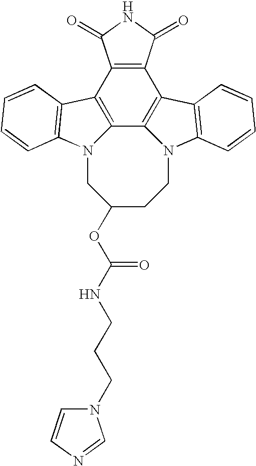 |
| Cpd. No. 37 | 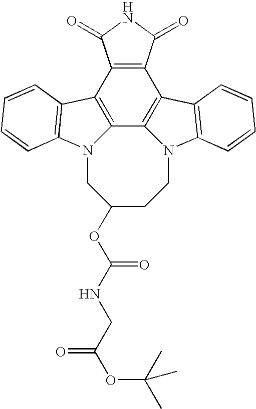 |
| Cpd. No. 38 | 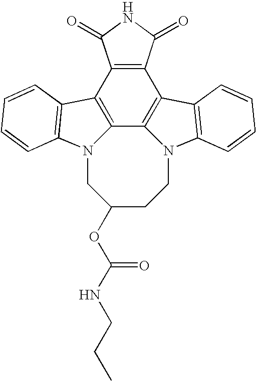 |
| Cpd. No. 39 | 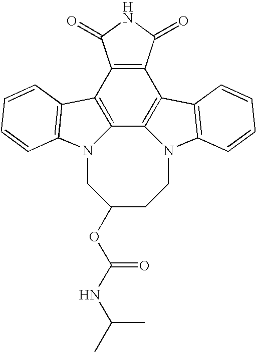 |
| Cpd. No. 40 | 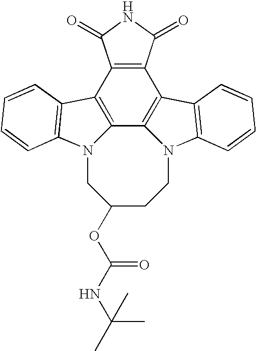 |
| Cpd. No. 41 | 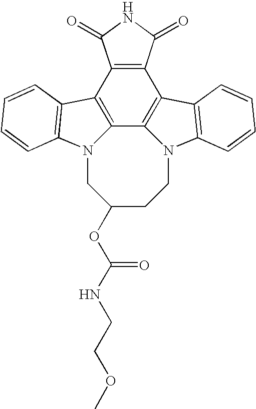 |
| Cpd. No. 42 | 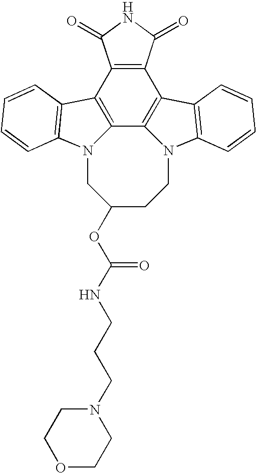 |
| Cpd. No. 43 | 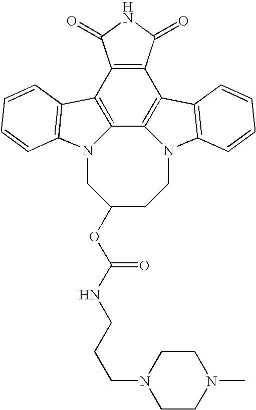 |
| Cpd. No. 44 | 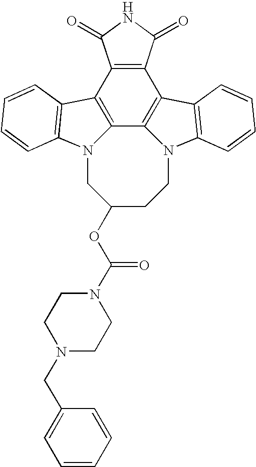 |
| Cpd. No. 45 | 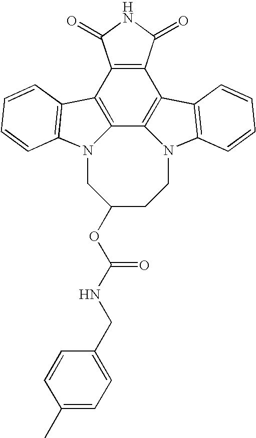 |
| Cpd. No. 46 | 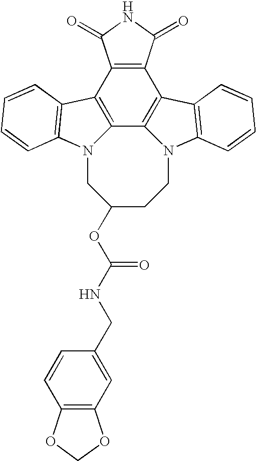 |
| Cpd. No. 47 | 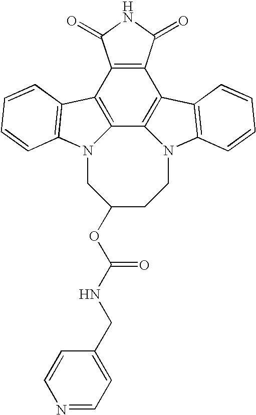 |
| Cpd. No. 48 | 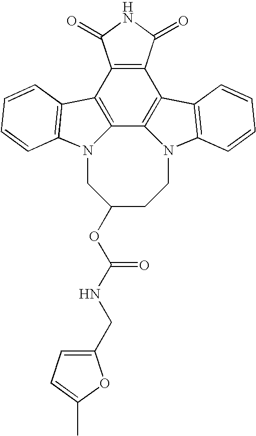 |
| Cpd. No. 49 | 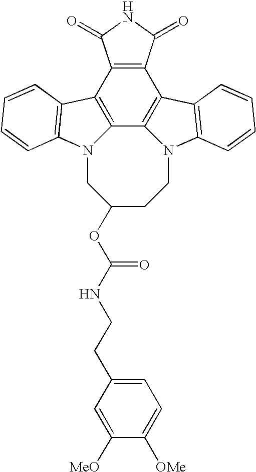 |
| Cpd. No. 50 | 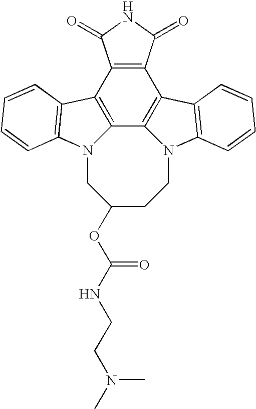 |
| Cpd. No. 51 | 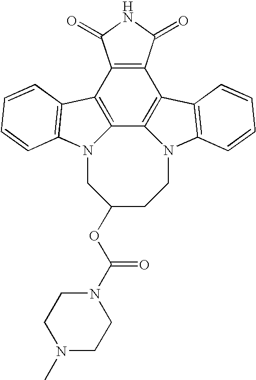 |
| Cpd. No. 52 | 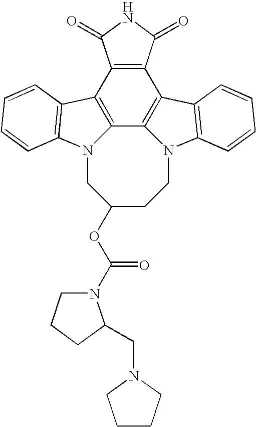 |
| Cpd. No. 53 | 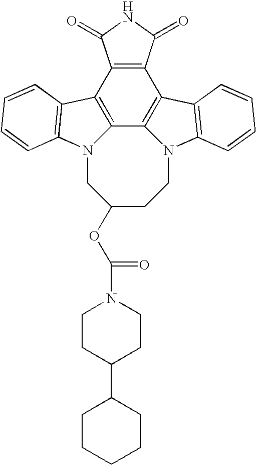 |
| Cpd. No. 54 | 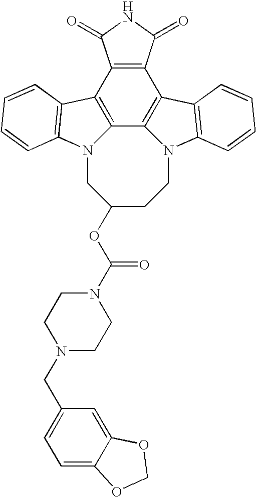 |
| Cpd. No. 55 | 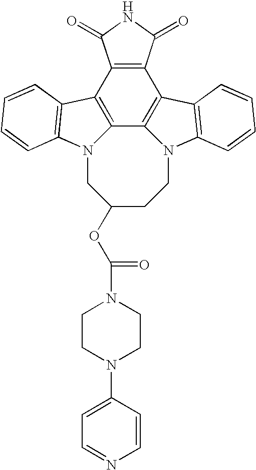 |
| Cpd. No. 56 | 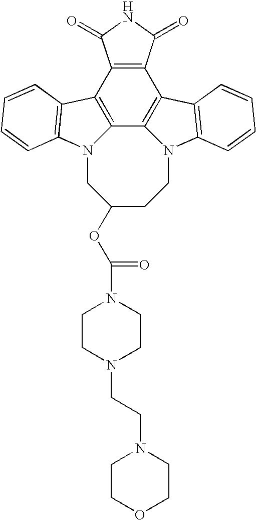 |
| Cpd. No. 57 | 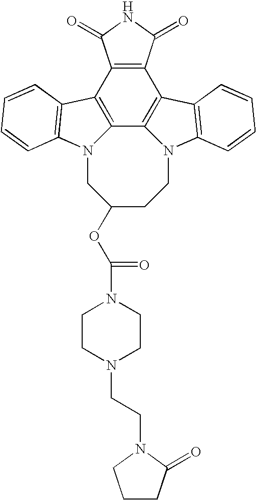 |
| Cpd. No. 58 | 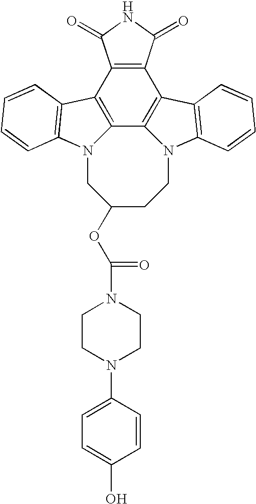 |
| Cpd. No. 59 | 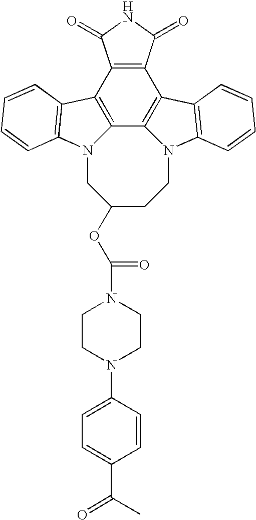 |
| Cpd. No. 60 | 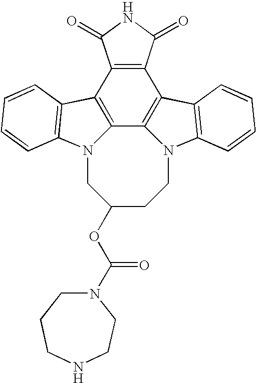 |
| Cpd. No. 61 | 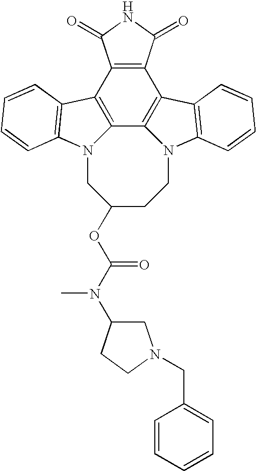 |
| Cpd. No. 62 | 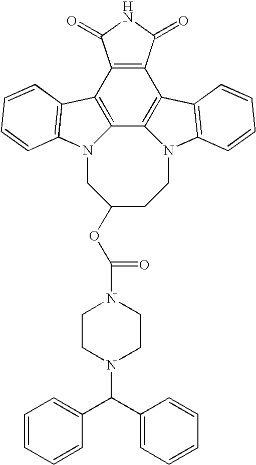 |
| Cpd. No. 63 | 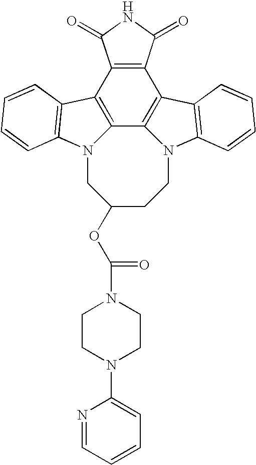 |
| Cpd. No. 64 | 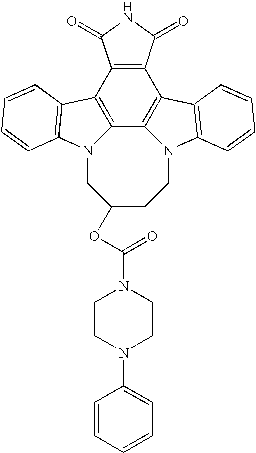 |
| Cpd. No. 65 | 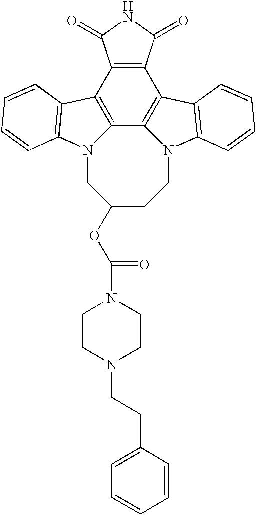 |
| Cpd. No. 66 | 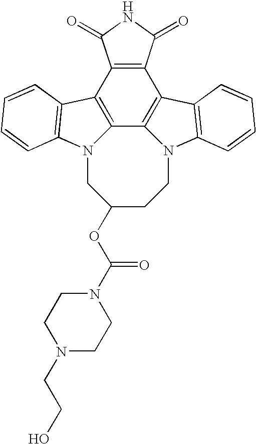 |
| Cpd. No. 67 | 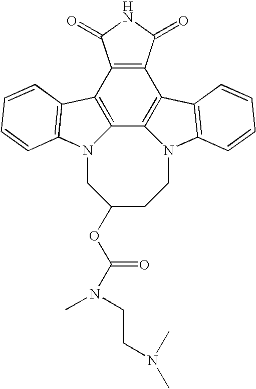 |
| Cpd. No. 68 | 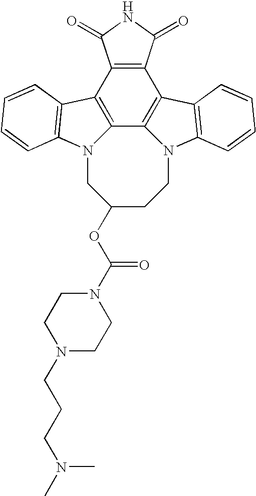 |
| Cpd. No. 69 | 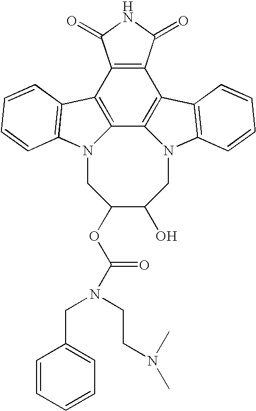 |
| Cpd. No. 70 | 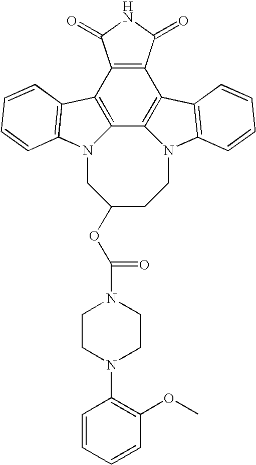 |
| Cpd. No. 71 | 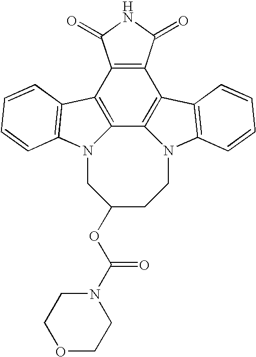 |
| Cpd. No. 72 | 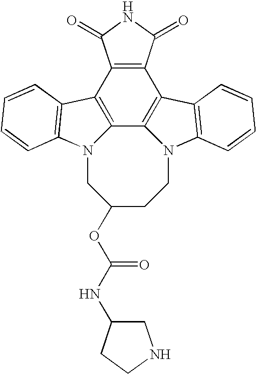 |
| Cpd. No. 73 | 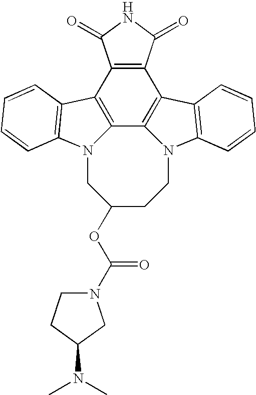 |
| Cpd. No. 74 | 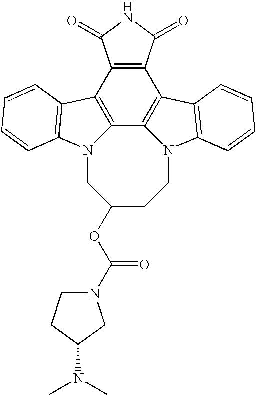 |
| Cpd. No. 75 | 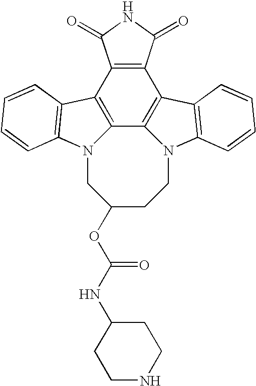 |
| Cpd. No. 76 | 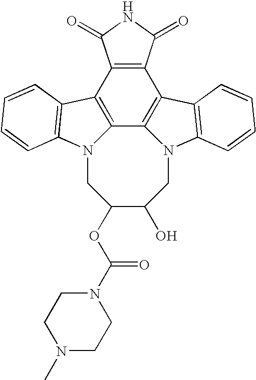 |
| Cpd. No. 77 | 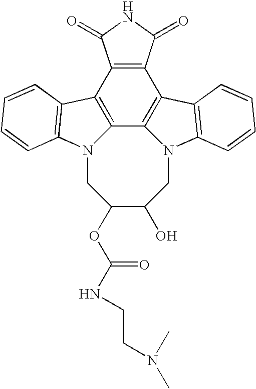 |
| Cpd. No. 78 | 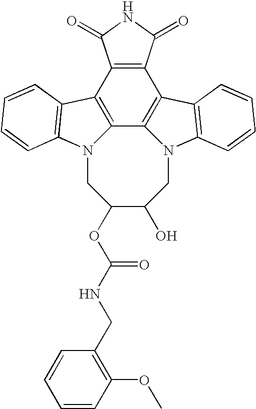 |
| Cpd. No. 79 | 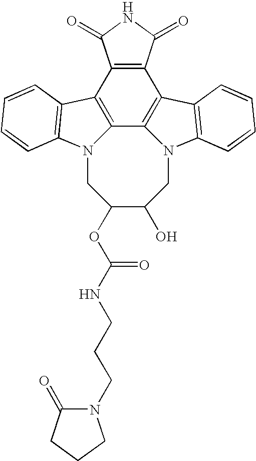 |
| Cpd. No. 80 | 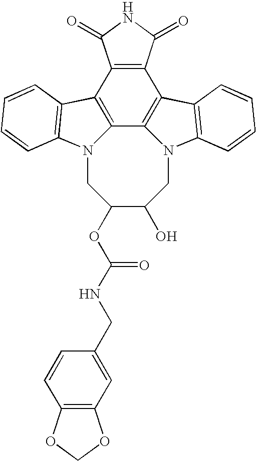 |
| Cpd. No. 81 | 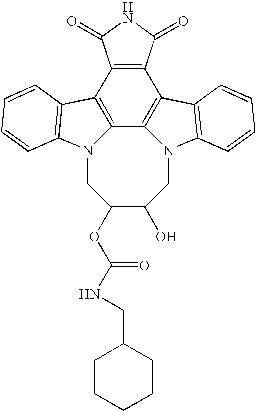 |
| Cpd. No. 82 | 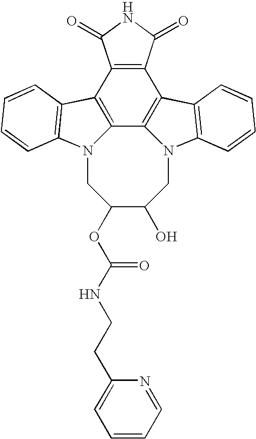 |
| Cpd. No. 83 | 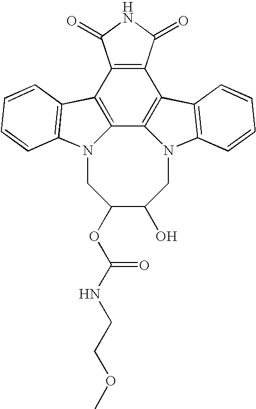 |
| Cpd. No. 84 | 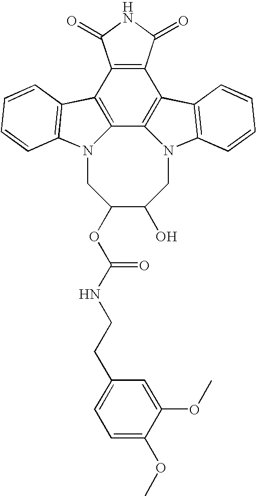 |
| Cpd. No. 85 | 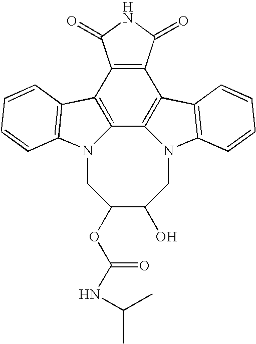 |
| Cpd. No. 86 | 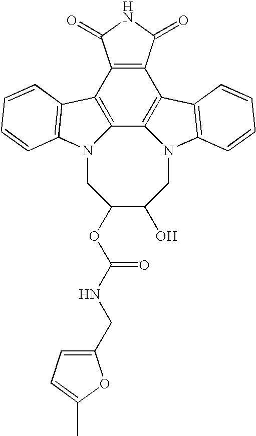 |
| Cpd. No. 87 | 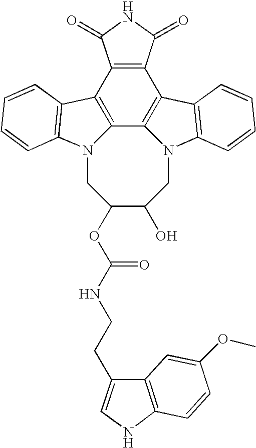 |
| Cpd. No. 88 | 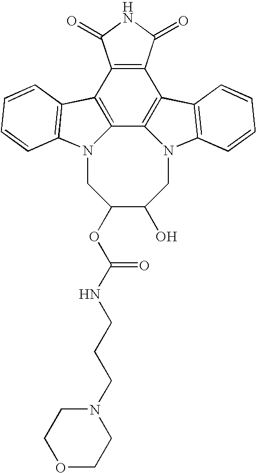 |
| Cpd. No. 89 | 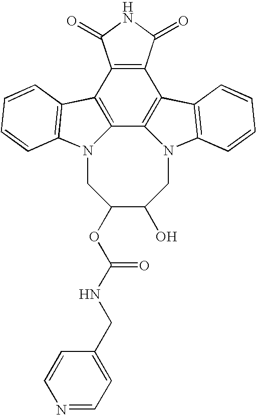 |
| Cpd. No. 90 | 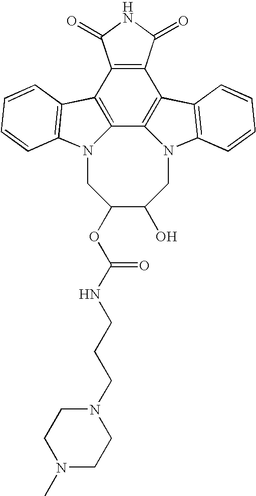 |
| Cpd. No. 91 | 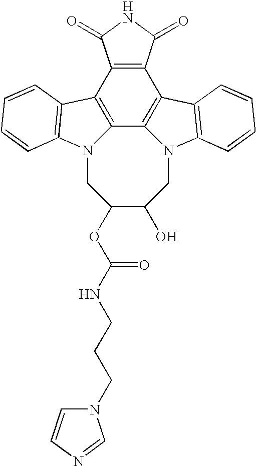 |
| Cpd. No. 92 | 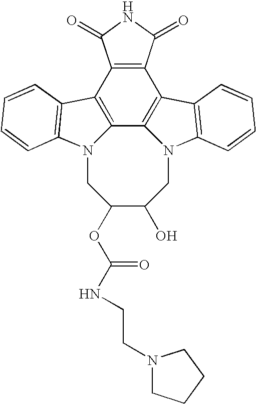 |
| Cpd. No. 93 | 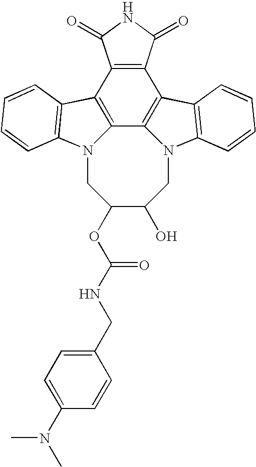 |
| Cpd. No. 94 | 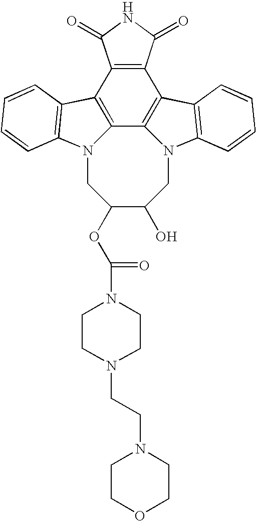 |
| Cpd. No. 95 | 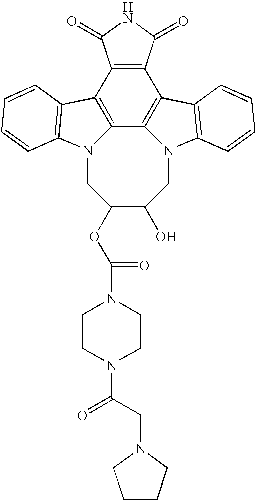 |
| Cpd. No. 96 | 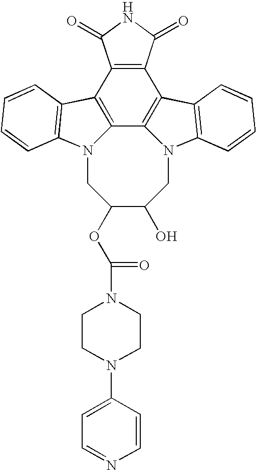 |
| Cpd. No. 97 | 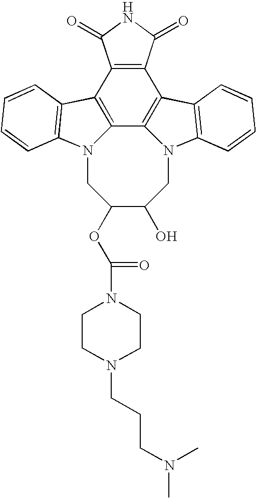 |
| Cpd. No. 98 | 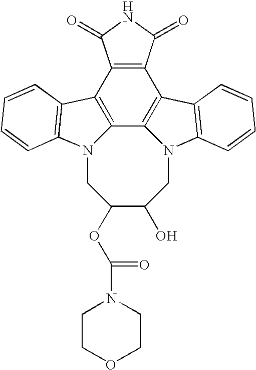 |
| Cpd. No. 99 | 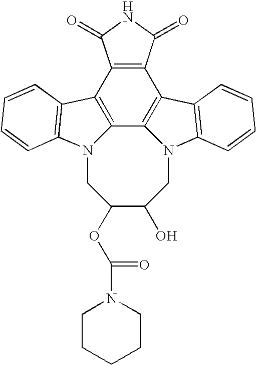 |
| Cpd. No. 100 | 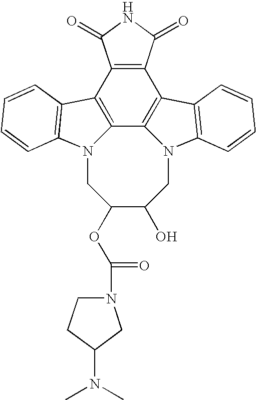 |
| Cpd. No. 101 | 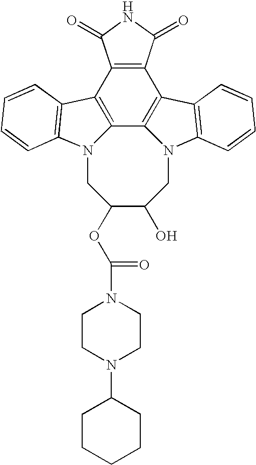 |
| Cpd. No. 102 | 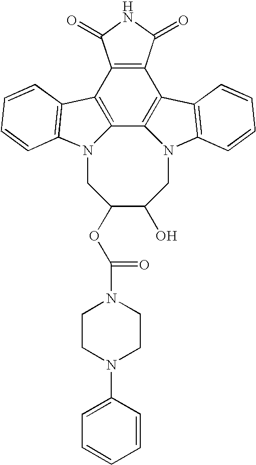 |
| Cpd. No. 103 | 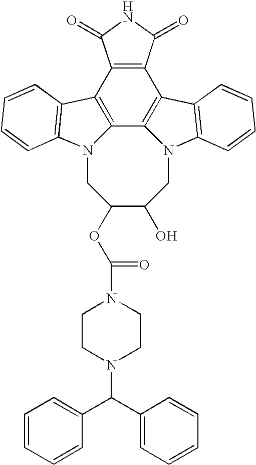 |
| Cpd. No. 104 | 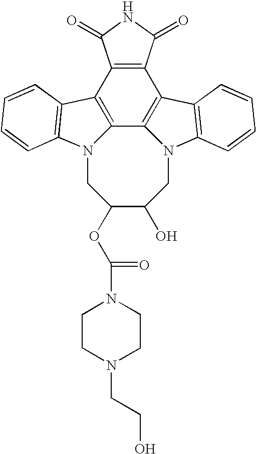 |
| Cpd. No. 105 | 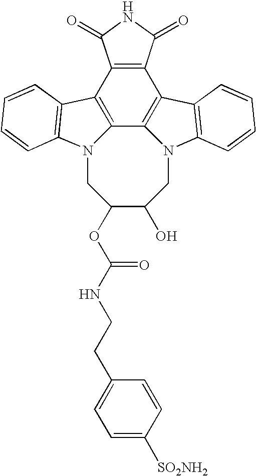 |
| Cpd. No. 106 | 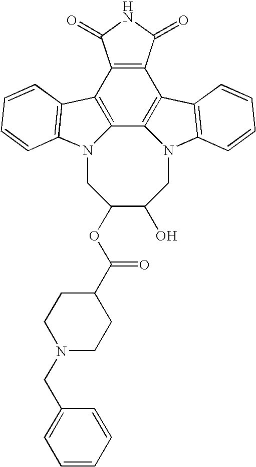 |
| Cpd. No. 107 | 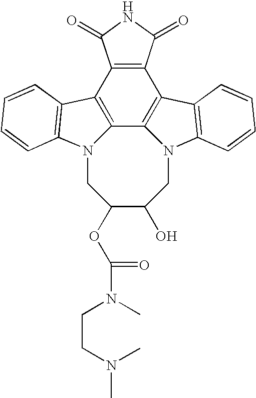 |
| Cpd. No. 108 | 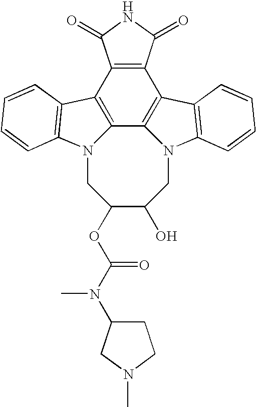 |
| Cpd. No. 109 | 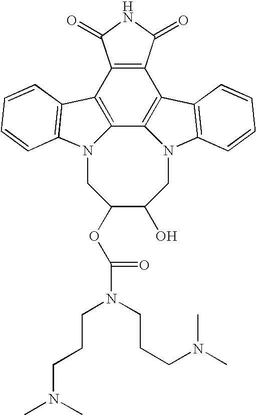 |
| Cpd. No. 110 | 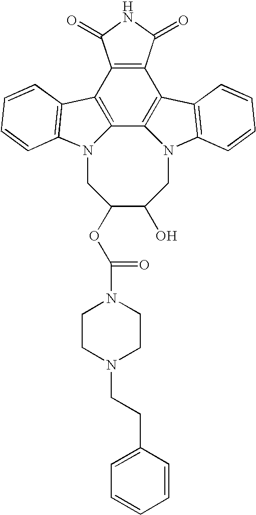 |
| Cpd. No. 111 | 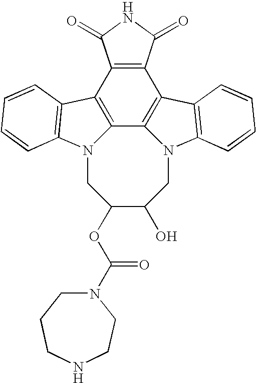 |
| Cpd. No. 112 | 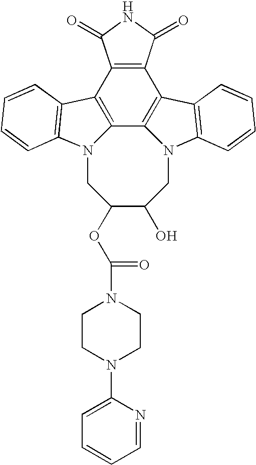 |
| Cpd. No. 113 | 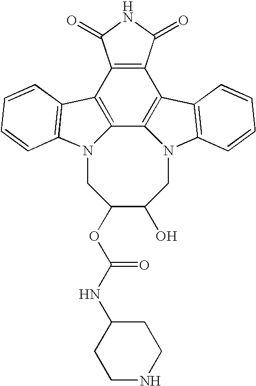 |
| Cpd. No. 114 | 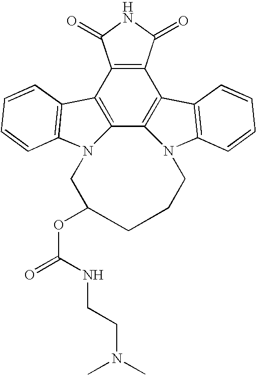 |
| Cpd. No. 115 | 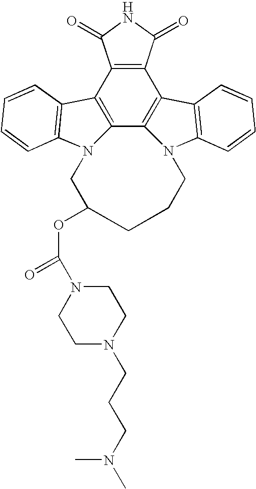 |
| Cpd. No. 116 | 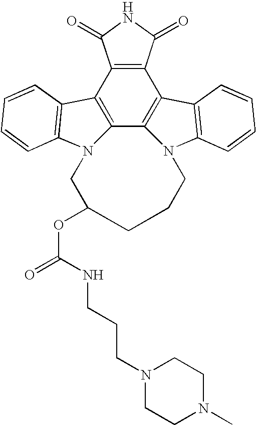 |
| Cpd. No. 117 | 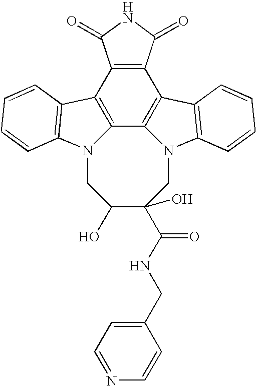 |
| Cpd. No. 118 | 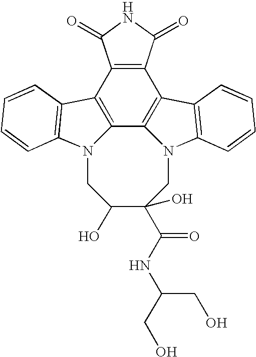 |
| Cpd. No. 119 | 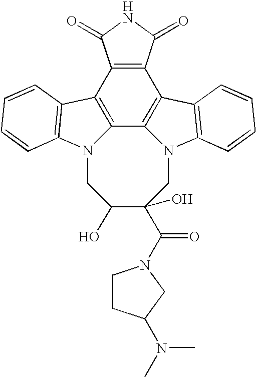 |
| Cpd. No. 120 | 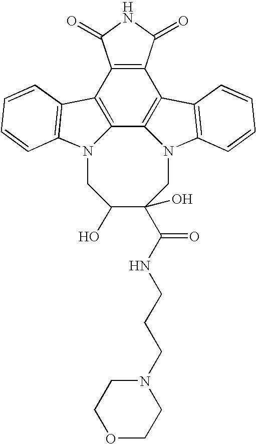 |
| Cpd. No. 121 | 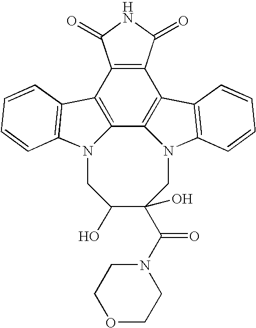 |
| Cpd. No. 122 | 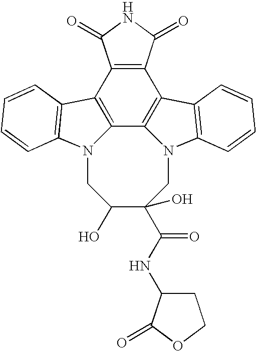 |
| Cpd. No. 123 | 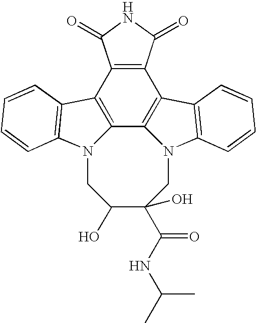 |
| Cpd. No. 124 | 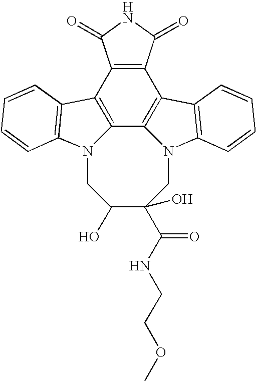 |
| Cpd. No. 125 | 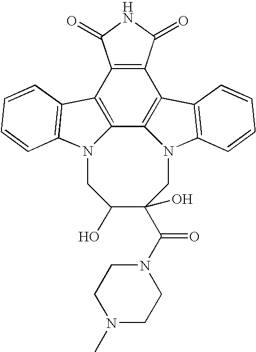 |
| Cpd. No. 126 | 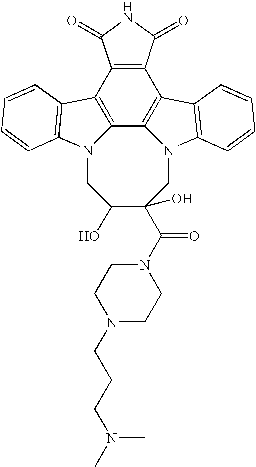 |
| Cpd. No. 127 | 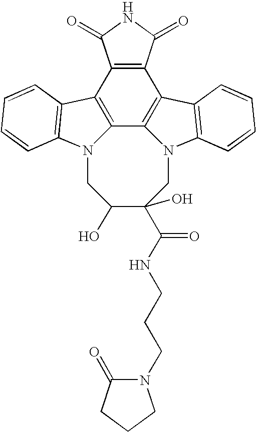 |
| Cpd. No. 128 | 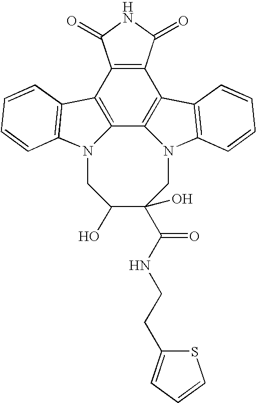 |
| Cpd. No. 129 | 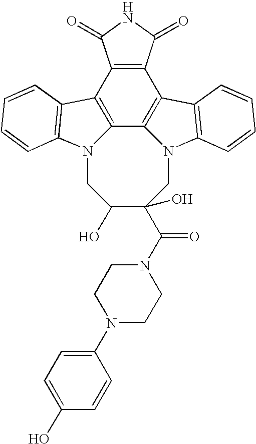 |
| Cpd. No. 130 | 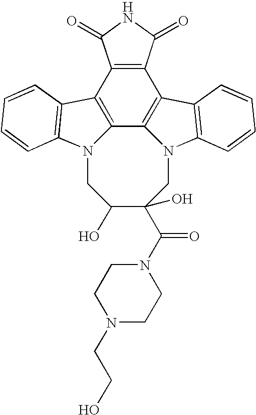 |
| Cpd. No. 131 | 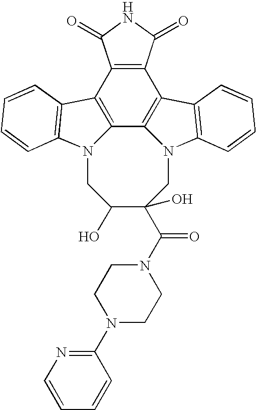 |
| Cpd. No. 132 | 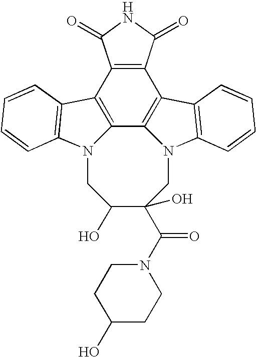 |
| Cpd. No. 133 | 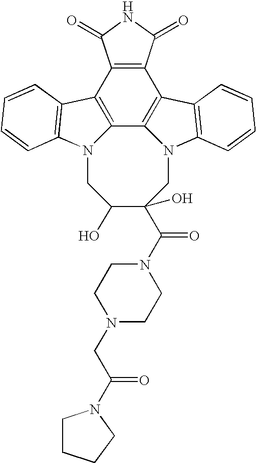 |
| Cpd. No. 134 | 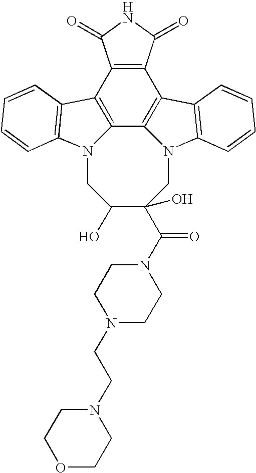 |
| Cpd. No. 135 | 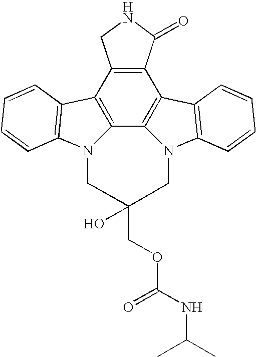 |
| Cpd. No. 136 | 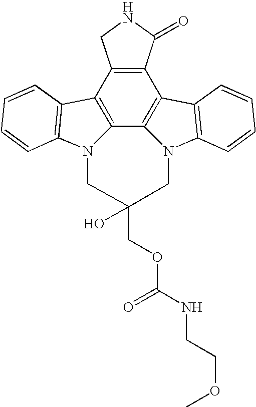 |
| Cpd. No. 137 | 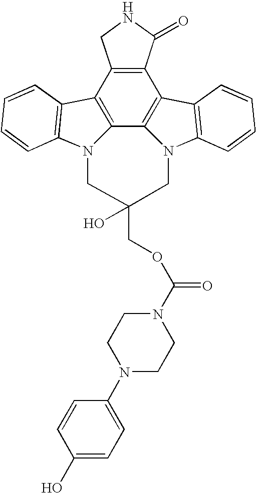 |
| Cpd. No. 138 | 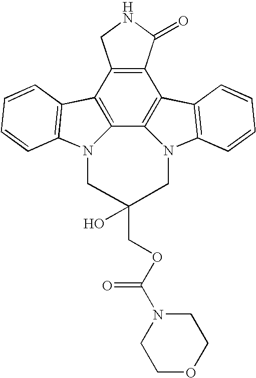 |
| Cpd. No. 139 | 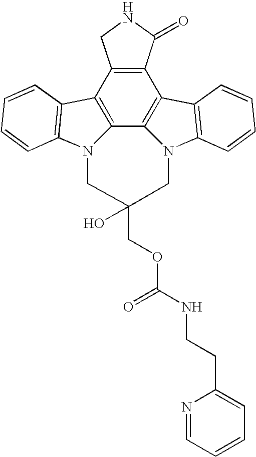 |
| Cpd. No. 140 | 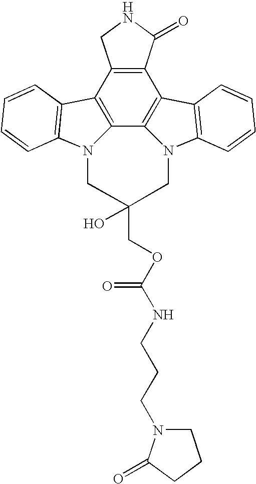 |
| Cpd. No. 141 | 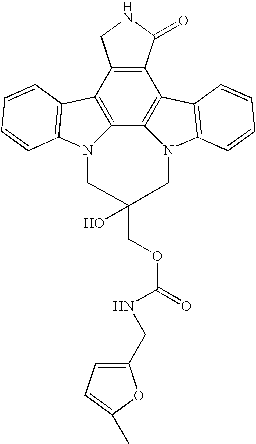 |
| Cpd. No. 142 | 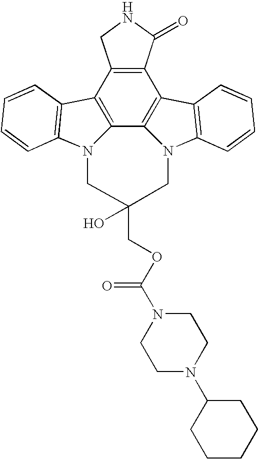 |
| Cpd. No. 143 | 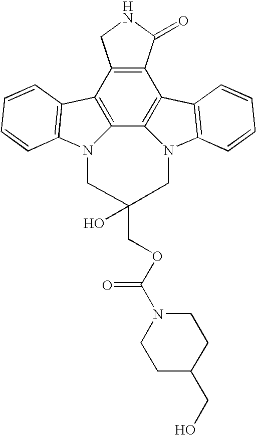 |
| Cpd. No. 144 | 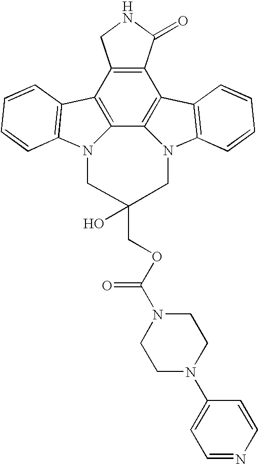 |
| Cpd. No. 145 | 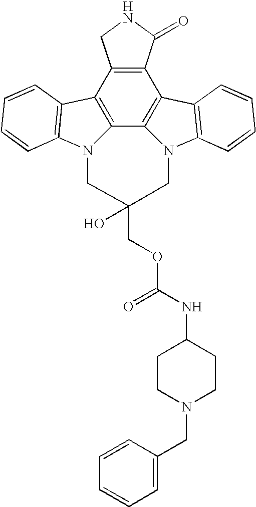 |
| Cpd. No. 146 | 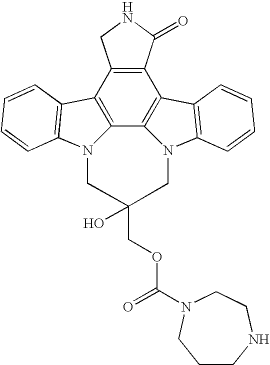 |
| Cpd. No. 147 | 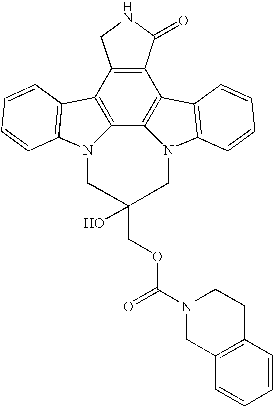 |
| Cpd. No. 148 | 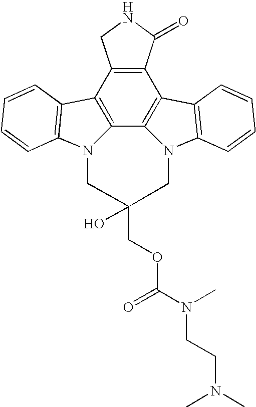 |
| Cpd. No. 149 | 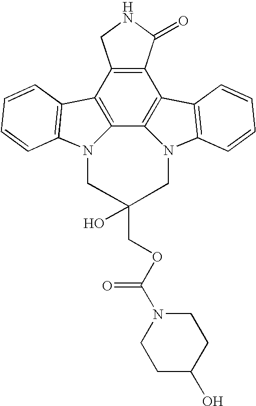 |
| Cpd. No. 150 | 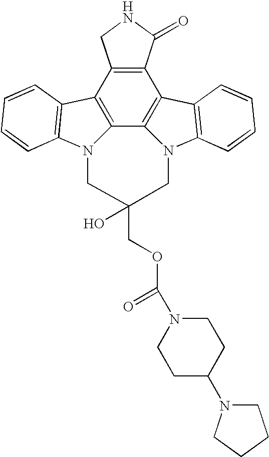 |
| Cpd. No. 151 | 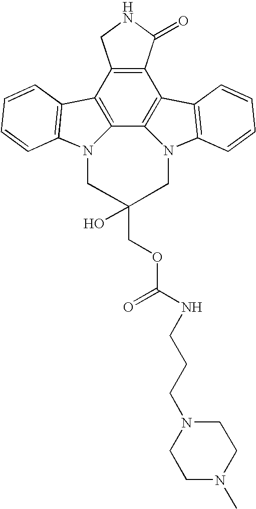 |
| Cpd. No. 152 | 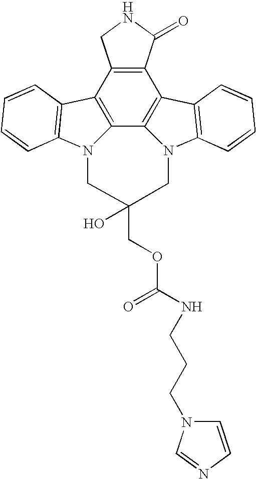 |
| Cpd. No. 153 | 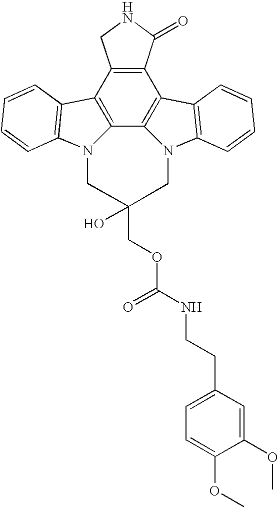 |
| Cpd. No. 154 | 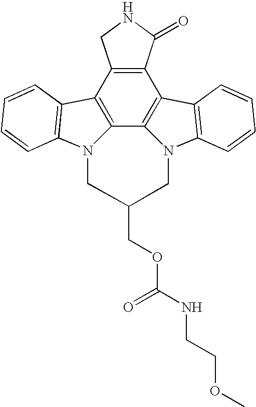 |
| Cpd. No. 155 | 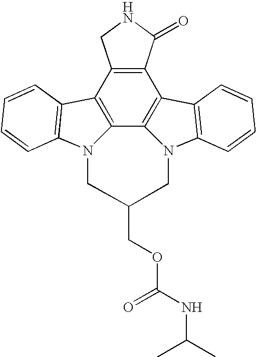 |
| Cpd. No. 156 | 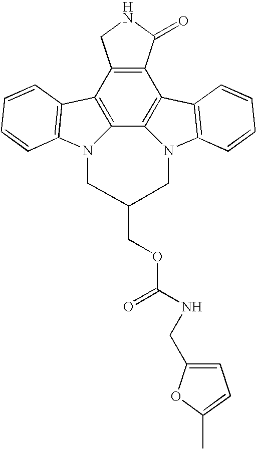 |
| Cpd. No. 157 | 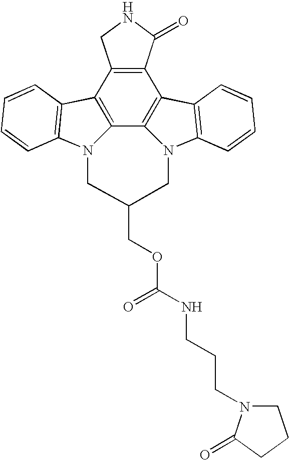 |
| Cpd. No. 158 | 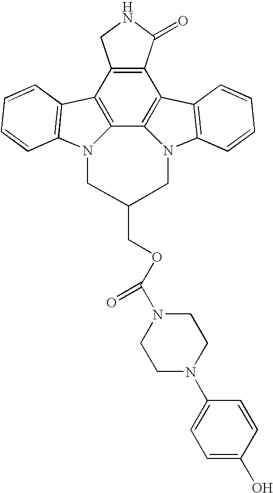 |
| Cpd. No. 159 | 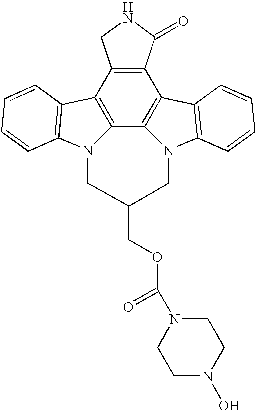 |
| Cpd. No. 160 | 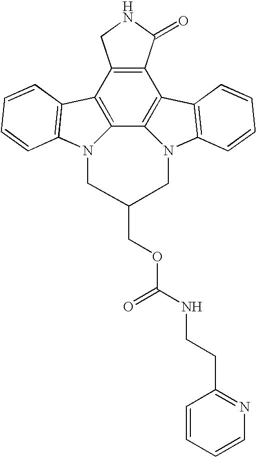 |
| Cpd. No. 161 | 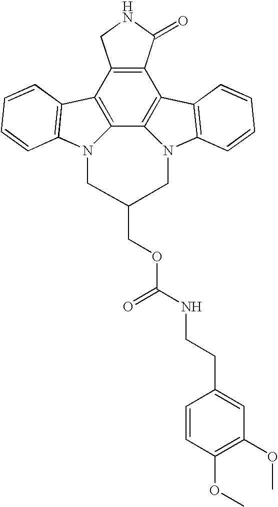 |
| Cpd. No. 162 | 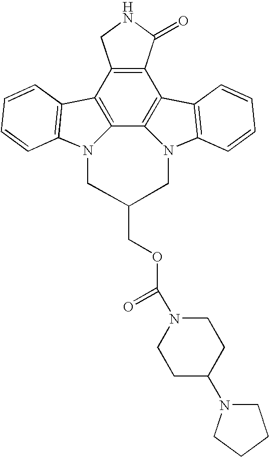 |
| Cpd. No. 163 | 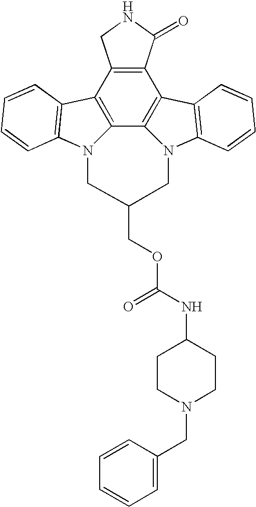 |
| Cpd. No. 164 | 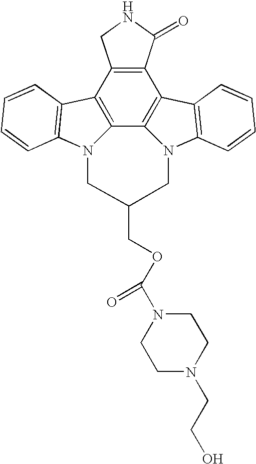 |
| Cpd. No. 165 | 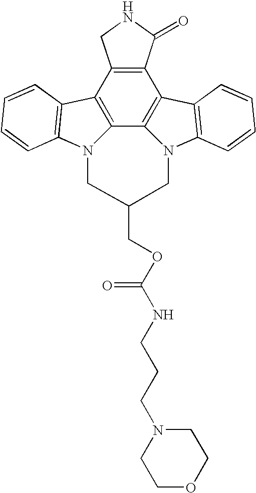 |
| Cpd. No. 166 | 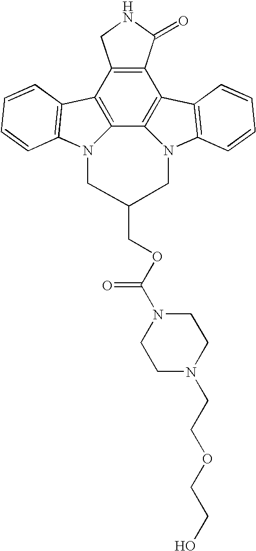 |
| Cpd. No. 167 | 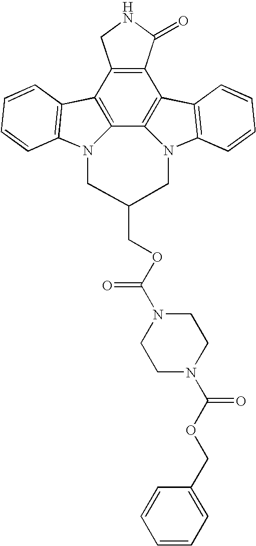 |
| Cpd. No. 168 | 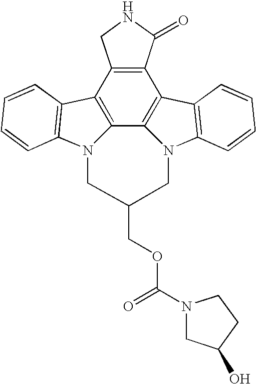 |
| Cpd. No. 169 | 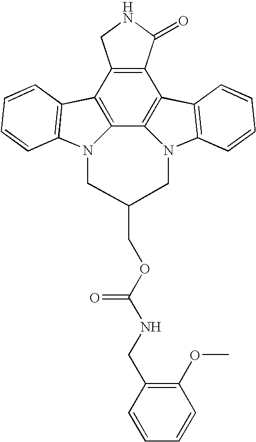 |
| Cpd. No. 170 | 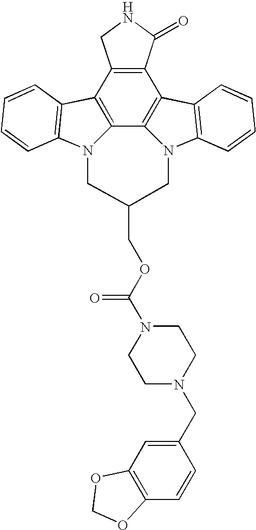 |
| Cpd. No. 171 | 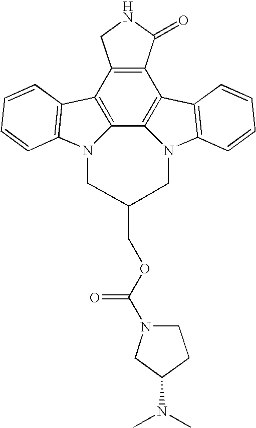 |
| Cpd. No. 172 | 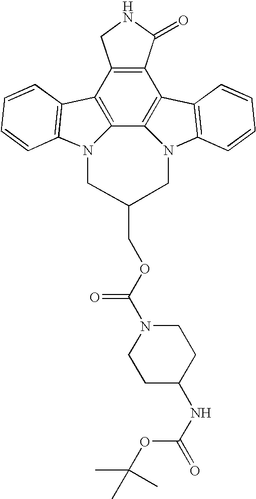 |
| Cpd. No. 173 | 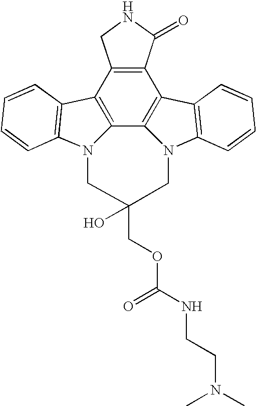 |
| Cpd. No. 174 | 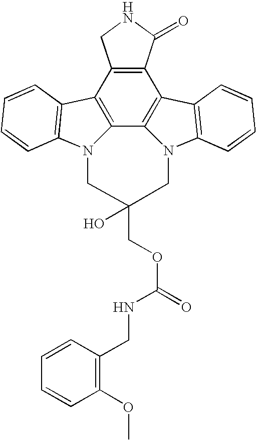 |
| Cpd. No. 175 | 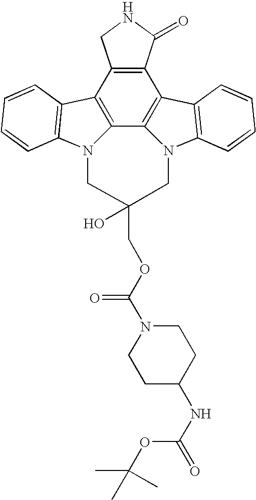 |
| Cpd. No. 176 | 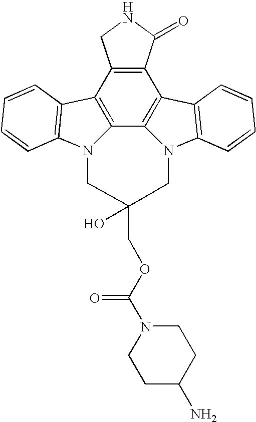 |
| Cpd. No. 177 | 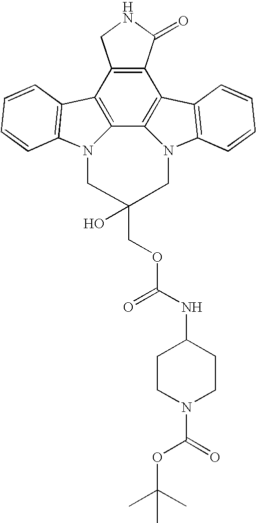 |
| Cpd. No. 178 | 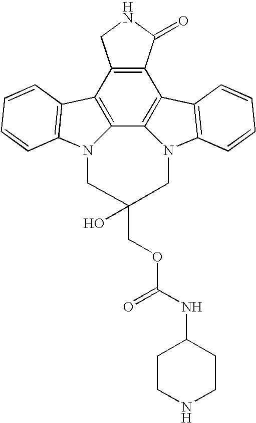 |
| Cpd. No. 179 | 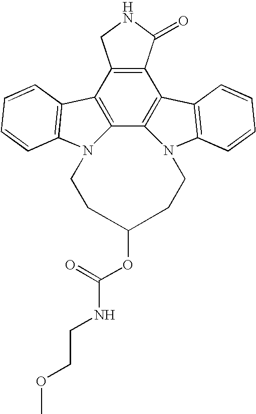 |
| Cpd. No. 180 | 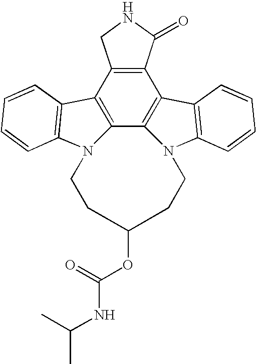 |
| Cpd. No. 181 | 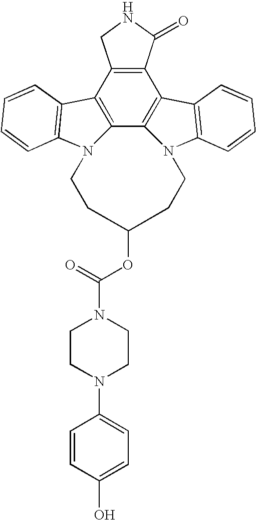 |
| Cpd. No. 182 | 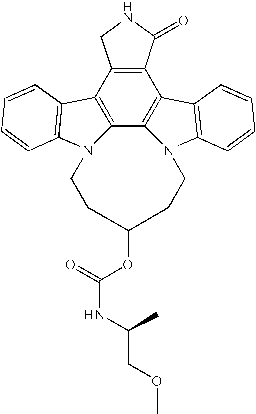 |
| Cpd. No. 183 | 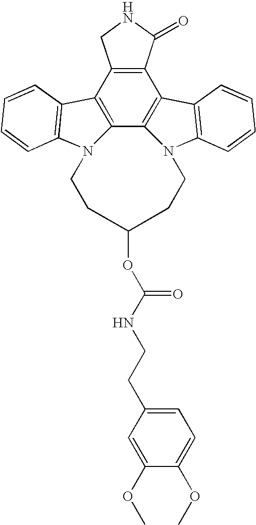 |
| Cpd. No. 184 | 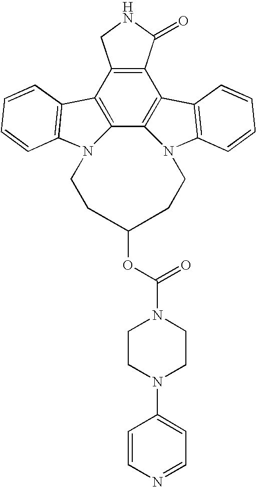 |
| Cpd. No. 185 | 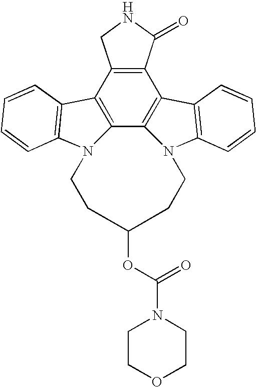 |
| Cpd. No. 186 | 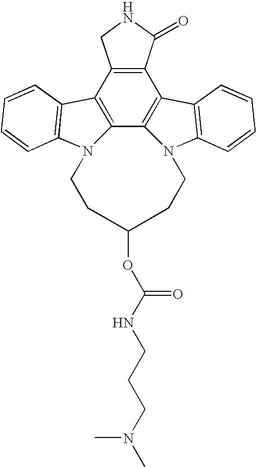 |
| Cpd. No. 187 | 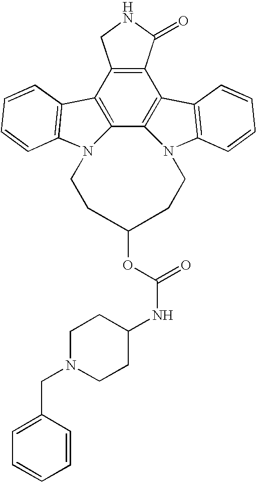 |
| Cpd. No. 188 | 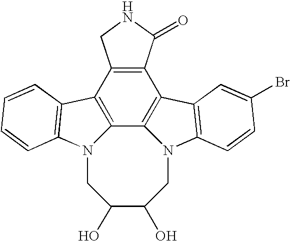 |
| Cpd. No. 189 | 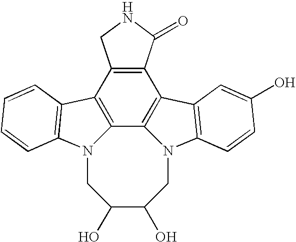 |
| Cpd. No. 190 | 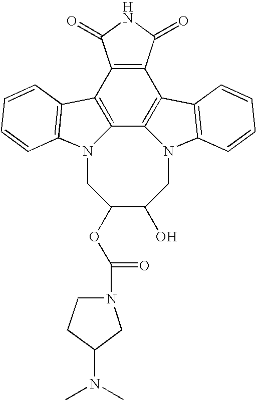 |
| Cpd. No. 191 | 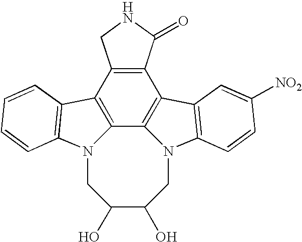 |
| Cpd. No. 192 | 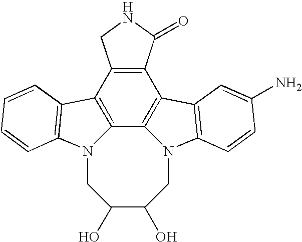 |
| Cpd. No. 193 | 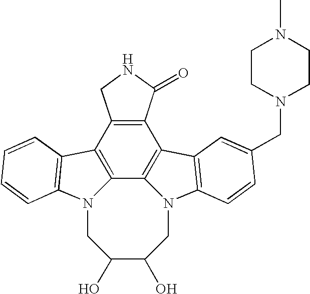 |
| Cpd. No. 194 | 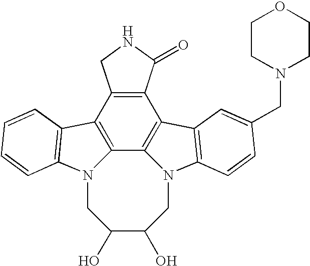 |
| Cpd. No. 195 | 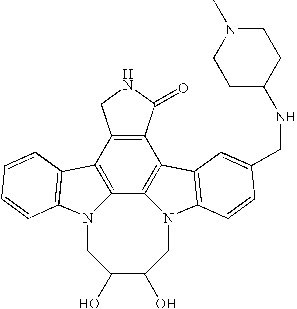 |
| Cpd. No. 196 | 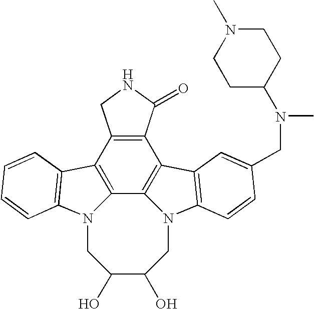 |
| Cpd. No. 197 | 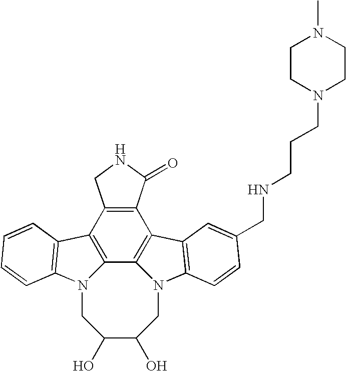 |
| Cpd. No. 198 | 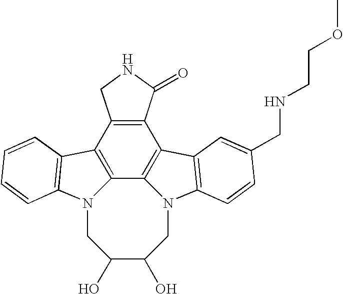 |
| Cpd. No. 199 | 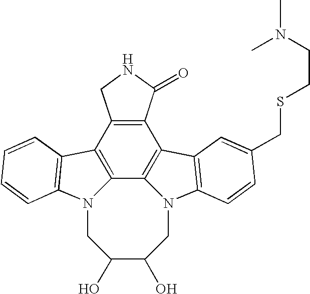 |
| Cpd. No. 200 | 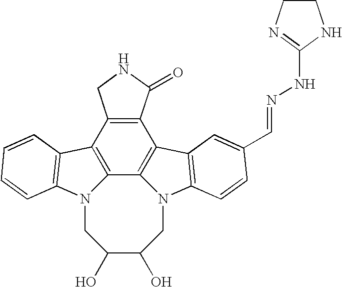 |
| Cpd. No. 201 | 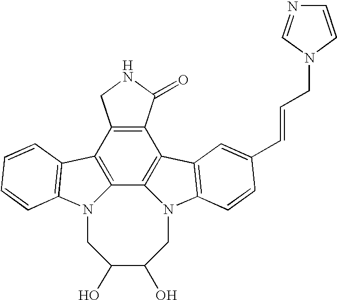 |
| Cpd. No. 202 | 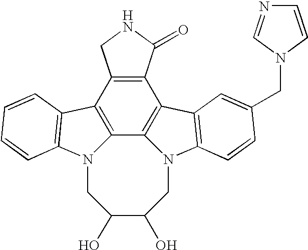 |
| Cpd. No. 203 | 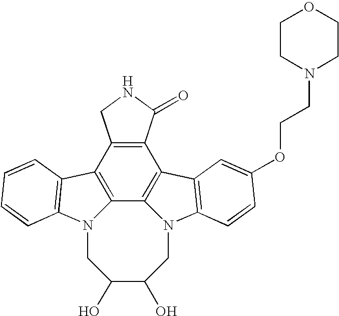 |
| Cpd. No. 204 | 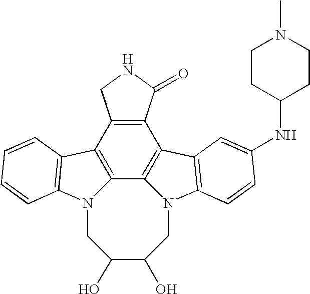 |
| Cpd. No. 205 | 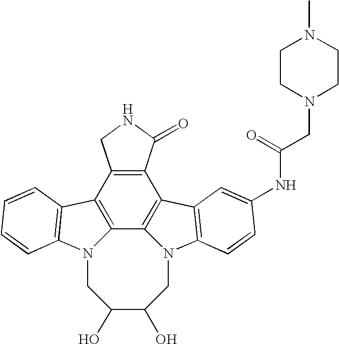 |
| Cpd. No. 206 | 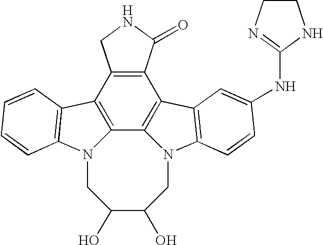 |
| Cpd. No. 207 | 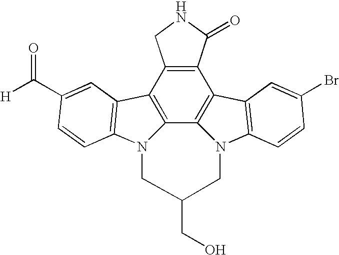 |
| Cpd. No. 208 | 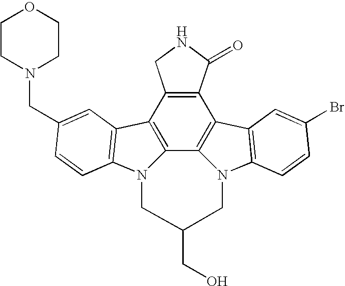 |
| Cpd. No. 209 | 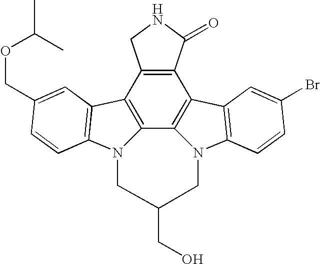 |
| Cpd. No. 210 | 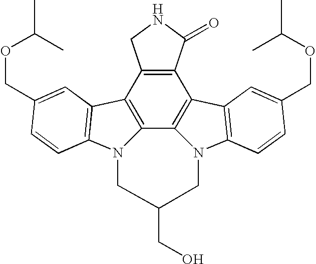 |
| Cpd. No. 211 | 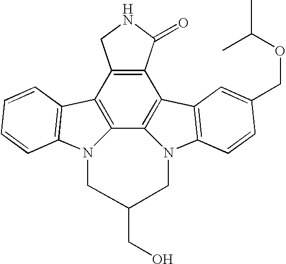 |
| Cpd. No. 212 | 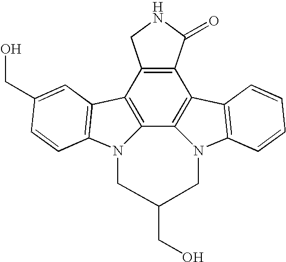 |
| Cpd. No. 213 | 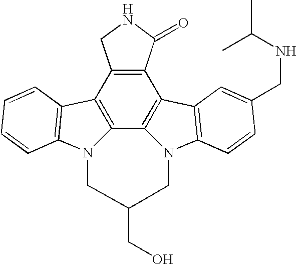 |
| Cpd. No. 214 | 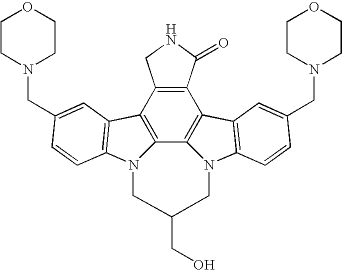 |
| Cpd. No. 215 | 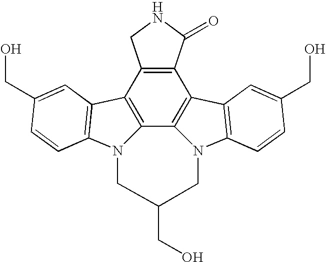 |
| Cpd. No. 216 | 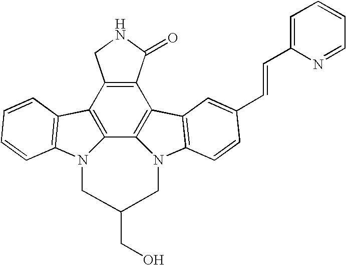 |
| Cpd. No. 217 | 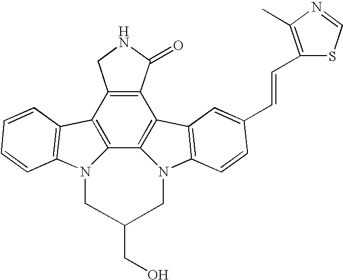 |
| Cpd. No. 218 | 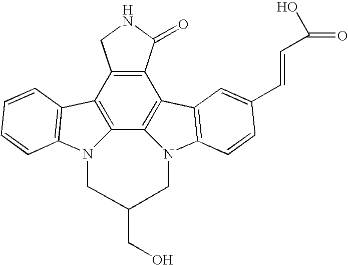 |
| Cpd. No. 219 | 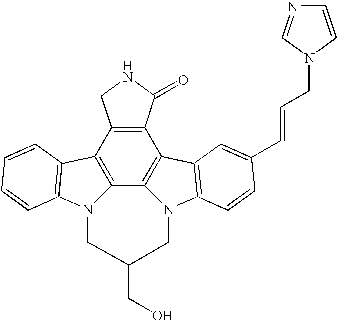 |
| Cpd. No. 220 | 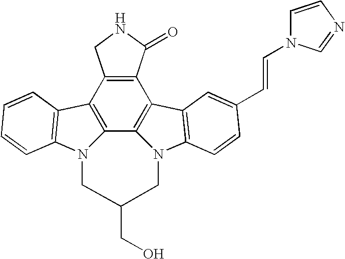 |
| Cpd. No. 221 | 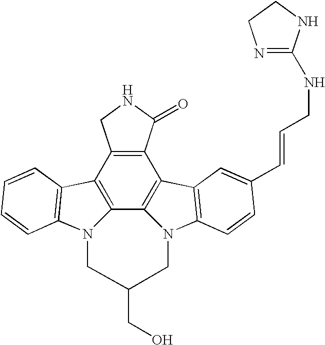 |
| Cpd. No. 222 | 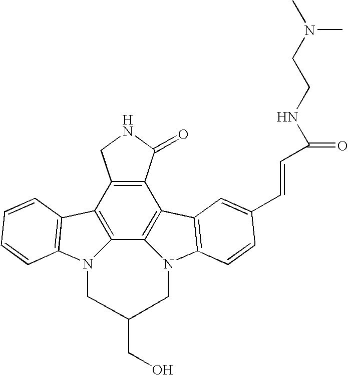 |
| Cpd. No. 223 | 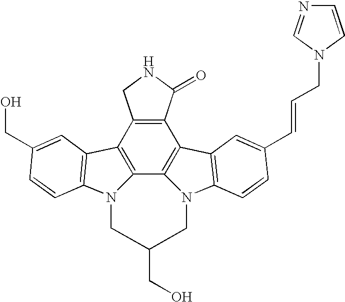 |

Chemical Definitions

As used herein, the following terms have the following meanings:

The term “C₁₋₈ alkyl,” whether used alone or as part of a substituent group, means a saturated branched or straight chain monovalent hydrocarbon radical or alkyldiyl linking group having a specified number of carbon atoms, wherein the radical is derived by the removal of one hydrogen atom from a single carbon atom and the alkyldiyl linking group is derived by the removal of one hydrogen atom from each of two carbon atoms in the chain. The term “C₁₋₈alkyl” refers to a radical having from 1-8 carbon atoms in a linear or branched arrangement.

Typical alkyl radicals include, but are not limited to, methyl, ethyl, 1-propyl, 2-propyl, 1-butyl, 2-butyl, tert-butyl, 1-pentyl, 2-pentyl, 3-pentyl, 1-hexyl, 2-hexyl, 3-hexyl, 1-heptyl, 2-heptyl, 3-heptyl, 1-octyl, 2-octyl, 3-octyl and the like. Embodiments include, e.g., the alkyl groups C₁₋₈alkyl or C₁₋₄ alkyl. Alkyl and alkyldiyl radicals may be attached to a core molecule via a terminal carbon atom or via a carbon atom within the chain. Similarly, any number of substituent variables may be attached to an alkyl or alkyldiyl radical when allowed by available valences.

The term “C₁₋₈ alkoxy,” whether used alone or as part of a substituent group, means an alkyl or alkyldiyl alcohol radical derived by the removal of the hydrogen atom from the hydroxide oxygen portion of the alcohol radical. Typical embodiments include, e.g., the alkoxy groups C₁₋₈alkoxy or C₁₋₄ alkoxy.

For example, “C₁₋₈ alkoxy” specifically includes the radicals methoxy, ethoxy, propoxy, butoxy, pentoxy, hexoxy, heptoxy, octoxy and the like. As described above, an alkoxy radical may be similarly attached to a core molecule and further substituted where indicated.

The term “C₃₋₈ cycloalkyl,” whether used alone or as part of a substituent group, means a saturated or partially unsaturated cyclic hydrocarbon ring system. Typical cycloalkyl radicals include, but are not limited to, cyclopropyl, cyclobutyl, cyclopentyl, cyclohexyl, indanyl, fluorenyl, acenaphthenyl and the like.

The term “heterocyclyl,” whether used alone or as part of a substituent group, means a saturated or partially unsaturated cyclic ring radical derived by the removal of one hydrogen atom from a single carbon atom of the ring system and in which one or more ring carbon atoms are a heteroatom selected from N, O, S, SO or SO₂. Embodiments include monocyclic or bicyclic rings wherein 1, 2, 3 or 4 members of the ring are a nitrogen atom, or 0, 1, 2 or 3 members of the ring are nitrogen atoms and 1 member is an oxygen or sulfur atom.

Typical heterocyclyl radicals include, and are not limited to, dihydro-1H-pyrrole (including 2-pyrrolinyl or 3-pyrrolinyl), pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, pyran, tetrahydropyranyl, tetrahydrothiopyranyl, piperidinyl, 1,4-dioxanyl, morpholinyl, 1,4-dithianyl, thiomorpholinyl, piperazinyl, azetidinyl, azepanyl, hexahydro-1,4-diazepinyl, hexahydro-1,4-oxazepanyl, tetrahydro-furyl, tetrahydro-thienyl, tetrahydro-pyranyl, tetrahydro-pyridazinyl, 1,3-benzodioxol-5-yl, 2,3-dihydro-1,4-benzodioxin-6-yl and the like.

The term “aryl,” whether used alone or as part of a substituent group, means an unsaturated cyclic ring radical derived by the removal of one hydrogen atom from a single carbon atom of the ring system. Typical aryl radicals include, and are not limited to, phenyl, naphthalenyl, indenyl, azulenyl, anthracenyl, biphenyl, benzhydryl and the like.

The term “heteroaryl,” whether used alone or as part of a substituent group, means an unsaturated cyclic ring radical derived by the removal of one hydrogen atom from a single carbon atom of the ring system and in which one or more ring carbon atoms are a heteroatom selected from N, O, S, SO or SO₂.

Typical heteroaryl radicals include, and are not limited to, furyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, indolizinyl, indolyl, isoindolyl, benzo[b]furyl, benzo[b]thienyl, indazolyl, benzimidazolyl, benzoxazolyl, benzthiazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl and the like.

The term C₁₋₈ acyl means a radical of the formula: —C(O)H or —C(O)—C₁₋₈alkyl, or a linking group of the formula: —C(O)—C₁₋₈ alkyl-terminal group.

The term C₁₋₈ alkoxy means a radical of the formula: —O—C₁₋₈ alkyl.

The term C₁₋₈ alkoxy-C₁₋₈ alkyl means a radical of the formula: —C₁₋₈alkyl-O—C₁₋₈alkyl, or a linking group of the formula: —C₁₋₈alkyl-O—C₁₋₈alkyl-terminal group.

The term C₁₋₈ alkoxy-amido means a radical of the formula: —NHC(O)—O—C₁₋₈ alkyl, or a linking group of the formula: —NHC(O)—O—C₁₋₈ alkyl-terminal group.

The term C₁₋₈ alkoxycarbonyl means a radical of the formula: —C(O)—O—C₁₋₈ alkyl, or a linking group of the formula: —C(O)—O—C₁₋₈ alkyl-terminal group.

The term C₁₋₈ alkyl-amino means a radical of the formula: —NH—C₁₋₈ alkyl or N(C₁₋₈ alkyl)₂.

The term C₁₋₈ alkyl-amino-C₁₋₈ alkyl means a radical of the formula: —C₁₋₈ alkyl-NH—C₁₋₈ alkyl or —C₁₋₈ alkyl-N(C₁₋₈ alkyl)₂, or a linking group of the formula: —C₁₋₈ alkyl-NH—C₁₋₈ alkyl-terminal group or —C₁₋₈ alkyl-N(C₁₋₈ alkyl)-C₁₋₈ alkyl-terminal group.

The term C₁₋₈ alkyl-amino-C₁₋₈ alkyl-carbamoyl-C₂₋₈ alkenyl means a C₁₋₈ alkyl-amino-C₁₋₈ alkyl radical or linking group substituted as the terminal group on a linking group of the formula: —C₂₋₈ alkenyl-C(O)NH-terminal group or —C₂₋₈ alkenyl-C(O)N(terminal group)₂.

The term C₁₋₈ alkyl-carbamoyl means a radical of the formula: —C(O)NH—C₁₋₈ alkyl or —C(O)N(C₁₋₈ alkyl)₂, or a linking group of the formula: —C(O)NH—C₁₋₈ alkyl-terminal group or —C(O)N(C₁₋₈ alkyl)-C₁₋₈ alkyl-terminal group.

The term C₁₋₈ alkyl-carbamoyl-C₂₋₈ alkenyl means a C₁₋₈ alkyl radical or linking group substituted as the terminal group on a linking group of the formula: —C₂₋₈ alkenyl-C(O)NH-terminal group or —C₂₋₈ alkenyl-C(O)N(terminal group)₂.

The term C₁₋₈ alkyl-carbamoyloxy means a radical of the formula: —O—C(O)NH—C₁₋₈ alkyl or —O—C(O)N(C₁₋₈ alkyl)₂, or a linking group of the formula: —O—C(O)NH—C₁₋₈ alkyl-terminal group or —O—C(O)N(C₁₋₈ alkyl)-C₁₋₈ alkyl-terminal group.

The term C₁₋₈ alkyl-carbamoyloxy-C₁₋₈ alkyl means a radical of the formula: —C₁₋₈ alkyl-O—C(O)NH—C₁₋₈ alkyl or —C₁₋₈ alkyl-O—C(O)N(C₁₋₈ alkyl)₂, or a linking group of the formula: —C₁₋₈ alkyl-O—C(O)NH—C₁₋₈ alkyl-terminal group or —C₁₋₈ alkyl-O—C(O)N(C₁₋₈ alkyl)-C₁₋₈ alkyl-terminal group.

The term C₁₋₈ alkylene means a radical of the formula: ═CH₂ (methylene), ═CH—C₁₋₈alkyl (substituted or unsubstituted methylidene) or ═C(C₁₋₈ alkyl)₂ (substituted or unsubstituted 1,1-bis(C₁₋₈ alkyl)methylidene)

The term amino means a radical of the formula: —NH₂.

The term amino-C₁₋₈ alkyl means a radical of the formula: —C₁₋₈ alkyl-NH₂, or a linking group of the formula: —C₁₋₈ alkyl-NH-terminal group or —C₁₋₈ alkyl-N(terminal group)₂.

The term amino-C₁₋₈ alkyl-carbamoyl-C₂₋₈ alkenyl means an amino-C₁₋₈ alkyl radical or linking group substituted as the terminal group on a linking group of the formula: —C₂₋₈ alkenyl-C(O)NH-terminal group or —C₂₋₈ alkenyl-C(O)N(terminal group)₂.

The term aminosulfonyl means a radical of the formula: —SO₂—NH₂.

The term (aryl)₂-C₁₋₈ alkyl means a radical such as substituted or unsubstituted benzhydryl.

The term carbamoyl means a radical of the formula: —C(O)NH₂, or a linking group of the formula: —C(O)NH-terminal group.

The term carbamoyloxy means a radical of the formula: —O—C(O)NH₂, or a linking group of the formula: —O—C(O)NH-terminal group.

The term carbamoyloxy-C₁₋₈ alkyl means a radical of the formula: —C₁₋₈ alkyl-O—C(O)NH₂, or a linking group of the formula: —C₁₋₈ alkyl-O—C(O)NH-terminal group.

The term carbonyloxy means a linking group of the formula: —O—C(O)-terminal group.

The term carboxy means a radical of the formula: —C(O)OH.

The term carboxy-C₂₋₈ alkenyl means a radical of the formula: —C₂₋₈ alkenyl-C(O)OH.

The term halogen means the group chloro, bromo, fluoro or iodo.

The term halo-C₁₋₈ alkoxy means a radical of the formula: —C₁₋₈ alkoxy(halo)₁₋₃ and includes monofluoromethoxy, difluoromethoxy, trifluoromethoxy, trifluoroethoxy and the like.

The term halo-C₁₋₈alkyl means a radical of the formula: —C₁₋₈alkyl(halo)₁₋₃ and includes monofluoromethyl, difluoromethyl, trifluoromethyl, trifluoroethyl and the like.

The term heterocyclyl-C₁₋₈ acyl means a radical of the formula: —C(O)—C₁₋₈alkyl-heterocyclyl.

The term heterocyclyl-C₁₋₈ acyl-amino means a radical of the formula: —NHC(O)—C₁₋₈ alkyl-heterocyclyl.

The term heterocyclyl-C₁₋₈ alkoxy means a radical of the formula: —O—C₁₋₈ alkyl-heterocyclyl.

The term heterocyclyl-amino means a radical of the formula: —NH-heterocyclyl.

The term heterocyclyl-amino-C₂₋₈ alkenyl means a radical of the formula: —C₂₋₈ alkenyl-NH-heterocyclyl.

The term heterocyclyl-carbonyl means a radical of the formula: —C(O)-heterocyclyl.

The term heterocyclyl-carbonyloxy means a radical of the formula: —O—C(O)-heterocyclyl.

The term heterocyclyl-carbonyloxy-C₁₋₈ alkyl means a radical of the formula: —C₁₋₈ alkyl-O—C(O)-heterocyclyl.

The term hydroxy-C₁₋₈ alkyl means a radical wherein C₁₋₈ alkyl is substituted on an available carbon chain atom with one or more hydroxy radicals.

The term hydroxy-C₁₋₈ alkoxy means a radical wherein C₁₋₈ alkoxy is substituted on an available carbon chain atom with one or more hydroxy radicals.

The term imino-C₁₋₈ alkyl means a radical of the formula: —C₁₋₈ alkyl=NH, or a linking group of the formula: —C₁₋₈ alkyl=N-terminal group.

The term thio-C₁₋₈ alkyl means a linking group of the formula: —C₁₋₈ alkyl-5-terminal group.

The term “substituted” means the independent replacement of one or more hydrogen atoms within a radical with that amount of substituents allowed by available valences.

The term “dependently substituted” means that the structure variables are specified in an indicated combination.

Chemical Nomenclature

In general, IUPAC nomenclature conventions are used throughout this disclosure. In certain instances, the following rules apply to the nomenclature used to describe compounds of the present invention:

In reference to a core molecule of formula (I), the following names are used:

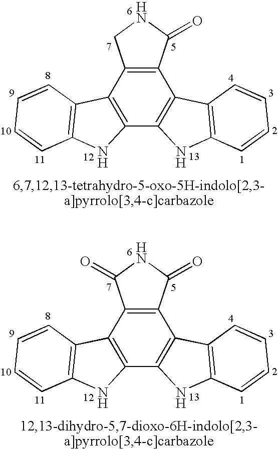

In reference to a core molecule of formula (I) bridged at the 12,13 position with an alkyl chain, the following names are used:

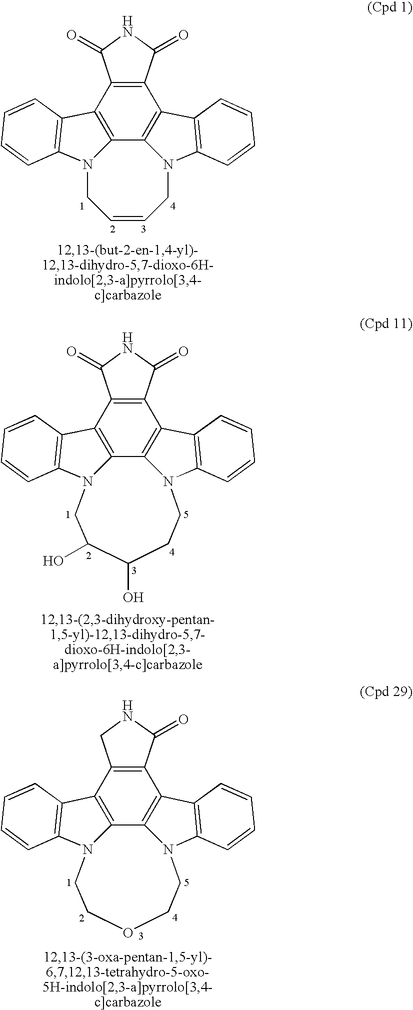

In reference to a core molecule of formula (I) with a ring fused in or attached to the alkyl chain bridged at the 12,13 position, the following names are used: 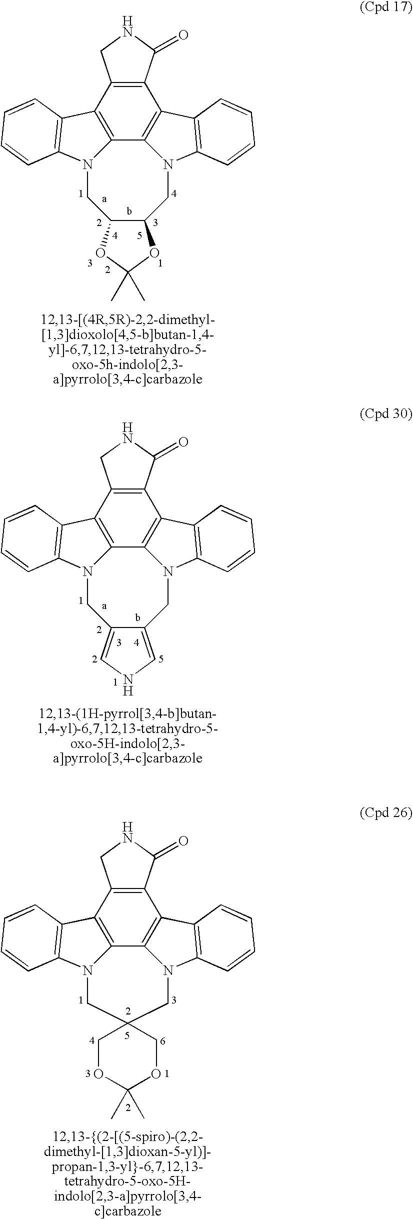
Compound Forms

The term “forms” and “forms thereof” means that the compounds of the present invention may exist in various salt, stereoisomer, crystalline, solvate, ester, prodrug or active metabolite forms. The present invention encompasses all such compound forms, including active compounds in the form of essentially pure enantiomers, racemic mixtures and tautomers.

The compounds of the invention may be present in the form of pharmaceutically acceptable salts. For use in medicines, the “pharmaceutically acceptable salts” of the compounds of this invention refer to non-toxic acidic/anionic or basic/cationic salt forms.

Pharmaceutically acceptable acidic/anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide salts.

Organic or inorganic acids also include, and are not limited to, hydroiodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol, ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate, sodium hydroxide, triethanolamine or zinc.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3^{rd} Edition, John Wiley & Sons, 1999. The protecting groups may be removed at a convenient subsequent stage using methods known in the art.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (stereoisomers).

The term “stereoisomer” means isomers of identical constitution that differ in the spatial arrangement of their atoms. Enantiomers and diastereomers are stereoisomers wherein an asymmetrically substituted carbon atom acts as a chiral center. The term “chiral” means a molecule that is not superimposable on its mirror image, implying the absence of an axis and a plane or center of symmetry. The term “enantiomer” means one of a pair of molecular species that are mirror images of each other and are not superimposable. The term “diastereomer” means stereoisomers that are not related as mirror images. The symbols “R” and “S” represent the configuration of substituents around a chiral carbon atom(s).

The term “racemate” or “racemic mixture” means a compound of equimolar quantities of two enantiomeric species, wherein the compound is devoid of optical activity. The term “optical activity” means the degree to which a chiral molecule or non-racemic mixture of chiral molecules rotates the plane of polarized light.

“Geometric isomer” means isomers that differ in the orientation of substituent atoms in relationship to a carbon-carbon double bond, to a cycloalkyl ring, or to a bridged bicyclic system. Substituent atoms (other than H) on each side of a carbon-carbon double bond may be in an E or Z configuration. In the “E” configuration, the substituents are on opposite sides in relationship to the carbon-carbon double bond. In the “Z” configuration, the substituents are oriented on the same side in relationship to the carbon-carbon double bond.

The isomeric descriptors (“R,” “S,” “E,” and “Z”) indicate atom configurations relative to a core molecule and are intended to be used as defined in the literature.

The compounds of the invention may be prepared as individual isomers by either isomer-specific synthesis or resolved from an isomeric mixture. Conventional resolution techniques include combining the free base (or free acid) of each isomer of an isomeric pair using an optically active acid (or base) to form an optically active salt (followed by fractional crystallization and regeneration of the free base), forming an ester or amide of each of the isomers of an isomeric pair by reaction with an appropriate chiral auxiliary (followed by fractional crystallization or chromatographic separation and removal of the chiral auxiliary), or separating an isomeric mixture of either an intermediate or a final product using various well known chromatographic methods.

Furthermore, compounds of the invention may have one or more polymorph or amorphous crystalline forms. Said forms are included in the scope of the invention. In addition, some of the compounds may form solvates with water (i.e., hydrates) or common organic solvents. Said solvates are encompassed within the scope of this invention.

Methods of Use

The present invention includes a method for inhibiting unregulated protein kinase activity comprising contacting a protein kinase domain with one or more compounds of formula (I).

An aspect of this method includes inhibiting unregulated JAK3 protein kinase activity.

Another aspect of this method includes inhibiting increased or unregulated JAK3 mediated cytokine expression, signaling or migration, whereby such expression, signaling or migration results in an inflammatory response or an immunodeficiency.

The present invention also includes a method for use of one or more compounds of formula (I) as a medicine or therapeutic agent for treating, preventing or ameliorating a chronic or acute protein kinase mediated disease, disorder or condition in a subject in need thereof comprising administering to the subject an effective amount of one or more compounds of formula (I) or a pharmaceutical composition or medicament thereof.

An aspect of this method includes treating, preventing or ameliorating a chronic or acute JAK3 mediated disease, disorder or condition associated with increased or unregulated cytokine expression, signaling or migration and the like in the subject.

An aspect of this method includes administering to the subject an effective amount of a compound of formula (I) or pharmaceutical composition thereof in the form of a medicine or medicament. Consequently, the invention encompasses the use of the compound of formula (I) as a medicine or medicament.

The present invention includes the use of a compound of formula (I) for the manufacture of a medicine or medicament for treating, preventing or ameliorating a chronic or acute JAK3 mediated disease, disorder or condition.

Accordingly, the present invention is directed to a method for treating, preventing or ameliorating a chronic or acute protein kinase mediated disease, disorder or condition in a subject in need thereof comprising administering to the subject an effective amount of one or more compounds of formula (I) or a pharmaceutical composition thereof.

The term “chronic or acute protein kinase mediated disease, disorder or condition” includes, and is not limited to diseases, disorders or conditions associated with increased or unregulated JAK3 mediated cytokine expression, signaling or migration, whereby such expression, signaling or migration results in an inflammatory response or an immunodeficiency.

The term “increased or unregulated cytokine expression, signaling or migration” refers to 1) increased or unregulated cytokine expression, signaling or migration, 2) increased cytokine expression, signaling or migration leading to an inflammatory response or an immunodeficiency, 3) increased kinase signaling leading to increased or unregulated cytokine expression, signaling or migration, or 4) mutations leading to constitutive kinase activation, whereby such activation results in an inflammatory response or an immunodeficiency.

The existence of increased or unregulated cytokine expression, signaling or migration may be determined by procedures well known in the art.

The term “expression, signaling or migration” refers to cytokine expression, signaling or migration from one or more subset of cells in a multicellular organism resulting in harm (such as discomfort or decreased life expectancy) to the multicellular organism.

The term “treating, preventing or ameliorating” includes, and is not limited to, facilitating the eradication of, inhibiting the progression of or promoting stasis of an inflammatory response or an immunodeficiency.

The foregoing methods contemplate that the compounds of the present invention are therapeutically useful for treating, preventing or ameliorating JAK3 mediated diseases, disorders or conditions such as, without limitation, transplantation rejection, psoriasis, psoriatic arthritis, graft-versus-host disease, multiple sclerosis, inflammatory bowel disease, systemic lupus erythematosus, rheumatoid arthritis, allergic diseases or asthma.

The term “administering,” with respect to the methods of the present invention, refers to a means for treating, ameliorating or preventing a disease, disorder or condition as described herein with a compound specifically disclosed or a compound or prodrug thereof, which would obviously be included within the scope of the invention albeit not specifically disclosed for certain of the instant compounds.

Such methods include prophylactically or therapeutically administering an effective amount of one or more compounds of formula (I) or a composition or medicament thereof at different times during the course of a therapy or concurrently in a combination form. Prophylactic administration can occur prior to the manifestation of symptoms characteristic of a kinase associated disease or disorder such that the disease or disorder is prevented or, alternatively, delayed in its progression. The instant invention is therefore to be understood as embracing all such regimes of simultaneous or alternating treatment and the term “administering” is to be interpreted accordingly.

The term “prodrug” refers to a metabolic precursor of a compound of formula (I) or pharmaceutically acceptable form thereof. In general, a prodrug is a functional derivative of a compound, which may be inactive when administered to a subject, but is readily convertible in vivo into an active metabolite compound.

The term “active metabolite” refers to a metabolic product of a compound that is pharmaceutically acceptable and effective. Conventional procedures for the selection and preparation of suitable prodrug derivatives are described, for example, in “Design of Prodrugs”, ed. H. Bundgaard, Elsevier, 1985.

The term “subject” as used herein, refers to a patient, such as an animal, mammal or human, who has been the object of treatment, observation or experiment and is at risk of (or susceptible to) developing a disease, disorder or condition or having a disease, disorder or condition related to increased or unregulated cytokine expression, signaling or migration.

The term “effective amount” refers to that amount of active compound or pharmaceutical agent that elicits the biological or medicinal response (such as inhibiting activation of unregulated kinase activity) in a tissue system, animal or human, that is being sought by a researcher, veterinarian, medical doctor, or other clinician, which includes treating, preventing or ameliorating the symptoms of the disease, disorder or condition being treated.

An example of the effective amount of a compound of formula (I) exemplified in such a method is from about 0.001 mg/kg/day to about 300 mg/kg/day

Another example of the effective amount for an instant compound is a compound of formula (I) having an IC₅₀ (50% inhibition concentration) binding activity against JAK3 in a range of about 50 μM or less, of about 25 μM or less, of about 10 μM or less, of about 1 μM or less, of about 0.5 μM or less, of about 0.25 μM or less, of about 0.1 μM or less, or of about 0.05 μM or less.

The term “composition” refers to a product containing a compound of the present invention, wherein the product comprises the specified ingredients in the specified amounts, as well as any product which results, directly or indirectly, from such combinations of the specified ingredients in the specified amounts.

The term “medicament” refers to a product for use in treating, preventing or ameliorating a JAK3 mediated disease, disorder or condition.

The term “pharmaceutically acceptable” refers to molecular entities and compositions that are of sufficient purity and quality for use in the formulation of a composition or medicament of the present invention and that, when appropriately administered to an animal or a human, do not produce an adverse, allergic or other untoward reaction. Since both human use (clinical and over-the-counter) and veterinary use are equally included within the scope of the present invention, a pharmaceutically acceptable formulation would include a composition or medicament for either human or veterinary use.

The methods of the present invention further include administering to the subject an effective amount of a combination product comprising one or more compounds of formula (I) or a composition or medicament thereof and at least one other therapeutic agent at different times during the course of a therapy or concurrently as a combination product.

Such a combination product may advantageously facilitate administering to the subject an amount of an agent or a compound of formula (I) that is either or both reduced relative to the amount which would be given in the absence of the other.

Therefore, it is contemplated that the compounds of this invention can be administered to the subject before, during or after the time a particular therapeutic agent is administered

The term “therapeutic agent” includes, and is not limited to, anti-inflammatory agents, immunosuppressive agents and the like.

The term “combination therapy” refers to the use of one or more compounds of formula (I) or composition or medicament thereof advantageously administered in one or more anti-inflammatory or immunosuppressive therapies or as an adjunct to such therapy for treating, preventing or ameliorating a chronic or acute JAK3 mediated disease, disorder or condition.

The combination therapy comprises
1. coadministration of a compound of formula (I) or pharmaceutical composition thereof and a therapeutic agent,
2. sequential administration of a compound of formula (I) or pharmaceutical composition thereof and a therapeutic agent,
3. administration of a pharmaceutical composition containing a compound of formula (I) or pharmaceutical composition thereof and a therapeutic agent, or,
4. simultaneous administration of a separate pharmaceutical composition containing a compound of formula (I) or pharmaceutical composition thereof and a separate pharmaceutical composition containing a therapeutic agent.

Each agent is administered in an effective amount, which varies based on the agent used, the type of inflammation to be treated or ameliorated and other conditions according to methods well known in the art.

As will be understood by those of ordinary skill in the art, the appropriate doses of therapeutic agents will be generally around those already employed in clinical therapies wherein the therapeutic agents are administered alone or in combination with other therapeutic agents.

The present invention further includes a method for use of a compound of formula (I) as a marker, wherein the compound is labeled with a ligand such as a radioligand (selected from deuterium, tritium and the like).

Pharmaceutical Compositions

An example of the present invention includes a pharmaceutical composition comprising an admixture of one or more compounds of formula (I) and/or one or more pharmaceutically acceptable forms thereof and one or more pharmaceutically acceptable excipients.

The pharmaceutically acceptable forms for a compound of formula (I) include a pharmaceutically acceptable salt, ester, prodrug or active metabolite of a compound of formula (I).

Pharmaceutical compositions according to the invention may, alternatively or in addition to a compound of formula I, comprise as an active ingredient a pharmaceutically acceptable salt of a compound of formula I or a prodrug or pharmaceutically active metabolite of such a compound or salt.

The present invention further includes the use of a process for making the composition or medicament comprising mixing one or more of the instant compounds and an optional pharmaceutically acceptable carrier; and, includes those compositions or medicaments resulting from such a process. Contemplated processes include both conventional and unconventional pharmaceutical techniques.

The composition or medicament may take a wide variety of forms to effectuate mode of administration, including, but not limited to, intravenous (both bolus and infusion), oral, nasal, transdermal, topical with or without occlusion, and injection intraperitoneally, subcutaneously, intramuscularly, intratumorally or parenterally. The composition or medicament may be in a dosage unit such as a tablet, pill, capsule, powder, granule, sterile parenteral solution or suspension, metered aerosol or liquid spray, drop, ampoule, auto-injector device or suppository; for administration orally, parenterally, intranasally, sublingually or rectally or by inhalation or insufflation.

Compositions or medicaments suitable for oral administration include solid forms such as pills, tablets, caplets, capsules (each including immediate release, timed release and sustained release formulations), granules and powders; and, liquid forms such as solutions, syrups, elixirs, emulsions and suspensions. Forms useful for parenteral administration include sterile solutions, emulsions and suspensions. Furthermore, compositions or medicaments can be administered in intranasal form via topical use of suitable intranasal vehicles, or via transdermal routes, using, e.g., those forms of transdermal skin patches well known to those of ordinary skill in that art.

The compounds of the present invention can also be administered in the form of liposome or otherwise encapsulated delivery systems, such as small unilamellar vesicles, large unilamellar vesicles and multilamellar vesicles. Liposomal delivery systems are well known in the art and are formed from a variety of phospholipids, such as cholesterol, stearylamine, phosphatidylcholine and the like.

Advantageously, a compound of formula (I) may be administered in a single daily dose, or the total daily dosage may be administered in divided doses of two, three or four times daily. Alternatively, the composition or medicament may be presented in a form suitable for once-weekly or once-monthly administration; for example, an insoluble salt of the active compound, such as the decanoate salt, may be adapted to provide a depot preparation for intramuscular injection.

The dosage form (tablet, capsule, powder, injection, suppository, teaspoonful and the like) containing the composition or medicament contains an effective amount of the active ingredient necessary to be therapeutically or prophylactically effective as described above.

The composition or medicament may contain from about 0.001 mg to about 5000 mg (preferably, from about 0.001 to about 500 mg) of the active compound or prodrug thereof and may be constituted into any form suitable for the mode of administration selected for a subject in need. A contemplated effective amount may range from about 0.001 mg to about 300 mg/kg of body weight per day. Preferably, the range is from about 0.003 to about 100 mg/kg of body weight per day. Most preferably, the range is from about 0.005 to about 15 mg/kg of body weight per day. The composition or medicament may be administered according to a dosage regimen of from about 1 to about 5 times per day.

For oral administration, the composition or medicament is preferably in the form of a tablet or capsule containing, e.g., 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the subject to be treated.

Optimal dosages will vary depending on factors associated with the particular subject being treated (e.g., age, weight, diet and time of administration), the severity of the condition being treated, the compound being employed, the mode of administration and the strength of the preparation. The use of either daily administration or post-periodic dosing may be employed.

Synthetic Methods

Representative compounds of the present invention can be synthesized in accordance with the general synthetic schemes described below and are illustrated more particularly in the specific synthetic examples that follow. The general schemes and specific examples are offered by way of illustration; the invention should not be construed as being limited by the chemical reactions and conditions expressed. Except where indicated, starting materials and intermediates used in the schemes and examples are prepared by known methodologies well within the ordinary skill of persons versed in the art. No attempt has been made to optimize the yields obtained in any of the example reactions. One skilled in the art would also know how to increase such yields through routine variations in materials, solvents, reagents, reaction conditions and the like. All commercially available chemicals were used without further purification. Particular equipment components used in the examples such as reaction vessels and the like are also commercially available.

The terms used in describing the invention are commonly used and known to those skilled in the art. When used herein, the following abbreviations have the indicated meanings:

Boc tert-butoxycarbonyl; tert-butyl ester

CDI 1,1′-carbonyl diimidazole

Cpd compound

9-BBN 9-borabicyclo[3.3.1]nonane

DCM dichloromethane

DDQ 2,3-dichloro-5,6-dicyanoquinone

DMAP 4-(dimethylamino)-pyridine

DMF N,N-dimethylformamide

DMSO dimethyl sulfoxide

EtOAc ethyl acetate

hr(s)/min(s) hour(s)/min(s)

LiOH lithium hydroxide

MeOH methanol

MTBD 7-methyl-1,5,7-triazabicyclo-(4.4.0)-dec-5-ene

NBS N-bromosuccinimide

RT/rt/r.t. room temperature

TFA trifluoroacetic acid

THF tetrahydrofuran

NaHCO₃ sodium bicarbonate

NH₄Cl ammonium chloride

NH₄OH ammonium hydroxide

NaOH sodium hydroxide

General Synthetic Methods

Representative compounds of the present invention can be synthesized in accordance with the general synthetic methods described below, which are illustrated more particularly in the schemes that follow. The invention should not be construed as being limited by the chemical reactions and conditions expressed.

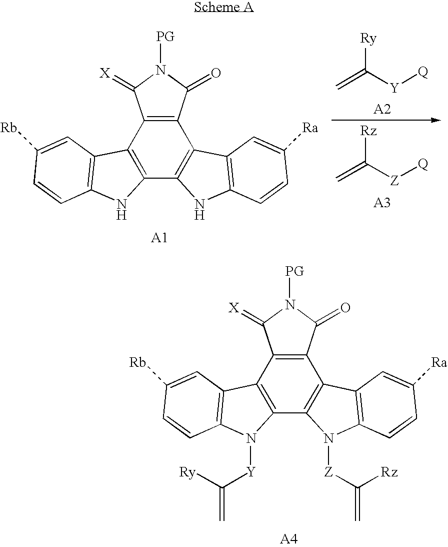

A solution of a Compound A1 (wherein Ra and Rb may be present as hydrogen or added as functional groups and PG represents a suitable protecting group such as alkyl, Boc, Fmoc and the like) is reacted either simultaneously or sequentially with a Compound A2 and a Compound A3 (wherein Q is a suitable leaving group such as a halogen and the like, Y and Z are as defined herein and Ry and Rz represent appropriate sub stituents as defined herein by W) in the presence of a suitable reagent (such as Cs₂CO₃ and the like) in a suitable solvent (such as DMF, CH₃CN and the like) at a suitable temperature to provide a Compound A4.

When reacted simultaneously, Compound A2 and Compound A3 are equivalent (i.e., Y. Z. Ry and Rz are the same). When reacted sequentially, Compound A1 and Compound A3 are not equivalent (i.e., one or more of Y. Z. Ry and Rz are not the same).

Ra and Rb may be added as functional groups via substitution reactions using conditions and techniques (e.g., brominations, formylations, nitrations, palladium couplings, reductive aminations, reductions, oxidations, alkylations and the like) known to those skilled in the art to provide compounds representative of the present invention

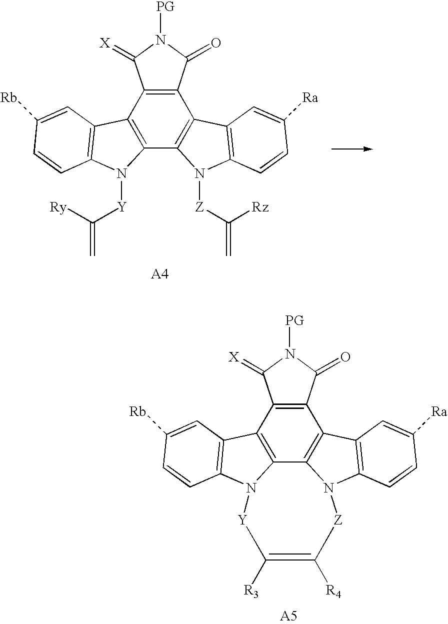

Compound A4 is reacted with a suitable Grubbs I (first generation) or Grubbs II (second generation) metalated coupling reagent (such as, respectively, benzylidene-bis(tricyclohexylphosphine)dichlororuthenium, 1,3-bis-[(2,4,6-trimethylphenyl)-2-imidazolidinylidene]dichloro(phenylmethylene)-(tricyclohexylphosphine)ruthenium and the like) in a suitable solvent (such as DCM, 1,2-dichloroethane and the like) at a suitable temperature to provide a compound of formula (I) selected from Compound A5, wherein W is —C(R₃)═C(R₄)—.

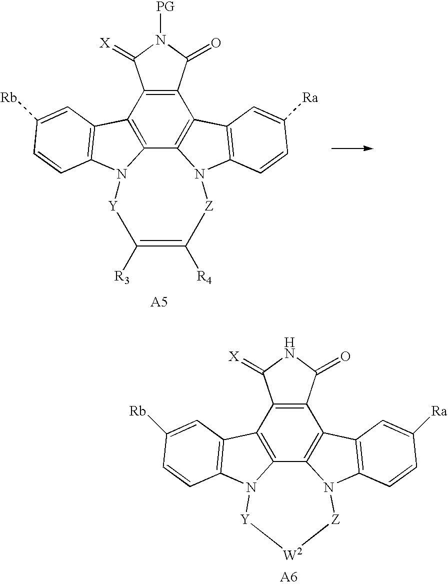

Compound A5 is reacted with a solution of a suitable acid or base for selective removal of protecting groups to provide an intermediate which is reacted with a solution of a suitable reagent or a mixture thereof (such as OsCl₃, BH₃ and the like) in a suitable solvent (such as THF and the like) at a suitable temperature to provide a compound of formula (I) selected from Compound A6, wherein W² is —C(R¹,R₁ₐ)—C(R₂,R₂ₐ)—, —C(R₅,R₅ₐ)— or —C(R₆)—.

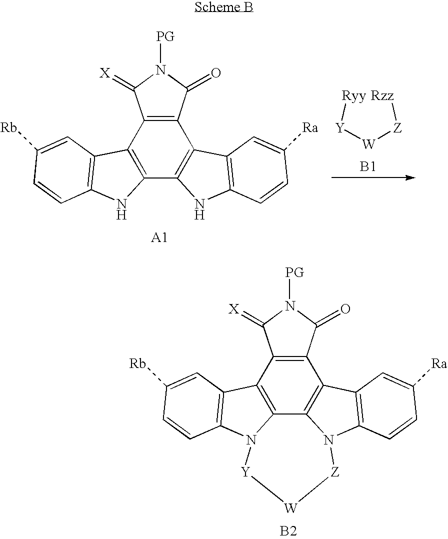

A solution of a Compound A1 is reacted with at least one or up to several equivalents of a Compound B1 (wherein Ryy and Rzz are suitable leaving groups such as a tosyl, a halogen and the like) in the presence of a suitable reagent at a suitable temperature to provide a Compound B2, which is carried forward according to the procedure of Scheme A to provide a compound of formula (I).

### Example 1

12,13-(2,3-dihydroxy-butan-1,4-yl)-6,7,12,13-tetrahydro-5-oxo-5H-indolo[2,3-a]pyrrolo[3,4-c]carbazole (Compound 14)

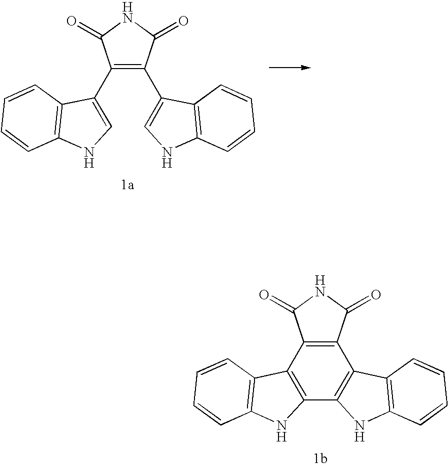

Palladium dichloride (7.4 g, 41.6 mmoles) was added to a solution of acryrubin A Compound 1a (2.9 g, 8.86 mmol) (prepared as described in Faul M M, Winneroski L L and Krumrich C A, Journal of Organic Chemistry, 1998, 63, 6053-6058) in DMF (100 mL) at 90° C. The reaction temperature was kept at 90° C. for 1 hr. The mixture was cooled and conc. HCl (50 mL), then water (50 mL) was added. The mixture was poured over ice and the resulting precipitate was filtered off. The solids were washed with H₂O and MeOH, then dissolved in THF (200 mL) and acetone (200 mL) and the remaining solids were filtered off. The solution was filtered through a plug of silica gel and the solvent was removed under vacuum. The resulting residue was diluted with MeOH, the solids were filtered and washed with MeOH then dried to provide acryflavin A Compound 1b (2 g, 70%) as a brown solid.

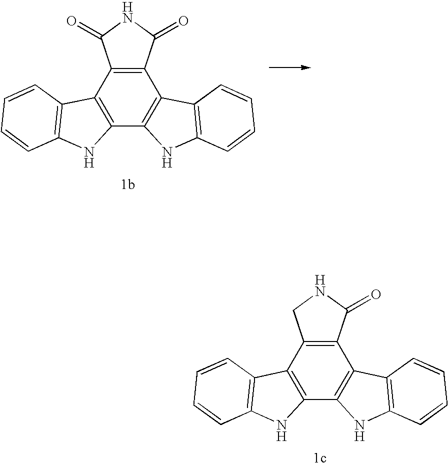

HCl (conc.) (45 mL) was added dropwise over a 2 hr period to a solution of Compound 1b (2 g, 6.15 mmoles) and tin (18 g) in acetic acid (100 mL) at 100° C. The mixture was heated for an additional 2 hrs at 100° C., and THF (75 mL) was added. The mixture was cooled to room temperature, then filtered over celite and the filter cake washed with THF (200 mL) and acetone (200 mL). The washings were combined and poured over ice. The resulting precipitate was filtered and washed sequentially with H₂O and Et₂O (4 times each), then dried to provide To a microwave tube was added (also referred to as K-252c) (1.27 g) as a light brown solid. The filter cake was rewashed with additional portions of MeOH (200 mL), THF (100 mL) and acetone (100 mL). The solvent was removed from the combined additional washings and the resulting residue was diluted with Et₂O and filtered to provide additional Compound 1c (0.37 g) (combined yield 1.64 g, 86%). ¹H NMR (300 MHz, d⁶-DMSO): δ 4.97 (s, 2H), 7.24 (t, 1H, J=7.5 z), 7.32 (t, 1H, J=7.8 Hz), 7.44 (quin, 2H, J=8.4 Hz), 7.73 (d, 1H, J=8.1 Hz), 7.8 (d, 1H, J=8.1 Hz), 8.05 (d, 1H, J=7.8 Hz), 8.47 (s, 1H), 9.23 (d, 1H, J=7.8 Hz), 11.35 (s, 1H), 11.52 (s, 1H); MS m/z 645 (2M+Na), 334 (M+Na), 312 (M+H).

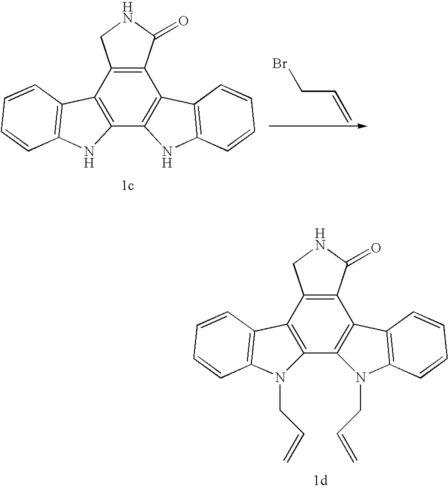

Cs₂CO₃ (2.5 g) was added to a solution of Compound 1c (650 mg, 2.09 mmoles) in DMF (30 mL). The mixture was stirred for 1 hr at r.t., then 3-bromo-propene (1 mL) was added. The mixture was stirred for 6 hrs, then additional 3-bromo-propene (1 mL) was added. The mixture was stirred for overnight at r.t., then the reaction mixture was extracted with EtOAc and sequentially washed with NH₄Cl (aq.) and NaCl (aq.). The organic layers were separated and dried over Na₂SO₄(s), then filtered and the solvent was removed. The resulting brown residue was diluted with Et₂O and the solids were filtered off, then washed with Et₂O and dried to provide 12,13-diallyl-6,7,12,13-tetrahydro-5-oxo-5H-indolo[2,3-a]pyrrolo[3,4-c]carbazole Compound 1d (584 mg, 72%) as a yellowish-brown solid. ¹H NMR (300 MHz, CDCl₃): δ 4.95 (m, 4H), 5.48 (m, 4H), 6.2 (m, 2H), 7.43 (m, 2H), 7.55 (m, 4H), 7.92 (d, 1H, J=7.8 Hz), 9.57 (d, 1H, J=7.8 Hz); MS m/z 805 (2M+Na), 783 (2M+H), 392 (M+H).

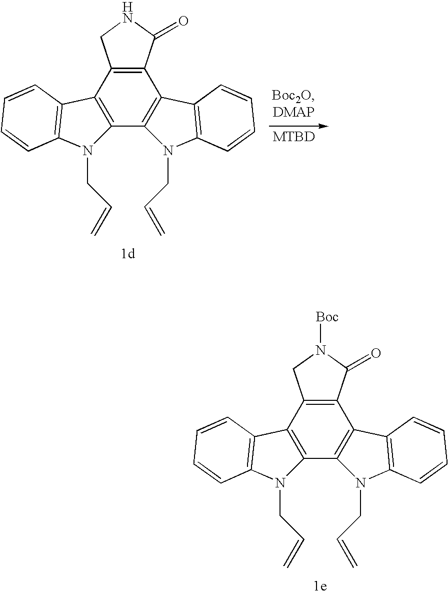

Compound 1d (615 mg, 1.57 mmoles), di-t-butoxy-carbonyl anhydride (450 mg) and DMAP (250 mg) were dissolved in THF (20 mL). MTBD (0.23 mL) was added and the mixture was stirred at r.t. for 2 hrs. Additional di-t-butoxy-carbonyl anhydride (400 mg) was added and the mixture was stirred for 2 hrs. The reaction was quenched with NH₄Cl(aq.), then extracted with EtOAc and washed with a solution of NaCl (aq.). The aqueous layers were reextracted, the organic layers were combined and dried over Na₂SO₄(s), then filtered and the solvents removed. The resulting residue was triturated with ethyl ether and filtered, then washed with ether and dried to provide 12,13-diallyl-6,7,12,13-tetrahydro-5-oxo-5H-indolo[2,3-a]pyrrolo[3,4-c]carbazole-6-carboxylic acid tert-butyl ester Compound 1e (644 mg, 83%) as a brown-orange solid. ¹H NMR (300 MHz, CDCl₃): δ 1.74 (s, 9H), 4.95 (m, 2H), 5.05 (m, 2H), 5.28 (s, 2H), 5.48 (m, 4H), 6.19 (m, 2H), 7.42 (m, 2H), 7.51 (m, 2H), 7.56 (m, 2H), 7.99 (d, 1H, J=8.8 Hz), 9.55 (d, 1H, J=7.9 Hz); MS m/z 1005 (2M+Na), 514 (M+Na).

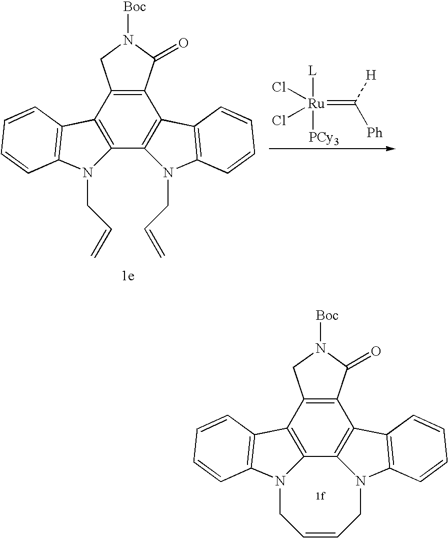

Benzylidene-bis(tricyclohexylphosphine)dichlororuthenium (125 mg) was added to a solution of Compound 1e (350 mg, 0.71 mmoles) in DCM (70 mL). The reaction was stirred at r.t. for 20 hrs, then filtered over silica gel with excess DCM. The DCM layers were discarded and the silica gel washed with EtOAc. The EtOAc layers were removed under vacuum, the resulting residue was diluted withh ethyl ether and the solids were filtered and dried to provide 12,13-(but-2-en-1,4-yl)-6,7,12,13-tetrahydro-5-oxo-5H-indolo[2,3-a]pyrrolo[3,4-c]carbazole-6-carboxylic acid tert-butyl ester Compound 1f (282 mg, 85%) as a gray solid. ¹H NMR (300 MHz, CDCl₃): δ 1.75 (s, 9H), 4.19 (d, 2H, J=7.2 Hz), 4.23 (s, 2H), 4.95 (d, 2H, J=5.1 Hz), 6.08 (m, 2H), 7.31 (m, 1H), 7.39 (m, 2H), 7.57 (m, 3H), 7.66 (d, 1H, J=7.2 Hz), 9.72 (d, 1H, J=8.1 Hz); MS m/z 949 (2M+Na).

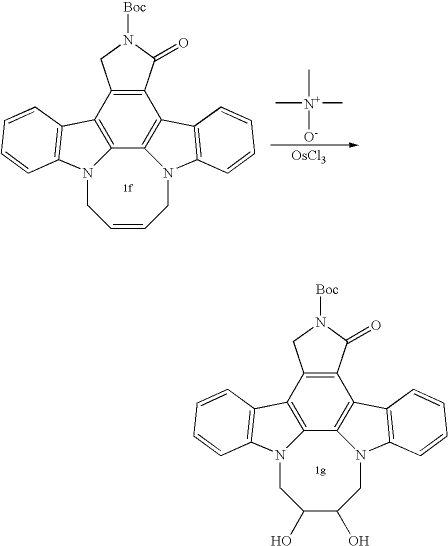

Compound 1f (162 mg, 0.35 mmoles) and trimethylamine-N-oxide (155 mg) were dissolved in chloroform (15 mL) and THF (15 mL), then water (10 drops) and osmium trichloride (9 mg) were added. The mixture was stirred for 4 hrs at r.t., then the solvent was removed under vacuum and water was added. The resulting solids were filtered, sequentially washed with water and ethyl ether (4 times each) and dried to provide 12,13-(2,3-dihydroxy-butan-1,4-yl)-6,7,12,13-tetrahydro-5-oxo-5H-indolo[2,3-a]pyrrolo[3,4-c]carbazole-6-carboxylic acid tert-butyl ester Compound 1g (158 mg, 91%) as a gray solid. ¹H NMR (300 MHz, d⁶-DMSO): δ 1.64 (s, 9H), 4.28 (d, 2H, J=3.3 Hz), 4.62 (m, 3H), 4.73 (m, 1H), 5.27 (quart, 2H, J=17.1 Hz), 5.43 (s, 2H), 7.32 (t, 1H, J=7.5 Hz), 7.43 (t, 1H, J=7.5 Hz), 7.59 (quint, 2H, J=7.8 Hz), 7.73 (d, 1H, J=8.4 Hz), 7.84 (d, 1H, J=8.4 Hz), 7.99 (d, 1H, J=7.5 Hz), 9.29 (d, 1H, J=7.5 Hz); MS m/z 1017 (2M+Na), 520 (M+Na).

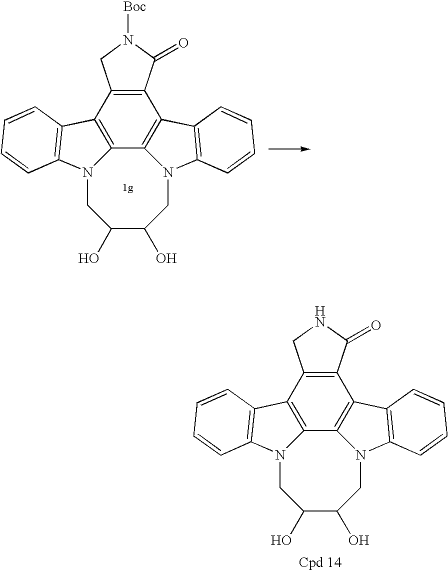

Compound 1g (158 mg) was dissolved in anhydrous TFA (10 mL) and the mixture was stirred for 2 hrs at r.t. The solvent was removed under vacuum and the resulting residue was diluted with ethyl ether, the solids were filtered and sequentially washed with DCM, MeOH and EtOAc (3 times each). The solvent was removed from the combined washings and then diluted with THF and filtered to provide Compound 14 (54 mg). The wash procedure was repeated to provide additional Compound 14 (31 mg) (combined yield 85 mg, 67%). ¹H NMR (300 MHz, d⁶-DMSO): δ 4.33 (bs, 2H), 4.62 (m, 1H), 4.64 (s, 2H), 4.79 (m, 1H), 4.99 (s, 2H), 5.47 (s, 2H), 7.31 (t, 1H, J=5 Hz), 7.40 (t, 1H, J=5 Hz), 7.55 (t, 1H, J=5 Hz), 7.6 (t, 1H, J=5 Hz), 7.75 (d, 1H, J=6 Hz), 7.84 (d, 1H, J=6 Hz), 8.08 (d, 1H, J=5 Hz), 8.62 (s, 1H), 9.45 (d, 1H, J=6 Hz); MS m/z/z 817 (2M+Na), 795 (2M+H), 398 (M+H); Anal. Calc. For C₂₄H₁₉N₃O₃+0.33C₂F₃HO₂: C, 68.04; H, 4.48; N, 9.65. Found: C, 68.11; H, 4.61; N, 9.70.

Using the procedure of Example 1, the following compounds were synthesized:

| Cpd | Name and Data |
|---|---|
| 2 | 12,13-(but-2-en-1,4-yl)-6,7,12,13-tetrahydro-5-oxo-5H-indolo[2,3-a]pyrrolo[3,4- |
| | c]carbazole |
| | 1H NMR(300MHz, d6-DMSO): δ 4.93(s, 2H), 5.39(d, 2H, J=5.4Hz), 5.44(d, |
| | 2H, J=5.4Hz), 6.51(bs, 2H), 7.27(t, 1H, J=7.2Hz), 7.37(t, 1H, J=7.5Hz), |
| | 7.55(quint, 2H, J=7.8Hz), 7.89(d, 1H, J=8.7Hz), 7.99(d, 1H, J=8.4Hz), 8.03(d, 1H, |
| | J=8.1Hz), 8.57(s, 1H), 9.59(d, 1H, J=7.8Hz); MS m/z 749(2M+Na), |
| | 727(2M+H), 364(M+H) |
| 4 | 12,13-(2,3-dimethyl-but-2-en-1,4-yl)-6,7,12,13-tetrahydro-5-oxo-5H-indolo[2,3- |
| | a]pyrrolo[3,4-c]carbazole |
| | ¹H NMR(DMSO) 1.73(s, 6H), 4.95(s, 2H), 5.36(s, 2H), 5.39(s, 2H), 7.28(t, 1H, |
| | J=8Hz), 7.38(t, 1H, J=8Hz), 7.56(quint, 2H, J=8Hz), 7.94(d, 1H, J=9Hz), |
| | 8.04(d, 2H, J=8Hz), 8.57(s, 1H), 9.55(d, 1H, J=8Hz); MS m/z 805(2M+Na), |
| | 783(2M+H), 392(M+H) |
| 5 | 12,13-(hex-3-en-1,6-yl)-6,7,12,13-tetrahydro-5-oxo-5H-indolo[2,3-a]pyrrolo[3,4- |
| | c]carbazole |
| | ¹H NMR(300MHz, d⁶-DMSO): δ 2.52(bs, 4H), 4.98(s, 2H), 5.14(m, 4H), |
| | 5.36(m, 2H), 7.29(t, 1H, J=7.5Hz), 7.39(t, 1H, J=7.5Hz), 7.56(quint, 2H, J=7.2Hz), |
| | 7.8(d, 1H, J=8.7Hz), 7.89(d, 1H, J=8.1Hz), 8.05(d, 1H, J=7.8Hz), 8.58(s, 1H), |
| | 9.6(d, 1H, J=7.8Hz); MS m/z 783(2M+H), 392(M+H) |
| 6 | 3-bromo-12,13-(but-2-en-1,4-yl)-6,7,12,13-tetrahydro-5-oxo-5H-indolo[2,3- |
| | a]pyrrolol [3,4-c]carbazole MS m/z 442(M+H) |
| 20 | 12,13-(2,3-dimethyl-2,3-dihydroxy-butan-1,4-yl)-6,7,12,13-tetrahydro-5-oxo-5H- |
| | indolo[2,3-a]pyrrolo[3,4-c]carbazole MS m/z 851(2M+Na), 426(M+H) |
| 21 | 12,13-(3,4-dihydroxy-hexan-1,6-yl)-6,7,12,13-tetrahydro-5-oxo-5H-indolol[2,3- |
| | a]pyrrolo[3,4-c]carbazole |
| | ¹H NMR(300MHz, d⁶-DMSO): δ 3-4.4(m, 12H), 4.91(s, 2H), 7.34(t, 1H, J=8Hz), |
| | 7.42(t, 1H, J=8Hz), 7.58(quint, 2H, J=7Hz), 7.71(d, 1H, J=8Hz), 7.78(d, |
| | 1H, J=8Hz), 8.06(d, 1H, J=8Hz), 8.59(s, 1H), 9.39(d, 1H, J=8Hz); MS m/z |
| | 851(2M+H), 426(M+H) |

### Example 2

3-[(4,5-dihydro-1H-imidazol-2-yl)hydrazomethylene]-12,13-(2,3-dihydroxy-butan-1,4-yl)-6,7,12,13-tetrahydro-5-oxo-5H-indolo[2,3-a]pyrrolo[3,4-c]carbazole (Compound 200)

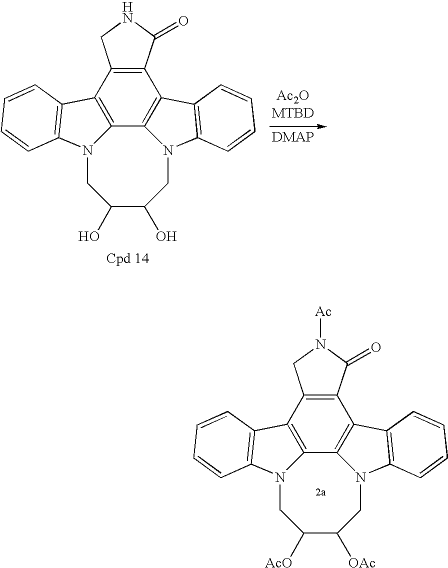

MTBD (0.9 mL) and acetic anhydride (5 mL) were added to a stirred solution of 12,13-(2,3-dihydroxy-butan-1,4-yl)-6,7,12,13-tetrahydro-5-oxo-5H-indolo[2,3-a]pyrrolo[3,4-c]carbazole Compound 12 (2.4 g, 6.04 mmoles) and N,N-dimethylamino-pyridine (3 g). The solution was stirred for 3 days at room temperature, then the solvent was removed under vacuum, the residue was diluted with water and the resulting solids filtered. The solids were washed with H₂O (4 times), and ethyl ether (4 times). The solids were dissolved in CHCl₃ and the solution was filtered over a plug of silica gel. The CHCl₃ layer was discarded, and the silica plug washed with ethyl acetate (800 mL). The ethyl acetate was removed and the residue was diluted with ethyl ether, then the resulting solids were filtered, washed with ether (4 times) and dried to provide 6-acetyl-12,13-(2,3-diacetoxy-butan-1,4-yl)-6,7,12,13-tetrahydro-5-oxo-5H-indolo[2,3-a]pyrrolo[3,4-c]carbazole Compound 2a (2.66 g, 84% yield) as a yellow solid. ¹H NMR (300 MHz, CDCl₃): δ 2.09 (s, 6H), 2.51 (s, 3H), 3.91 (bs, 2H), 4.18 (d, 1H, J=14 Hz), 4.37 (quint, 2H, J=15 Hz), 4.67 (d, 1H, J=16 Hz), 5.36 (m, 2H), 7.37 (m, 3H), 7.48 (d, 1H, J=8 Hz), 7.57 (t, 2H, J=8 Hz), 7.78 (d, 1H, J=8 Hz), 9.28 (d, 1H, J=8 Hz); MS d/z 1069 (2M+Na), 524 (M+Na).

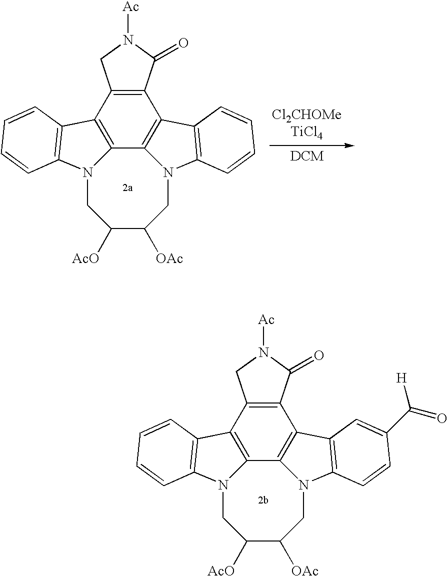

A 1 M solution of titanium tetrachloride (20 mL) in DCM was added to a solution of Compound 2a (1.01 g) in dichloromethoxymethane (4 mL) and DCM (220 mL). The reaction mixture was stirred overnight at room temperature. The reaction was quenched with a NaHCO₃ (aq.) solution and then extracted with CHCl₃ twice. The organic layers were washed with a NaCl (aq.) solution. The layers were combined and the solvent was removed under vacuum. The solids were precipitated by ether, filtered and washed with ether three times, then dissolved in CHCl₃ and filtered over a plug of silica gel. The CHCl₃ layer was discarded and the silica gel washed with ethyl acetate/methanol mixture (5:1). The solvent was removed and a solid was precipitated with ether. The solid was filtered, washed with ether and dried to provide 3-formyl-6-acetyl-12,13-(2,3-diacetoxy-1,4-butyl)-6,7,12,13-tetrahydro-5-oxo-5H-indolo[2,3-a]pyrrolo[3,4-c]carbazole Compound 2b (808 mg) as a light yellow solid. ¹H NMR (300 MHz, CDCl₃): δ 2.12 (s, 3H), 2.15 (s, 3H), 2.52 (s, 3H), 4.45 (m, 2H), 4.7 (m, 2H), 4.77 (m, 2H), 4.9 (d, 2H, J=18 Hz), 7.39 (d, 1H, J=8 Hz), 7.43 (t, 1H, J=8 Hz), 7.58 (d, 1H, J=8 Hz), 7.63 (t, 1H, J=8 Hz), 7.63 (t, 1H, J=8 Hz), 7.87 (d, 1H, J=8 Hz), 8.01 (d, 1H, J=8 Hz), 9.6 (s, 1H), 10.07 (s, 1H); MS m/z 552 (M+H).

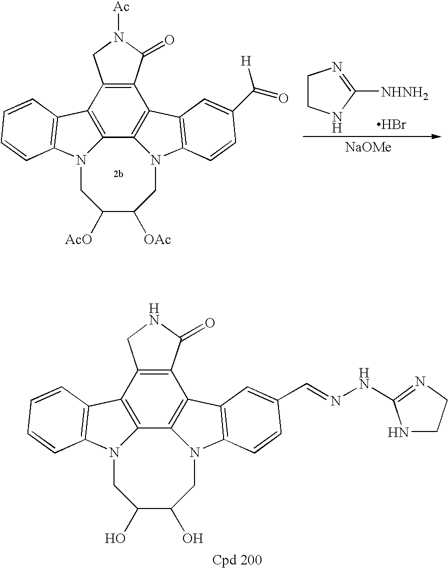

(4,5-dihydro-1H-imidazol-2-yl)-hydrazine (182 mg) was added to a solution of Compound 2b (141 mg) in methanol (10 mL). The solution was heated to 65° C. for 5 hours. The reaction mixture was cooled to room temperature and diluted with THF (10 mL). A 0.5 M solution of NaOMe/methanol (2 mL) was added and the mixture was stirred at room temperature overnight. The solids were filtered and washed with methanol and tetrahydrofuran (three times each). The solvent was removed from the washings. Water was added and the solids were filtered, washed with water and ether and dried, then purified by reverse phase HPLC to provide Compound 200 (56 mg) as an off-white solid. ¹H NMR (300 MHz, DMSO): δ 3.43 (quart, 4H, J=5 Hz), 4.33 (m, 2H), 4.63 (m, 2H), 4.56 (m, 2H), 4.75 (m, 2H), 4.99 (s, 2H), 5.48 (bs, 2H), 6.39 (s, 1H), 6.69 (s, 1H), 7.4 (t, 1H, J=8 Hz), 7.6 (t, 1H, J=8 Hz), 7.73 (d, 1H, J=9 Hz), 7.83 (d, 1H, J=8 Hz), 8.07 (d, 1H, J=8 Hz), 8.2 (s, 1H), 8.22 9 m, 1H), 8.59 (s, 1H), 9.42 (s, 1H); MS m/z 530 (M+Na), 508 (M+H).

Using the procedure of Example 2, the following compounds were synthesized:

| Cpd | Name and Data |
|---|---|
| 15 | 12,13-{2,3-bis-[(dimethylaminomethyl)carbonyloxy]-butan-1,4-yl}-6,7,12,13- |
| | tetrahydro-5-oxo-5H-indolo[2,3-a]pyrrolo[3,4-c]carbazole MS m/z 568(M+H) |
| 188 | 3-bromo-12,13-(2,3-dihydroxy-butan-1,4-yl)-6,7,12,13-tetrahydro-5-oxo-5H- |
| | indolo[2,3-a]pyrrolo[3,4-c]carbazole MS m/z 476(M+H) |
| 189 | 3-hydroxy-12,13-(2,3-dihydroxy-butan-1,4-yl)-6,7,12,13-tetrahydro-5-oxo-5H- |
| | indolo[2,3-a]pyrrolo[3,4-c]carbazole |
| | ¹H NMR(300MHz, DMSO): δ 4.31(m, 2H), 4.62(m, 2H), 4.71(m, 2H), |
| | 4.97(s, 2H), 7.05(dd, 1H, J=1Hz, J=9Hz), 7.39(t, 1H, J=7Hz), 7.55(d, 1H, J=9Hz), |
| | 7.6(d, 1H, J=7Hz), 7.82(d, 1H, J=8Hz), 8.07(d, 1H, J=8Hz), 8.55(s, 1H), |
| | 8.86(d, 1H, J=2Hz); MS m/z 877(2M+Na), 428(M+H) |
| 190 | 3-hydroxymethyl-12,13-(2,3-dihydroxy-butan-1,4-yl)-6,7,12,13-tetrahydro-5-oxo- |
| | 5H-indolo[2,3-a]pyrrolo[3,4-c]carbazole |
| | ¹H NMR(300MHz, DMSO): δ 4.31(m, 2H), 4.62(m, 4H), 4.71(m, 2H), |
| | 4.97(s, 2H), 7.38(t, 1H, J=7Hz), 7.52(dd, 1H, J=2Hz, J=9Hz), 7.58(t, 1H, J=8Hz), |
| | 7.68(d, 1H, J=9Hz), 7.82(d, 1H, J=8Hz), 8.06(d, 1H, J=8Hz), 8.56(s, 1H), |
| | 9.35(s, 1H); MS m/z 877(2M+Na), 428(M+H) |
| 191 | 3-nitro-12,13-(2,3-dihydroxy-butan-1,4-yl)-6,7,12,13-tetrahydro-5-oxo-5H- |
| | indolo[2,3-a]pyrrolo[3,4-c]carbazole |
| | ¹H NMR(300MHz, DMSO): δ 4.31(m, 2H), 4.72(m, 4H), 4.93(m, 2H), |
| | 5.48(m, 2H), 7.42(t, 1H, J=7Hz), 7.64(t, 2H, J=8Hz), 7.86(d, 1H, J=8Hz), 7.92(d, |
| | 1H, J=9Hz), 8.06(d, 1H, J=8Hz), 8.35(d, 1H, J=8Hz), 8.75(s, 1H), 10.33(s, |
| | 1H); MS m/z 443(M+H) |
| 192 | 3-amino-12,13-(2,3-dihydroxy-butan-1,4-yl)-6,7,12,13-tetrahydro-5-oxo-5H- |
| | indolo[2,3-a]pyrrolo[3,4-c]carbazole |
| | ¹H NMR(300MHz, DMSO): δ 4.31(d, 2H, J=7Hz), 4.66(m, 2H), 4.77(m, 2H), |
| | 5.02(s, 2H), 5.43(bs, 2H), 7.41(t, 1H, J=7Hz), 7.46(d, 2H, J=8Hz), 7.61(t, 1H, |
| | J=7Hz), 7.87(t, 1H, J=8Hz), 8.1(d, 1H, J=8Hz), 8.69(s, 1H), 9.46(s, 1H), |
| | 9.79(bs, 1H); MS m/z 825(2M+H), 413(M+H) |
| 193 | 3-(4-methyl-piperazin-1-ylmethyl)-12,13-(2,3-dihydroxy-butan-1,4-yl)-6,7,12,13- |
| | tetrahydro-5-oxo-5H-indolo[2,3-a]pyrrolo[3,4-c]carbazole |
| | ¹H NMR(300MHz, DMSO): δ 2.17(s, 3H), 2.36(m, 8H), 3.64(s, 2H), 4.32(s, |
| | 2H), 4.62(m, 2H), 4.72(m, 2H), 4.98(s, 2H), 5.44(s, 2H), 7.39(t, 1H, J=7Hz), |
| | 7.49(d, 1H, J=8Hz), 7.59(t, 1H, J=7Hz), 7.68(d, 1H, J=8Hz), 7.83(d, 1H, |
| | J=8Hz), 8.07(d, 1H, J=7Hz), 8.56(s, 1H), 9.32(s, 1H); MS m/z 1019(2M+H), |
| | 510(M+H) |
| 194 | 3-(morpholin-4-ylmethyl)-12,13-(2,3-dihydroxy-butan-1,4-yl)-6,7,12,13- |
| | tetrahydro-5-oxo-5H-indolo[2,3-a]pyrrolo[3,4-c]carbazole |
| | ¹H NMR(300MHz, DMSO): δ 3.42(m, 8H), 3.64(t, 2H, J=12Hz), 3.96(d, 2H, |
| | J=12Hz), 4.34(m, 2H), 4.58(m, 2H), 4.71(m, 2H), 5.01(s, 2H), 5.48(bs, 2H), |
| | 7.41(t, 1H, J=8Hz), 7.61(t, 1H, J=8Hz), 7.67(d, 1H, J=9Hz), 7.85(d, 1H, |
| | J=9Hz), 7.89(d, 1H, J=8Hz), 8.1(d, 1H, J=8Hz), 8.64(s, 1H), 9.56(s, 1H), |
| | 9.95(s, 1H); MS m/z 993(2M+H), 497(M+H) |
| 195 | 3-[(1-methyl-piperidin-4-yl)aminomethyl]-12,13-(2,3-dihydroxy-butan-1,4-yl)- |
| | 6,7,12,13-tetrahydro-5-oxo-5H-indolo[2,3-a]pyrrolo[3,4-c]carbazole |
| | ¹H NMR(300MHz, DMSO): δ 1.89(m, 2H), 2.41(m, 2H), 2.79(s, 3H), |
| | 3.04(bs, 2H), 3.65(m, 3H), 4.33(m, 2H), 4.42(bs, 2H), 4.68(m, 2H), 4.74(m, 2H), |
| | 5.01(s, 2H), 7.41(t, 1H, J=7Hz), 7.61(t, 1H, J=8Hz), 7.69(d, 1H, J=8Hz), |
| | 7.87(t, 1H, J=8Hz), 7.68(d, 1H, J=8Hz), 8.09(d, 1H, J=8Hz), 8.65(s, 1H), 9.21(bs, |
| | 1H), 9.54(s, 1H), 10.02(bs, 1H); MS m/z 1047(2M+H), 524(M+H) |
| 196 | 3-{[N-methyl-N-(1-methyl-piperidin-4-yl)]aminomethyl}-12,13-(2,3-dihydroxy- |
| | butan-1,4-yl)-6,7,12,13-tetrahydro-5-oxo-5H-indolo[2,3-a]pyrrolo[3,4- |
| | c]carbazole |
| | ¹H NMR(300MHz, DMSO): δ 2.08(m, 2H), 2.42(m, 2H), 2.75(s, 3H), 2.8(s, |
| | 3H), 3.12(bs, 2H), 3.63(m, 3H), 4.31(s, 2H), 4.34(s, 2H), 4.68(m, 2H), |
| | 4.74(m, 2H), 5.02(s, 2H), 7.42(t, 1H, J=7Hz), 7.62(t, 1H, J=8Hz), 7.71(d, 1H, |
| | J=9Hz), 7.87(d, 1H, J=8Hz), 7.91(d, 1H, J=9Hz), 8.11(d, 1H, J=8Hz), 8.65(s, |
| | 1H), 9.59(s, 1H); MS m/z 538(M+H) |
| 197 | 3-{[3-(4-methyl-piperazin-1-yl)prop-1-yl]aminomethyl}-12,13-(2,3-dihydroxy- |
| | butan-1,4-yl)-6,7,12,13-tetrahydro-5-oxo-5H-indolo[2,3-a]pyrrolo[3,4- |
| | c]carbazole |
| | ¹H NMR(300MHz, DMSO): δ 1.89(m, 2H), 2.59(m, 2H), 2.77(s, 3H), |
| | 3.05(bs, 2H), 3.65(m, 8H), 4.33(m, 2H), 4.33(m, 2H), 4.37(m, 2H), 4.67(m, 2H), |
| | 4.74(m, 2H), 5.02(s, 2H), 7.41(t, 1H, J=8Hz), 7.62(t, 1H, J=8Hz), 7.68(dd, |
| | 1H, J=2Hz, J=8Hz), 7.87(t, 1H, J=8Hz), 8.1(d, 1H, J=8Hz), 8.65(s, 1H), |
| | 8.94(bs, 1H), 9.52(s, 1H); MS m/z 567(M+H) |
| 198 | 3-[(2-methoxy-ethyl)aminomethyl]-12,13-(2,3-dihydroxy-butan-1,4-yl)-6,7,12,13- |
| | tetrahydro-5-oxo-5H-indolo[2,3-a]pyrrolo[3,4-c]carbazole |
| | ¹H NMR(300MHz, DMSO): δ 3.18(bs, 2H), 3.34(s, 3H), 3.39(bs, 2H), 3.63(t, |
| | 2H, J=5Hz), 4.34(m, 2H), 4.67(m, 2H), 4.73(m, 2H), 5.0(s, 2H), 5.45(bs, 2H), |
| | 7.4(t, 1H, J=8Hz), 7.61(t, 1H, J=8Hz), 7.68(d, 1H, J=8Hz), 7.85(d, 2H, |
| | J=8Hz), 8.09(d, 1H, J=8Hz), 8.63(s, 1H), 8.69(bs, 1H), 9.52(s, 1H); MS m/z |
| | 991(2M+Na), 485(M+H) |
| 199 | 3-[(2-dimethylamino-ethyl)thiomethyl]-12,13-(2,3-dihydroxy-butan-1,4-yl)- |
| | 6,7,12,13-tetrahydro-5-oxo-5H-indolo[2,3-a]pyrrolo[3,4-c]carbazole |
| | ¹H NMR(300MHz, DMSO): δ 2.82(s, 6H), 4.06(s, 2H), 4.3(bs, 2H), 4.61(m, |
| | 2H), 4.75(m, 2H), 4.99(s, 2H), 5.46(bs, 2H), 7.39(t, 1H, J=8Hz), 7.53(d, 1H, |
| | J=8Hz), 7.6(t, 1H, J=8Hz), 7.73(d, 1H, J=8Hz), 7.83(d, 1H, J=8Hz), 8.08(d, |
| | 1H, J=8Hz), 8.6(s, 1H), 9.38(s, 1H), 9.48(bs, 1H); MS m/z 1051(2M+Na), |
| | 515(M+H) |
| 201 | 3-(1-imidazolin-1-yl-prop-2-en-3-yl)-12,13-(2,3-dihydroxy-butan-1,4-yl)- |
| | 6,7,12,13-tetrahydro-5-oxo-5H-indolo[2,3-a]pyrrolo[3,4-c]carbazole |
| | ¹H NMR(300MHz, DMSO): δ 4.3(d, 2H, J=7Hz), 4.63(m, 2H), 4.71(m, 2H), |
| | 4.99(m, 2H), 5.08(d, 2H, J=7Hz), 5.45(bs, 2H), 6.47(m, 1H), 6.98(d, 1H, |
| | J=6Hz), 7.39(m, 1H), 7.59(t, 1H, J=8Hz), 7.73(m, 2H), 7.83(d, 1H, J=8Hz), |
| | 7.86(s, 1H), 8.07(d, 1H, J=8Hz), 8.6(s, 1H), 9.21(s, 1H), 9.52(s, 1H); MS m/z |
| | 1007(2M+H), 504(M+H) |
| 202 | 3-(imidazol-1-ylmethyl)-12,13-(2,3-dihydroxy-butan-1,4-yl)-6,7,12,13-tetrahydro- |
| | 5-oxo-5H-indolo[2,3-a]pyrrolo[3,4-c]carbazole |
| | ¹H NMR(300MHz, DMSO): δ 4.28(m, 2H), 4.59(m, 2H), 4.75(m, 2H), |
| | 4.99(s, 2H), 5.44(bs, 1H), 5.65(s, 2H), 7.39(t, 1H, J=8Hz), 7.59(m, 2H), 7.71(d, 1H, |
| | J=2Hz), 7.78(s, 1H), 7.79(d, 1H, J=5Hz), 7.84(d, 1H, J=5Hz), 8.08(d, 1H, |
| | J=8Hz), 8.59(s, 1H), 9.26(s, 1H), 9.43(s, 1H); MS m/z 977(2M+Na), |
| | 478(M+H) |
| 203 | 3-(2-morpholin-4-yl-ethoxy)-12,13-(2,3-dihydroxy-butan-1,4-yl)-6,7,12,13- |
| | tetrahydro-5-oxo-5H-indolo[2,3-a]pyrrolo[3,4-c]carbazole |
| | ¹H NMR(300MHz, DMSO): δ 3.53(m, 4H), 3.63(m, 2H), 3.77(m, 2H), |
| | 4.0(m, 2H), 4.29(d, 2H, J=6Hz), 4.47(m, 2H), 4.6(m, 2H), 4.66(m, 2H), 4.98(s, |
| | 2H), 5.42(bs, 1H), 7.28(dd, 1H, J=3Hz, J=9Hz), 7.38(t, 1H, J=8Hz), 7.59(t, |
| | 1H, J=8Hz), 7.7(d, 1H, J=9Hz), 7.82(d, 1H, J=8Hz), 8.06(d, 1H, J=8Hz), |
| | 8.56(s, 1H), 9.15(d, 1H, J=3Hz), 10.15(bs, 1H); MS m/z 1075(2M+Na), 527(M+H) |
| 204 | 3-[(1-methyl-piperidin-4-yl)amino]-12,13-(2,3-dihydroxy-butan-1,4-yl)-6,7,12,13- |
| | tetrahydro-5-oxo-5H-indolo[2,3-a]pyrrolo[3,4-c]carbazole |
| | ¹H NMR(300MHz, DMSO): δ 1.77(m, 2H), 2.084(m, 1H), 2.25(d, 2H, |
| | J=14Hz), 2.81(s, 3H), 3.35(m, 4H), 4.29(d, 2H, J=7Hz), 4.63(m, 2H), 4.75(m, |
| | 2H), 4.98(s, 2H), 7.27(bs, 1H), 7.39(t, 1H, J=7Hz), 7.59(t, 1H, J=8Hz), |
| | 7.74(bs, 1H), 7.83(d, 1H, J=8Hz), 8.07(d, 1H, J=8Hz), 8.56(s, 1H), 9.51(bs, 1H); |
| | MS m/z 1041(2M+H), 509(M+H) |
| 205 | 3-{[(4-methyl-piperazin-1-yl)methylcarbonyl]amino}-12,13-(2,3-dihydroxy- |
| | butan-1,4-yl)-6,7,12,13-tetrahydro-5-oxo-5H-indolo[2,3-a]pyrrolo[3,4- |
| | c]carbazole |
| | ¹H NMR(300MHz, DMSO): δ 2.81(s, 3H), 3.18(m, 10H), 4.3(d, 2H, J=7Hz), |
| | 4.63(m, 2H), 4.75(m, 2H), 4.98(s, 2H), 7.39(t, 1H, J=8Hz), 7.59(t, 1H, |
| | J=7Hz), 7.71(d, 1H, J=9Hz), 7.83(d, 1H, J=8Hz), 7.89(d, 1H, J=9Hz), 8.07(d, |
| | 1H, J=8Hz), 8.55(s, 1H), 9.35(s, 1H), 9.99(s, 1H); MS m/z 1127(2M+Na), |
| | 553(M+H) |
| 206 | 3-[4,5-dihydro-imidazol-2-yl)amino]-12,13-(2,3-dihydroxy-butan-1,4-yl)- |
| | 6,7,12,13-tetrahydro-5-oxo-5H-indolo[2,3-a]pyrrolo[3,4-c]carbazole |
| | ¹H NMR(300MHz, DMSO): δ 3.67(s, 4H), 4.3(d, 2H, J=6Hz), 4.66(m, 2H), |
| | 4.77(m, 2H), 5(s, 2H), 5.46(bs, 1H), 7.4(t, 1H, J=7Hz), 7.44(d, 1H, J=11Hz), |
| | 7.61(t, 1H, J=7Hz), 7.87(d, 1H, J=9Hz), 8.09(d, 1H, J=8Hz), 8.16(s, 1H), |
| | 8.66(s, 1H), 9.34(d, 1H, J=2Hz), 10.44(s, 1H); MS m/z 961(2M+H), 481(M+H) |

### Example 3

12,13-(2-methylene-propan-1,3-yl)-6,7,12,13-tetrahydro-5-oxo-5H-indolo[2,3-a]pyrrolo[3,4-c]carbazole (Compound 22)
12,13-(2-hydroxymethyl-propan-1,3-yl)-6,7,12,13-tetrahydro-5-oxo-5H-indolo[2,3-a]pyrrolo[3,4-c]carbazole (Compound 23)
9-hydroxymethyl-12,13-(2-hydroxymethyl-propan-1,3-yl)-6,7,12,13-tetrahydro-5-oxo-5H-indolo[2,3-a]pyrrolo[3,4-c]carbazole (Compound 212)

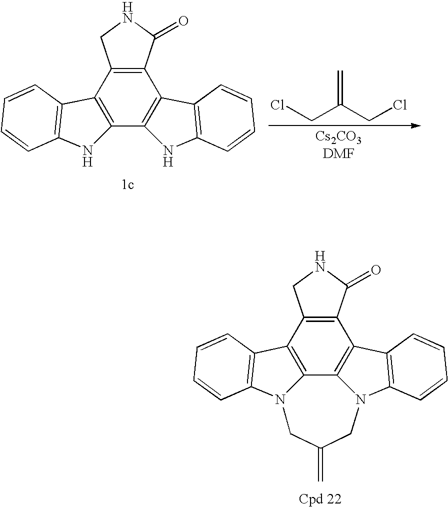

Compound 1c (1.56 g, 5.0 mmol, 1.0 equiv) was added to Cs₂CO₃ (4.89 g, 15.0 mmol, 3.0 equiv) in DMF (20 mL) under N₂ at room temperature. The mixture was stirred for 5 min, then 3-chloro-2-chloromethyl-prop-1-ene (625 mg, 5.0 mmol, 1.0 equiv) was added. The mixture was stirred at rt for 24 hours and poured into H₂O (200 mL). The resulting yellow solid was filtered and washed with H₂O (50 mL×2) and Et₂O/hexane (1:1, 50 mL×3), then dried in vacuo to provide Compound 22 (1.46 g, 80%) as a yellow solid. ¹H NMR: (d⁶-DMSO) δ 4.96 (s, 2H), 5.28 (s, 2H), 5.30 (s, 2H), 5.80 (s, 2H), 7.28 (t, 1H, J=8.1 Hz), 7.37 (t, 1H, J=7.2 Hz), 7.52 (t, 1H, J=8.1 Hz), 7.57 (t, 1H, J=8.1 Hz), 7.81 (d, 1H, J=8.1 Hz), 7.87 (d, 1H, J=7.8 Hz), 8.07 (d, 1H, J=7.2 Hz), 8.53 (s, 1H), 9.31 (d, 1H, J=7.8 Hz). MS m/z 386 (M+Na), 364 (M+H).

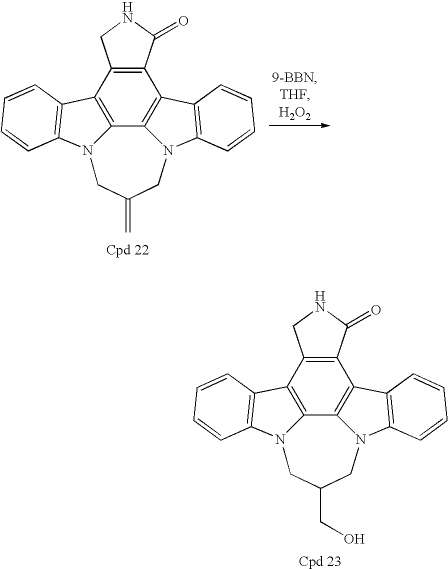

9-BBN (0.5 M in THF, 20 mL, 10 mmol) was added to a solution of Compound 22 (363 mg, 1.0 mmol) in THF (20 mL) under N₂ at room temperature. The mixture was stirred at 60-65° C. for 3 hours, then cooled to 0° C. MeOH (5 mL) was added dropwise and the mixture was stirred for 10 min. H₂O₂ (50% wt in H₂O, 5 μL) was added dropwise and the mixture was stirred for 10 min. 10% NaOH(aq) was added and the mixture was heated to 65° C. for 1 hour. The solvent was removed and the resulting solid was filtered and washed with H₂O (20 mL) and Et₂O/hexane (1/1, 30 mL×3), then dried in vacuo to provide Compound 23 (309 mg, 81%) as a yellow solid. ¹H NMR: (d⁶-DMSO) δ 2.82 (m, 1H), 3.70 (t, 2H, J=5.4 Hz), 4.53-4.59 (m, 2H), 4.83-4.88 (m, 2H), 4.97 (s, 2H), 5.08 (t, 1H, J=5.4 Hz), 7.28 (t, 1H, J=7.5 Hz), 7.37 (t, 1H, J=7.8 Hz), 7.51 (t, 1H, J=8.1 Hz), 7.56 (t, 1H, J=8.1 Hz), 7.72 (d, 1H, J=8.7 Hz), 7.79 (d, 1H, J=8.4 Hz), 8.08 (d, 1H, J=7.5 Hz), 8.53 (s, 1H), 9.32 (d, 1H, J=7.8 Hz). MS m/z 404 (M+Na), 382 (M+H).

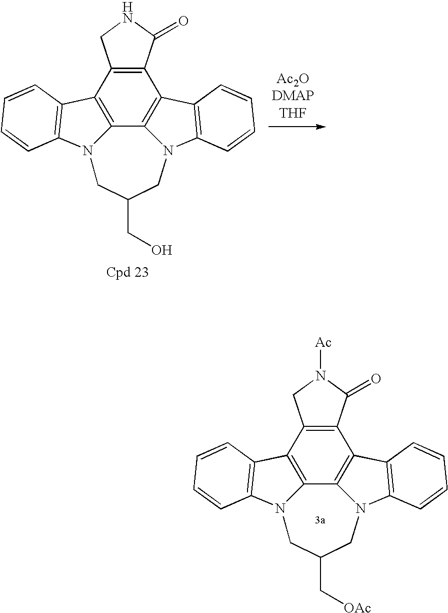

Ac₂O (2.04 g, 20 mmol) was added to a suspension of Compound 23 (762 mg, 2.0 mmol) and DMAP (1.22 g, 10 mmol) in THF (50 mL) at room temperature. The mixture was stirred at room temperature for 2 hours, then heated to 50° C. for 12 hours. The mixture was concentrated and the residue was poured into H₂O (20 mL). The resulting solid was filtered and washed with H₂O (20 mL) and Et₂O/hexane (1/1, 50 mL) to provide Compound 3a (765 mg, 82%) as a yellow solid, which was used in the next step without further purification.

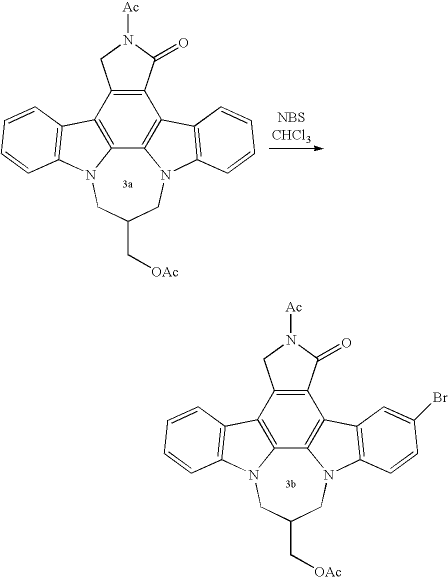

NBS (534 mg, 3.0 mmol) was added to a solution of Compound 3a (1.395 g, 3.0 mmol) in CHCl₃/MeOH (50 mUS50 mL) at room temperature under N₂. The mixture was stirred at rt for 1 hour, then poured into EtOAc/H₂O (200 mL/150 mL). The organic layer washed with H₂O (150 mL) and saturated NaHCO₃(aq) (150 mL), then dried over Na₂SO₄(s) and filtered. The solvent was removed and the product was resolidified from acetone/hexane to give Compound 3b (1.32 g, 81%) as yellow solid, which was used in the next step without further purification.

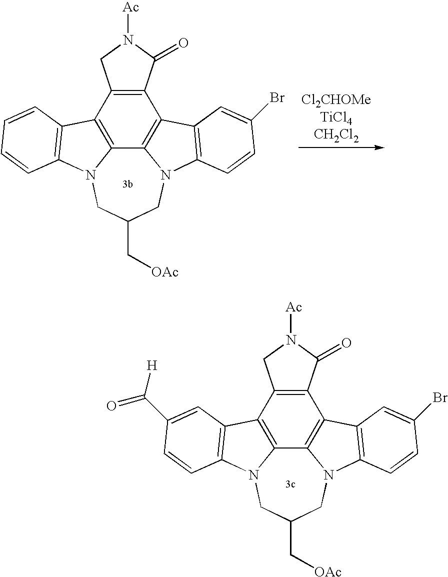

Dichloromethyl methyl ether (3.45 g, 30 mmol) and TiCl₄ (1.0 M in CH₂Cl₂, 15 mL, 15.0 mmol) were added to a solution of Compound 3b (814 mg, 1.5 mmol) in CH₂Cl₂ (60 mL) at room temperature under N₂. The mixture was stirred at room temperature for 24 hours and was poured into CH₂Cl₂/H₂O (100 mL/200 mL). The aqueous layer was extracted with CH₂Cl₂ (100 mL). The combined organic layer was washed with NaCl (aq.) (200 mL), dried over Na₂SO₄ and filtered. The solvent was removed and the product was resolidified from acetone/hexane to give Compound 3c (711 mg, 83%) as a yellow solid, which was used in the next step without further purification.

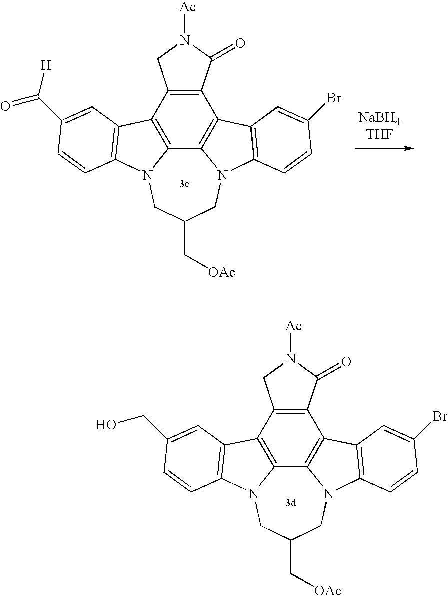

NaBH₄ (160 mg, 5.0 mmol) was added in one portion to a solution of Compound 3c (571 mg, 1.0 mmol) in THF (30 mL) under N₂ at room temperature. The mixture was stirred at room temperature for 1 hour and was poured into a mixture of EtOAc/saturated NH₄Cl (aq) (150 mL/100 mL). The organic layer washed with brine (150 mL), dried over Na₂SO₄ and filtered. The solvent was removed and the product was resolidified from acetone/hexane to give Compound 3d (413 mg, 72%) as a yellow solid, which was used in the next step without further purification.

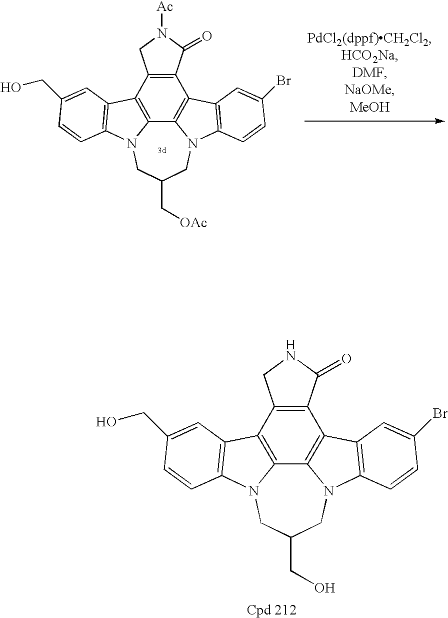

[PdCl₂(dppf).CH₂Cl₂] (4 mg) was added to a mixture of Compound 3d (29 mg, 0.05 mmol) and sodium formate (34 mg, 0.5 mmol) in DMF (2 mL) at room temperature under N₂. The mixture was heated to 100° C. for 1.5 hours, then poured into EtOAc/hexane (100 mL/100 mL). The aqueous layer was extracted with EtOAc (100 mL×2) and the combined organic layers were dried over Na₂SO₄ and filtered. The solvent was removed and the crude product was dissolved in THF (5 mL) and NaOMe (0.5 M in MeOH, 5 mL) was added. The mixture was stirred at room temperature for 1 hour, then was poured into a mixture of EtOAc/H₂O (100 mL/100 mL) and the aqueous layer was extracted with EtOAc (100 mL×2). The combined organic layers were dried over Na₂SO₄ and filtered. The solvent was removed and the crude product was purified by silica gel chromatography using MeOH/acetone/hexane (May 25, 1970 to May 45, 1950) to give Compound 212 (12 mg, 58%) as a pale yellow solid. ¹H NMR: (d⁶-DMSO) δ 2.82 (t, 1H, J=6.9 Hz), 3.68 (t, 2H, J=5.4 Hz), 4.48-4.57 (m, 2H), 4.73 (d, 2H, J=5.7 Hz), 4.85 (d, 2H, J=13.2 Hz), 4.95 (s, 2H), 5.08 (t, 1H, J=5.1 Hz), 5.23 (t, 1H, J=6.0 Hz), 7.28 (t, 1H, J=7.5 Hz), 7.54-7.48 (m, 2H), 7.72 (d, 1H, J=8.1 Hz), 7.74 (d, 1H, J=8.4 Hz), 8.00 (s, 1H), 8.51 (s, 1H), 9.32 (d, 1H, J=7.8 Hz). MS m/z 434 (M+Na), 412 (M+H).

Using the procedure of Example 3, the following compounds were synthesized:

| Cpd | Name and Data |
|---|---|
| 25 | 12,13-(2-hydroxy-2-hydroxymethyl-propan-1,3-yl)-6,7,12,13-tetrahydro-5-oxo-5H- |
| | indolo[2,3-a]pyrrolo[3,4-c]carbazole |
| | ¹H NMR: (d⁶-DMSO) δ 3.72(d, 2H, J=5.2Hz), 4.59-4.53(m, 2H), 4.82-4.79(m, 2H), |
| | 4.97(s, 2H), 5.29(t, 1H, J=5.6Hz), 5.39(s, 1H), 7.28(t, 1H, J=7.6Hz), |
| | 7.32(t, 1H, J=7.6Hz), 7.51(t, 1H, J=8.0Hz), 7.56(t, 1H, J=7.2Hz), 7.67(d, 1H, |
| | J=8.0Hz), 7.75(d, 1H, J=8.0Hz), 8.09(d, 1H, J=7.6Hz), 8.52(s, 1H), |
| | 9.32(d, 1H, J=8.0Hz). MS m/z 398(M+H) |
| 207 | 3-bromo-9-formyl-12,13-(2-hydroxymethyl-propan-1,3-yl)-6,7,12,13-tetrahydro-5- |
| | oxo-5H-indolo[2,3-a]pyrrolo[3,4-c]carbazole |
| | ¹H NMR: (d⁶-DMSO) δ 2.83(t, 1H, J=5.1Hz), 3.77(d, 2H, J=6.0Hz), |
| | 4.47-4.96(m, 4H), 4.98(s, 2H), 5.01(br s, 1H), 7.62(d, 1H, J=8.4Hz), 7.70(d, 1H, J=9.0Hz), |
| | 7.96(d, 1H, J=8.7Hz), 8.08(d, 1H, J=8.4Hz), 8.60(s, 1H), 8.71(s, 1H), |
| | 9.48(d, 1H, J=1.5Hz), 10.16(s, 1H) |
| 208 | 3-bromo-9-(morpholin-4-ylmethyl)-12,13-(2-hydroxymethyl-propan-1,3-yl)- |
| | 6,7,12,13-tetrahydro-5-oxo-5H-indolo[2,3-a]pyrrolo[3,4-c]carbazole |
| | ¹H NMR: (d⁶-DMSO) δ 2.83(s, 1H), 3.14-3.41(m, 4H), 3.59-3.66(m, 4H), 3.98(d, |
| | 2H, J=10.8Hz), 4.52-4.62(m, 2H), 4.86(t, 2H, J=8.4Hz), 4.99(s, 2H), |
| | 7.62-7.69(s, 2H), 7.74(d, 1H, J=8.7Hz), 7.91(d, 1H, J=8.7Hz), 8.25(s, 1H), |
| | 8.70(s, 1H), 9.49(d, 1H, J=2.1Hz), 10.00-10.10(br s, 1H) |
| 209 | 3-bromo-9-(isopropoxymethyl)-12,13-(2-hydroxymethyl-propan-1,3-yl)-6,7,12,13- |
| | tetrahydro-5-oxo-5H-indolo[2,3-a]pyrrolo[3,4-c]carbazole |
| | ¹H NMR: (d⁶-DMSO) δ 1.20(d, 3H, J=6.3Hz), 1.23(d, 3H, J=6.9Hz), |
| | 2.60-2.70(m, 1H), 3.66-3.76(m, 3H), 4.54-4.58(m, 2H), 4.69(s, 2H), 4.82-4.88(m, 2H), |
| | 4.97(s, 2H), 7.56(d, 1H, J=8.4Hz), 7.63(d, 1H, J=8.7Hz), 7.72(d, 1H, J=8.7Hz), |
| | 7.77(d, 1H, J=8.7Hz), 8.00(s, 1H), 8.60(s, 1H), 9.50(d, 1H, J=1.8Hz) |
| 210 | 3,9-bis-(isopropoxymethyl)-12,13-(2-hydroxymethyl-propan-1,3-yl)-6,7,12,13- |
| | tetrahydro-5-oxo-5H-indolo[2,3-a]pyrrolo[3,4-c]carbazole |
| | ¹H NMR: (d⁶-DMSO) δ 1.20(m, 12H), 2.75(m, 1H), 3.65-3.75(m, 4H), |
| | 4.51-4.54(m, 2H), 4.64(s, 2H), 4.68(s, 2H), 4.82-4.86(m, 2H), 4.95(s, 2H), 5.07(t, 1H, J=5.4Hz), |
| | 7.48(d, 1H, J=9.0Hz), 7.54(d, 1H, J=8.7Hz), 7.68(d, 1H, J=9.0Hz), |
| | 7.75(d, 1H, J=8.4Hz), 7.98(s, 1H), 8.48(s, 1H), 9.26(s, 1H). MS m/z |
| | 526(M+H) |
| 211 | 9-(isopropoxymethyl)-12,13-(2-hydroxymethyl-propan-1,3-yl)-6,7,12,13-tetrahydro- |
| | 5-oxo-5H-indolo[2,3-a]pyrrolo[3,4-c]carbazole |
| | ¹H NMR: (d⁶-DMSO) δ 1.20(d, 3H, J=6.0Hz), 1.22(d, 3H, J=5.1Hz), 2.79(m, 1H), |
| | 3.66-3.77(m, 3H), 4.43-4.56(m, 2H), 4.90(s, 2H), 4.79-4.85(m, 2H), 4.90(s, |
| | 2H), 5.07(t, 1H, J=5.1Hz), 7.28(t, 1H, J=7.5Hz), 7.48-7.58(m, 2H), |
| | 7.67-7.79(m, 2H), 7.95(s, 1H), 8.49(s, 1H), 9.32(d, 1H, J=8.1Hz). MS m/z 454(M+H) |
| 213 | 3-(isopropylaminomethyl)-12,13-(2-hydroxymethyl-propan-1,3-yl)-6,7,12,13- |
| | tetrahydro-5-oxo-5H-indolo[2,3-a]pyrrolo[3,4-c]carbazole MS m/z 453(M+H) |
| 214 | 3,9-bis-(morpholin-4-ylmethyl)-12,13-(2-hydroxymethyl-propan-1,3-yl)-6,7,12,13- |
| | tetrahydro-5-oxo-5H-indolo[2,3-a]pyrrolo[3,4-c]carbazole |
| | ¹H NMR: (d⁶-DMSO) δ 2.83(m, 1H), 3.00-3.40(br s, 4H), 3.67(d, 2H, J=6.3Hz), |
| | 3.87-4.00(br s, 4H), 4.53-4.62(m, 6H), 4.87(d, 2H, J=12.3Hz), 5.00(s, 2H), |
| | 5.17(br s, 1H), 7.72(d, 1H, J=8.4Hz), 7.74(d, 1H, J=8.4Hz), 7.83(d, 1H, J=8.4Hz), |
| | 7.86(d, 1H, J=8.7Hz), 8.44(s, 1H), 8.64(s, 1H), 9.40(s, 1H). MS: |
| | 602(M+Na), 580(M+H) |
| 215 | 3,9-bis-(hydroxymethyl)-12,13-(2-hydroxymethyl-propan-1,3-yl)-6,7,12,13- |
| | tetrahydro-5-oxo-5H-indolo[2,3-a]pyrrolo[3,4-c]carbazole |
| | ¹H NMR: (d⁶-DMSO) δ 2.81(br s, 1H), 3.67(t, 2H, J=6.0Hz), 4.48-4.54(m, 2H), |
| | 4.68(d, 1H, J=5.4Hz), 4.73(d, 1H, J=5.4Hz), 4.85(d, 2H, J=12.0Hz), 4.94(s, |
| | 2H), 5.12(t, 1H, J=4.8Hz), 5.17(t, 1H, J=6.0Hz), 5.27(t, 1H, J=5.7Hz), |
| | 7.50(d, 1H, J=8.1Hz), 7.53(d, 1H, J=8.1Hz), 7.67(d, 1H, J=8.7Hz), 7.74(d, 1H, |
| | J=8.4Hz), 8.00(s, 1H), 8.50(s, 1H), 9.24(s, 1H). MS: 464(M+Na), 442(M+H) |
| 216 | 3-[(2E)-2-pyridin-2-yl-ethenyl]-12,13-(2-hydroxymethyl-propan-1,3-yl)-6,7,12,13- |
| | tetrahydro-5-oxo-5H-indolo[2,3-a]pyrrolo[3,4-c]carbazole |
| | ¹H NMR: (d⁶-DMSO) δ 2.81-2.85(m, 1H), 3.69(br s, 2H), 4.55(br s, 2H). 4.86(d, |
| | 2H, J=12.9Hz), 4.96(s, 2H), 5.10(s, 1H), 7.25(t, 1H, J=7.5Hz), 7.29(d, 1H, J=13.2Hz), |
| | 7.38(t, 1H, J=7.5Hz), 7.55-7.62(m, 2H), 7.74-7.93(m, 5H), 8.07(d, |
| | 1H, J=7.8Hz), 9.58(s, 1H), 9.59(d, 1H, J=3.9Hz), 9.55(s, 1H). MS m/z |
| | 507(M+Na), 485(M+H) |
| 217 | 3-[(2E)-2-(4-methyl-thiazol-5-yl)ethenyl]-12,13-(2-hydroxymethyl-propan-1,3-yl)- |
| | 6,7,12,13-tetrahydro-5-oxo-5H-indolo[2,3-a]pyrrolo[3,4-c]carbazole MS m/z |
| | 505(M+H) |
| 218 | 3-[(2E)-2-carboxy-ethenyl]-12,13-(2-hydroxymethyl-propan-1,3-yl)-6,7,12,13- |
| | tetrahydro-5-oxo-5H-indolo[2,3-a]pyrrolo[3,4-c]carbazole MS m/z 452(M+H) |
| 219 | 3-[(1E)-3-1H-imidazol-1-yl-prop-1-en-1-yl]-12,13-(2-hydroxymethyl-propan-1,3-yl)- |
| | 6,7,12,13-tetrahydro-5-oxo-5H-indolo[2,3-a]pyrrolo[3,4-c]carbazole MS m/z |
| | 488(M+H) |
| 220 | 3-[(2E)-2-1H-imidazol-1-yl-ethenyl]-12,13-(2-hydroxymethyl-propan-1,3-yl)- |
| | 6,7,12,13-tetrahydro-5-oxo-5H-indolo[2,3-a]pyrrolo[3,4-c]carbazole MS m/z |
| | 474(M+H) |
| 221 | 3-{(1E)-3-[(4,5-dihydro-1H-imidazol-2-yl)amino]-prop-1-en-1-yl}-12,13-(2- |
| | hydroxymethyl-propan-1,3-yl)-6,7,12,13-tetrahydro-5-oxo-5H-indolo[2,3- |
| | a]pyrrolo[3,4-c]carbazole MS m/z 536(M+H) |
| 222 | 3-{(2E)-2-[(2-dimethylamino-ethyl)carbamoyl]ethenyl}-12,13-(2-hydroxymethyl- |
| | propan-1,3-yl)-6,7,12,13-tetrahydro-5-oxo-5H-indolo[2,3-a]pyrrolo[3,4-c]carbazole |
| | MS m/z 536(M+H) |
| 223 | 3-[(1E)-3-1H-imidazol-1-yl-prop-1-en-1-yl]-9-hydroxymethyl-12,13-(2- |
| | hydroxymethyl-propan-1,3-yl)-6,7,12,13-tetrahydro-5-oxo-5H-indolo[2,3- |
| | a]pyrrolo[3,4-c]carbazole MS m/z 517(M+H) |

### Example 4

12,13-(2,3-dihydroxy-butan-1,4-yl)-12,13-dihydro-5,7-dioxo-6H-indolo[2,3-a]pyrrolo[3,4-c]carbazole (Compound 9)

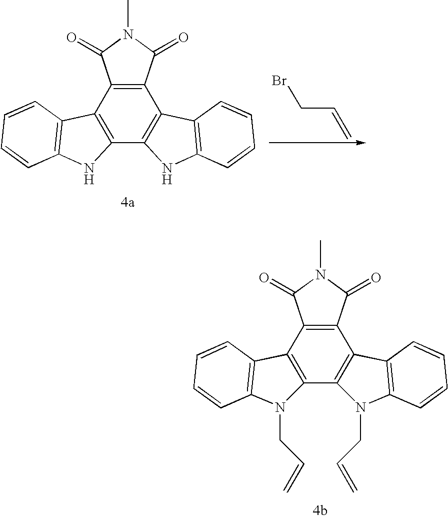

6-methyl-12,13-dihydro-5,7-dioxo-6H-indolo[2,3-a]pyrrolo[3,4-c]carbazole Compound 4a (2.37 g, 7 mmol) (prepared as described in Slater M J, Bioorganic & Medicinal Chemistry, 1999, 7, 1067) was dissolved in DMF (200 mL) and Cs₂CO₃ (5 g) was added, followed by 3-bromo-prop-1-ene (1.8 mL). The mixture was stirred at 20° C. for 24 hrs, then diluted with water (500 mL). The resulting brown precipitate was filtered, washed with water and methanol and dried to provide 6-methyl-12,13-diallyl-12,13-dihydro-5,7-dioxo-6H-indolo[2,3-a]pyrrolo[3,4-c]carbazole Compound 4b (2.447 g, 83%) was obtained as an orange-brown solid. ¹H NMR (CDCl₃) δ 3.03 (s, 3H), 4.81 (s, 4H); 5.43 (t, 4H, J=18 Hz); 6.1 (m, 2H); 7.25-7.54 (m, 4H); 9.21 (d, 2H, J=8 Hz); ¹³C NMR δ23.91, 51.17, 111.76; 117.85; 120.33; 120.7; 122.03; 123.37; 125.9; 127.86; 132.96; 133.22; 145.28; 169.98; MS 861 (2M+Na), 442 (M+Na), 420 (M+H); HRMS Calcd. For C₂₇H₂₁N₃O₂: 419.1638. Found: 419.1623.

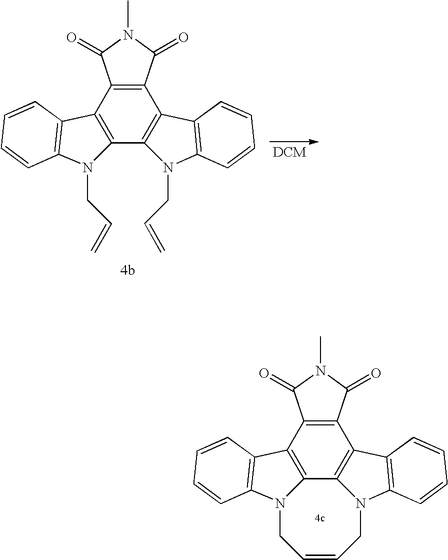

Compound 4b (1.26 g, 3 mmol) was dissolved in DCM (500 mL) and benzylidene-bis(tricyclohexylphosphine)dichlororuthenium (250 mg, 0.3 mmol) was added. The mixture was stirred at room temperature for 6 hrs. Silica gel (5 g) was added and the mixture was filtered over a thin pad of silica gel. The gel pad was washed sequentially with DCM (3×50 mL) and ethyl acetate (3×50 mL). The organic layers were combined and concentrated in vacuo. The resulting solids were washed with methanol, then filtered and dried to provide 6-methyl-12,13-(but-2-en-1,4-yl)-12,13-dihydro-5,7-dioxo-6H-indolo[2,3-a]pyrrolo[3,4-c]carbazole Compound 4c (1 g, 85%) as a brownish-yellow solid. ¹H NMR (CDCl₃) δ 2.91 (s, 3H); 4.77 (d, 4H, J=5 Hz); 6.21 (quin, 2H, J=4 Hz); 7.44 (t, 2H, J=5 Hz); 7.48 (d, 2H, J=7 Hz); 7.63 (t, 2H, J=6 Hz); 9.39 (d, 2H, J=6 Hz); ¹³C NMR δ 23.37, 39.35, 77.07, 108.18, 118.51, 119.03, 120.66, 121.32, 125.87, 126.83; 128.95, 131.43, 140.68, 169.35; MS: 805 (2M+Na), 414 (M+Na), 392 (M+H); HRMS Calcd. For C₂₅H₁₇N₃O₂: 391.1321. Found: 391.1331.

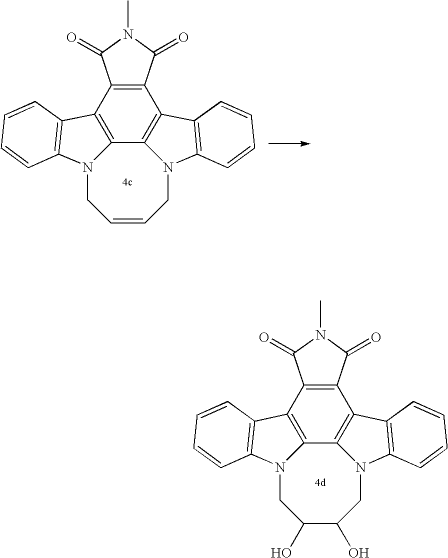

A mixture of Compound 4c (984 mg, 2.5 mmol), trimethylamine-N-oxide, (410 mg, 5.46 mmol) and anhydrous osmium trichloride (27 mg, 0.09 mmol) was diluted with chloroform (60 mL), tetrahydrofuran (30 mL) and water (5 drops). The mixture was stirred at room temperature for 24 hours. The solvent was removed in vacuo and the resulting solids were filtered off and washed with water, methanol and chloroform (4× each). The solids were dried in a vacuum oven to give 6-methyl-12,13-(2,3-dihydroxy-butan-1,4-yl)-12,13-dihydro-5,7-dioxo-6H-indolo[2,3-a]pyrrolo[3,4-c]carbazole Compound 4d (1.055 g, 99%) as an orange solid. ¹H NMR (d₆-DMSO, 400 MHz) δ 3.01 (s, 3H), 4.26 (s, 2H), 4.53 (d, 2H, J=13 Hz), 4.61 (d, 1H, J=9 Hz), 4.65 (d, 1H, J=9 Hz), 5.45 (s, 2H), 7.4 (t, 2H, J=8 Hz), 7.64 (t, 2H, J=8 Hz), 7.76 (d, 2H, J=8 Hz), 9.11 (d, 2H, J=8 Hz); ¹³C NMR (d₆-DMSO, 400 MHz) δ 24.39, 49.03, 70.51, 111.08, 118.26, 119.63, 121.52, 121.6, 125.23, 128.26, 170.16; MS (ES) 873 (2M+Na), 426 (M+H).

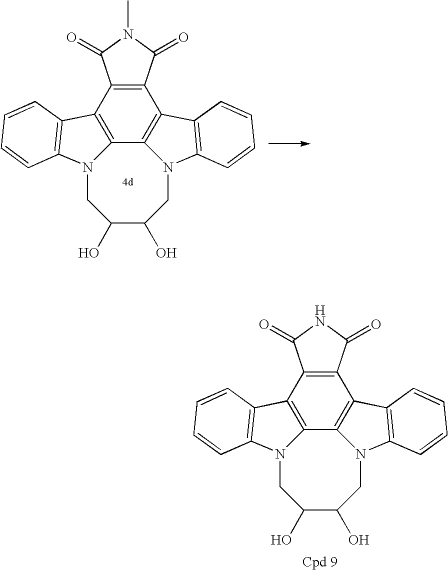

A mixture of Compound 4d (1 g, 2.35 mmol), KOH (30 g), 1,4-dioxane (20 mL) and absolute ethanol (100 mL) was heated at 105° C. for 5 days. The mixture was cooled to room temperature, diluted with H₂O and acidified with HCl (conc.) to pH 1. The mixture was extracted with ethyl acetate and the organic extracts were washed with water and brine solution. The aqueous layers were back extracted two times with ethyl acetate and the combined organic layers were concentrated in vacuo. The residue was mixed with 1,4-dioxane (50 mL), DMF (25 mL) and 1,1,3,3-hexamethyldisilazane (25 mL). The resulting mixture was heated to 95° C. for 36 hours and concentrated in vacuo. The residue was stirred with methanol (40 mL) and TFA (15 mL) and the resulting solids were filtered. The solids were sequentially washed with methanol and ethyl acetate (3× each) to provide Compound 9 (550 mg, 57%) as a yellow solid. ¹H NMR (d₆-DMSO, 300 MHz) δ 4.31 (bs, 2H), 4.59 (d, 2H, J=14 Hz), 4.71 (d, 1H, J=9 Hz), 4.76 (d, 1H, J=9 Hz), 5.45 (bs, 2H), 7.41 (t, 2H, J=8 Hz), 7.65 (t, 2H, J=8 Hz), 7.83 (d, 2H, J=8 Hz), 9.17 (d, 2H, J=8 Hz), 11.09 (s, 1H, NH); ¹³C NMR DEPT (d₆-DMSO, 400 MHz) δ 49.09 (CH₂), 70.55 (CH), 111.17 (CH), 121.58 (CH), 125.41 (CH), 128.24 (CH); MS (ES): 821 (2M−H), 410 (M−H); HRMS calcd. For C₂₄H₁₇N₃O₄: 411.1219. Found: 411.1207.

Using the procedure of Example 4, the following compounds were synthesized:

| Cpd | Name and Data |
|---|---|
| 1 | 12,13-(but-2-en-1,4-yl)-12,13-dihydro-5,7-dioxo-6H-indolo[2,3-a]pyrrolo[3,4- |
| | c]carbazole MS m/z 378(M+H) |
| 10 | 12,13-(2,3-dimethoxy-butan-1,4-yl)-12,13-dihydro-5,7-dioxo-6H-indolo[2,3- |
| | a]pyrrolo[3,4-c]carbazole MS m/z 440(M+H) |
| 11 | 12,13-(2,3-dihydroxy-pentan-1,5-yl)-12,13-dihydro-5,7-dioxo-6H-indolo[2,3- |
| | a]pyrrolo[3,4-c]carbazole |
| | ¹H NMR(300MHz, d₆-DMSO) δ 1.54(bs, 2H), 2.02(bs, 1H), 2.27(bs, 1H), 4.15(bs, |
| | 1H), 5.04(m, 3H), 5.12(m, 1H), 7.4(quart, 2H, J=7Hz), 7.65(m, 2H), 7.87(m, 2H), |
| | 9.38(d, 2H, J=8Hz), 11.09(s, 1H); MS m/z 841(2M+Na), 432(M+Na), 410(M+H) |

### Example 5

12,13-(2-hydroxy-butan-1,4-yl)-12,13-dihydro-5,7-dioxo-6H-indolo[2,3-a]pyrrolo[3,4-c]carbazole (Compound 7)

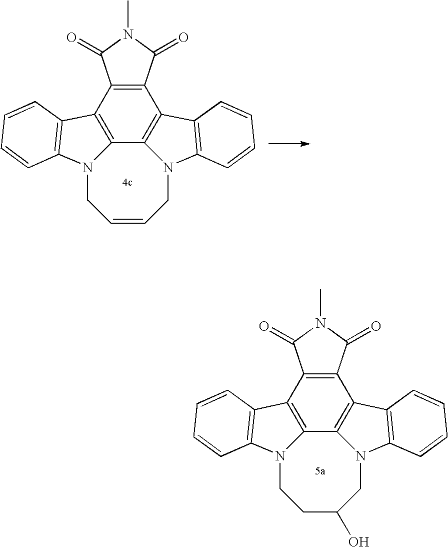

6-methyl-12,13-(but-2-en-1,4-yl)-12,13-dihydro-5,7-dioxo-6H-indolo[2,3-a]pyrrolo[3,4-c]carbazole Compound 4c (586 mg, 1.5 mmol) was slurried in THF (40 mL) and a solution of BH₃-THF (1M, 7.5 mL) was added at 0° C. over 10 minutes. The reaction mixture was stirred for 45 minutes at 0° C., then warmed to room temperature over 1 hour. The mixture was then cooled to 0° C., an aqueous H₂O₂ solution (50%, 35 mL) was added over 10 minutes, followed by a 10% aqueous NaOH solution (105 mL) added slowly over 20 minutes. The resulting mixture was stirred at 0° C. for 45 minutes, then extracted with ethyl acetate and washed twice with dilute NaOH solution. The aqueous layers were then back extracted with ethyl acetate. The organic layers were combined, dried (Na₂SO₄) and concentrated. The residue was diluted with methanol and the resulting solids were collected by filtration to give 6-methyl-12,13-(2-hydroxy-butan-1,4-yl)-12,13-dihydro-5,7-dioxo-6H-indolo[2,3-a]pyrrolo[3,4-c]carbazole Compound 5a (579 mg, 94%) as an orange solid. ¹H NMR (d₆-DMSO) δ 1.89 (m, 1H), 2.31 (m, 1H), 3.01 (s, 3H), 4.29 (m, 1H), 4.39-4.59 (m, 3H), 4.72 (dd, 1H, J=9, 14 Hz), 7.37 (t, 1H, J=7 Hz), 7.38 (t, 1H, J=7 Hz), 7.59 (t, 1H, J=7 Hz), 7.61 (t, 1H, J=7 Hz), 7.73 (d, 1H, J=9 Hz), 7.77 (d, 1H, J=9 Hz), 9.08 (d, 1H, J=8 Hz), 9.13 (d, 1H, J=8 Hz); MS 841 (2M+Na), 432 (M+Na), 410 (M+H).

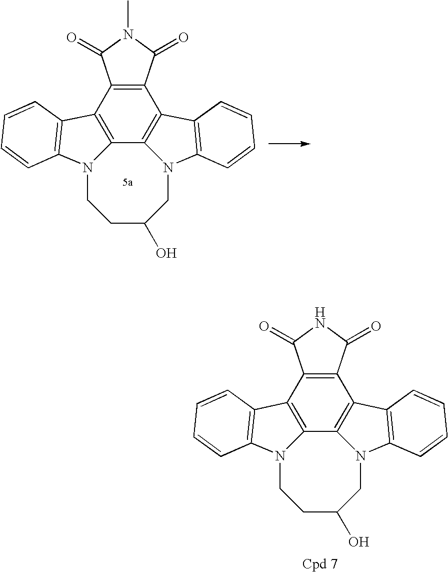

Compound 5a (579 mg, 1.43 mmol) was diluted with 1,4-dioxane (20 mL), EtOH (20 mL) and aqueous 30% KOH (40 mL). The mixture was heated to 95° C. for 7 days, then cooled and acidified with HCl (conc.) to pH 1. The mixture was then extracted with ethyl acetate and washed with H₂O and aqueous Na₂SO₄ solution. The aqueous layers were back extracted with ethyl acetate and the organic layers were combined, dried (Na₂SO₄) and concentrated. The resulting brownish residue was dissolved in DMF (80 mL) and heated with 1,1,3,3-hexamethyldisilazane (9 mL) and methanol (1 mL) for 4 hours. The mixture was cooled and an aqueous NaHCO₃ solution was added. The layers were separated and extracted with ethyl acetate. The organic extracts were evaporated and the residue was diluted with THF (100 mL), methanol (10 mL) and TFA (1 mL). The mixture was stirred for 2 hours at room temperature and the volatiles were removed in vacuo. The residue was diluted with methanol and filtered to provide Compound 7 (464 mg, 83%) as an orange solid. ¹H NMR (d₆-DMSO) δ 2.03 (bs, 1H), 2.43 (bs, 1H), 4.35 (bs, 1H), 4.53-4.72 (m, 3H), 4.84 (dd, 1H, J=9, 15 Hz), 5.44 (d, 1H, J=4 Hz), 7.4 (t, 1H, J=7 Hz), 7.41 (t, 1H, J=7 Hz), 7.64 (t, 1H, J=8 Hz), 7.65 (t, 1H, J=8 Hz), 7.82 (d, 1H, J=13 Hz), 7.85 (d, 1H, J=13 Hz), 9.16 (d, 1H, J=8 Hz), 9.22 (d, 1H, J=8 Hz), 11.08 (s, 1H); ¹³C NMR (d₆-DMSO) δ 25.8, 49.33, 67.68, 117.57, 118.5, 120.32, 120.99, 121.08, 121.55, 121.64, 125.01, 125.1, 127.66, 129.39, 131.7, 143.62, 171.39, 171.48; MS 396 (M+H), 394 (M−H); HRMS calcd. For C₂₄H₁₇N₃O₃: 395.1274. Found: 395.1270.

Using the procedure of Example 5, the following compounds were synthesized:

| Cpd | Name and Data |
|---|---|
| 8 | 12,13-(2-hydroxy-pentan-1,5-yl)-12,13-dihydro-5,7-dioxo-6H-indolo[2,3- |
| | a]pyrrolo[3,4-c]carbazole |
| | ¹H NMR(300MHz, d₆-DMSO) δ 1.54(bs, 2H), 2.02(bs, 1H), 2.27(bs, 1H), 4.15(bs, |
| | 1H), 5.04(m, 3H), 5.12(m, 1H), 7.4(quart, 2H, J=7Hz), 7.65(m, 2H), 7.87(m, 2H), |
| | 9.38(d, 2H, J=8Hz), 11.09(s, 1H); MS m/z 841(2M+Na), 432(M+Na), 410(M+H) |

### Example 6

12,13-(2-methoxycarbonyl-but-2-en-1,4-yl)-12,13-dihydro-5,7-dioxo-6H-indolo[2,3-a]pyrrolo[3,4-c]carbazole (Compound 3)
12,13-(2-methoxycarbonyl-2,3-dihydroxy-butan-1,4-yl)-12,13-dihydro-5,7-dioxo-6H-indolo[2,3-a]pyrrolo[3,4-c]carbazole (Compound 12)
12,13-(2-carboxy-2,3-dihydroxy-butan-1,4-yl)-12,13-dihydro-5,7-dioxo-6H-indolo[2,3-a]pyrrolo[3,4-c]carbazole (Compound 13)
12,13-{2-[(pyridin-4-ylmethyl)carbamoyl]-2,3-dihydroxy-butan-1,4-yl}-12,13-dihydro-5,7-dioxo-6H-indolo[2,3-a]pyrrolo[3,4-c]carbazole (Compound 117)

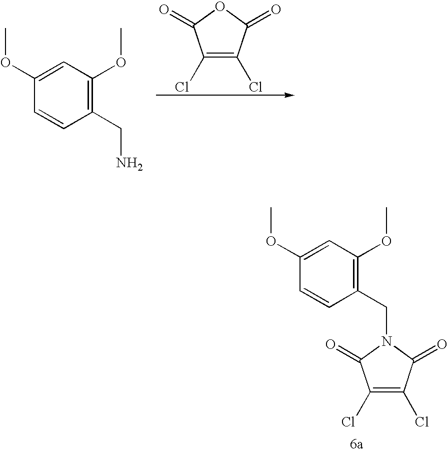

3,4-dichloro-furan-2,5-dione (6.7 g, 40 mmol) was mixed with 2,4-dimethoxy-benzylamine (6.25 mL) in glacial acetic acid (120 mL). The mixture was heated to 80° C. for 18 hrs. Upon cooling, the mixture was poured over ice and the precipitate was collected by filtration, then washed with water and NaHCO₃ (aq.) and dried in a vacuum oven to provide 3,4-dichloro-1-(2,4-dimethoxy-benzyl)-pyrrole-2,5-dione Compound 6a (11.08 g, 87%) as a light orange solid. ¹H NMR (d⁶-DMSO, 300 MHz) δ 3.73 (s, 3H), 3.76 (s, 3H), 4.54 (s, 2H), 6.44 (d, 1H, J=8 Hz), 6.57 (s, 1H), 7.12 (d, H, J=8 Hz); MS m/z 340 (M+2+Na), 338 (M+Na), 318 (M+2H), 316 (M+H).

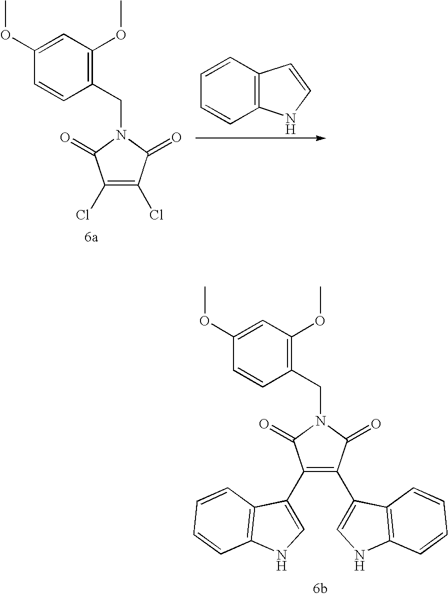

A mixture of ethyl magnesium bromide and indole (4 equivalents) were heated for 24 hrs. Upon cooling, the mixture was quenched with aqueous NH₄Cl and extracted with ethyl acetate. The organic layer washed with aqueous NH₄Cl and brine. The aqueous layer was back extracted and the organic extracts were combined and concentrated. The resulting residue was diluted with DCM, the solids were collected by filtration, then washed four times with DCM and dried in a vacuum oven to provide 1-(2,4-dimethoxy-benzyl)-3,4-bis-(1H-indol-3-yl)-pyrrole-2,5-dione Compound 6b (4.66 g, 56%). ¹H NMR (d⁶-DMSO, 300 MHz) δ 3.73 (s, 3H), 3.82 (s, 3H), 4.68 (s, 2H), 6.48 (d, 1H, J=8 Hz), 6.58 (s, 1H), 6.23 (t, 2H, J=8 Hz), 6.81 (d, 2H, J=9 Hz), 7.11 (quart, 3H, J=8 Hz), 7.38 (d, 2H, J=9 Hz), 7.85 (s, 2H), 11.68 (s, 2H); MS m/z/z 977 (2M+Na), 478 (M+H).

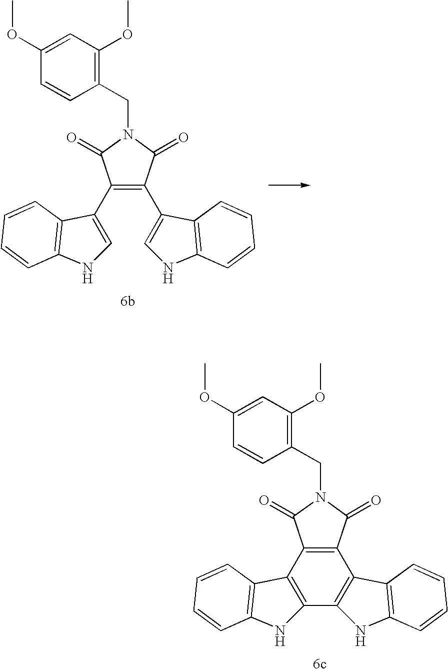

Compound 6b (1.7 g, 3.56 mmol) was dissolved in 1,4-dioxane (85 mL) and toluene (255 mL). The mixture was heated to 100° C. with stirring for 15 min., then DDQ (889 mg, 3.91 mmol) and p-toluenesulfonic acid (34 mg, 0.18 mmol) were added. The resulting mixture was stirred at 120° C. for 1 hr, then the heat was removed and the mixture was allowed to cool and was stirred at 20° C. overnight. After concentration, the residue was dissolved in ethyl acetate and washed with 5% aqueous NaHCO₃ and brine. After the layers were separated, the organic phase was concentrated to provide 6-(2,4-dimethoxy-benzyl)-12,13-dihydro-5,7-dioxo-6H-indolo[2,3-a]pyrrolo[3,4-c]carbazole Compound 6c (2.11 g) as a dark brown solid. ¹H NMR (d⁶-DMSO, 300 MHz) δ 3.72 (s, 3H), 3.86 (s, 3H), 4.82 (s, 2H), 6.44 (d, 1H, J=8 Hz), 6.60 (s, 1H), 7.08 (d, 1H, J=8 Hz), 7.34 (t, 2H, J=7 Hz), 7.58 (t, 2H, J=7 Hz), 7.80 (d, 2H, J=8 Hz), 8.90 (d, 2H, J=8 Hz), H), 11.97; MS m/z 973 (2M+Na), 489 (M+Na), 474 (M−H).

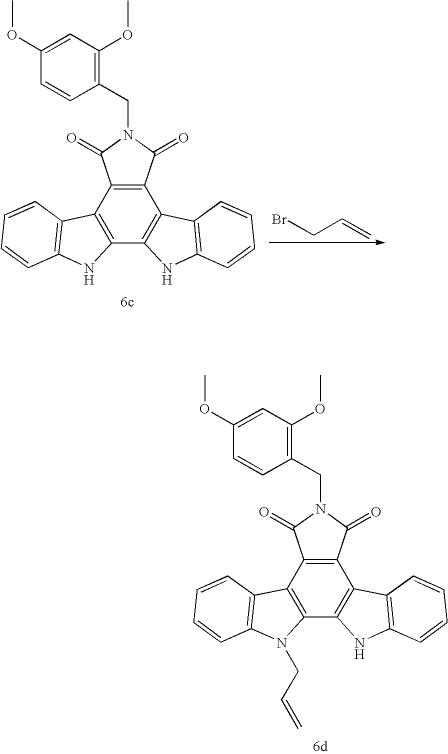

Compound 6c (3.3 g, 6.94 mmol) was dissolved in DMF and sodium hydride (555 mg, 13.88 mmol) was added. The mixture was stirred at 20° C. for 90 min, cooled at −52° C. for 1 hr and 3-bromo-prop-1-ene (600 μL, 6.94 mmol) was added. The mixture was stirred at −52° C. for 3 hrs, then at 20° C. overnight. The reaction was quenched with methanol and diluted with water. The mixture was extracted with ethyl acetate twice and the organic layers were washed with brine. The organic layers were combined and dried (Na₂SO₄), then concentrated and purified via flash column chromatography (CH₂Cl₂:MeOH=500:1) to provide 6-(2,4-dimethoxy-benzyl)-12-allyl-12,13-dihydro-5,7-dioxo-6H-indolo[2,3-a]pyrrolo[3,4-c]carbazole Compound 6d (1.82 g, 51% yield) as a brown solid. ¹H NMR (CDCl₃) 3.73 (s, 3H), 3.84 (s, 3H), 4.85 (s, 2H), 5.05-5.20 (m, 3H), 5.35 (d, 1H, J=10 Hz), 6.31 (m, 1H), 6.37 (d, 1H, H=8 Hz), 6.43 (s, 1H), 7.19 (d, 1H, J=8 Hz), 7.38 (m, 3H), 7.46-7.62 (m, 3H), 8.56 (s, 1H), 9.24 (t, 2H, J=9 Hz); MS m/z 1053 (2M+Na), 538 (M+Na), 516 (M+H).

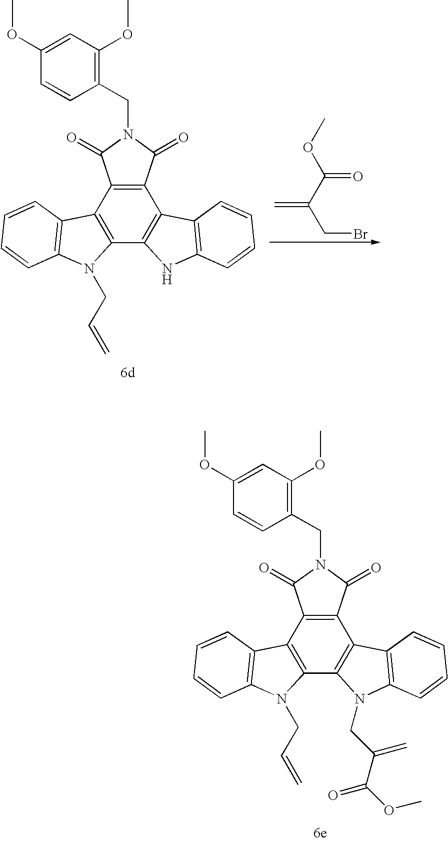

Compound 6d (1.53 g, 2.97 mmol) was dissolved in DMF (76 mL) and Cs₂CO₃ (2.42 g, 7.42 mmol) and (3-bromo-2-methylene-1-oxo-1-methoxy)propane (also referred to as 2-bromomethyl-acrylic acid methyl ester) (393 uL, 3.27 mmol) were added. The reaction mixture was stirred at 20° C. for 2 hrs and quenched with water (200 mL). The precipitate was collected by filtration, rinsed with methanol and dried in a vacuum oven to provide 6-(2,4-dimethoxy-benzyl)-12-allyl-13-[(2-methylene-3-oxo-3-methoxy)prop-1-yl]-12,13-dihydro-5,7-dioxo-6H-indolo[2,3-a]pyrrolo[3,4-c]carbazole Compound 6e (1.75 g, 96%) as a yellow solid. ¹H NMR<CDCl₃) 3.75 (s, 3H), 3.86 (s, 6H), 4.88 (s, 2H), 5.01 (s, 2H), 5.22 (s, 2H), 5.43 (m, 2H), 5.81 (s, 1H), 6.07 (m, 1H) 6.41 (d, 1H, J=8 Hz), 6.47 (s, 1H), 6.62 (s, 1H), 7.22 (s, 1H), 7.36 (d, 1H, J=7 Hz), 7.43 (m, 2H), 7.52 (m, 3H), 9.37 (t, 2H, J=7 Hz); MS m/z 1249 (2M+Na), 636 (M+Na), 614 (M+H).

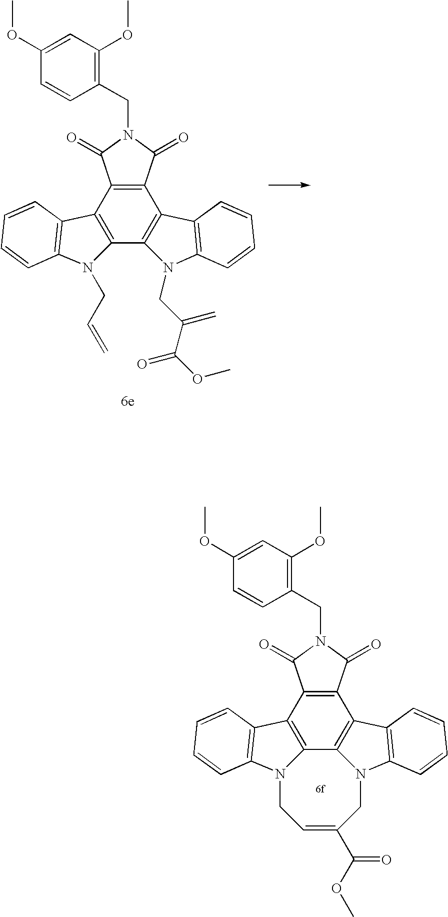

A mixture of Compound 6e (122.6 mg, 0.2 mmol), 1,3-bis-[(2,4,6-trimethylphenyl)-2-imidazolidinylidene]dichloro(phenylmethylene)-(tricyclohexylphosphine)ruthenium (21.2 mg, 0.025 mmol) and 1,2-dichloroethane (4 mL) was heated to 150° C. in a microwave oven for 30 min. The mixture was cooled to room temperature and the resulting precipitate was collected by filtration to provide 6-(2,4-dimethoxy-benzyl)-12,13-(2-methoxycarbonyl-but-2-en-1,4-yl)-12,13-dihydro-5,7-dioxo-6H-indolo[2,3-a]pyrrolo[3,4-c]carbazole Compound 6f (75 mg, 64%) as a yellow solid: ¹H NMR (CDCl₃) δ 3.62 (s, 3H), 3.73 (s, 3H), 3.86 (s, 3H), 4.77 (s, 2H), 5.62 (d, 2H, J=8 Hz), 5.7 (s, 2H), 6.43 (dd, 1H, J=2 Hz, J=8 Hz), 6.6 (d, 1H, J=2 Hz), 7.04 (d, 1H, J=8 Hz), 7.41 (t, 1H, J=8 Hz), 7.42 (t, 1H, J=8 Hz), 7.52 (t, 1H, J=8 Hz), 7.7 (m, 2H), 8.03 (d, 1H, J=5 Hz), 8.06 (d, 1H, J=5 Hz), 9.28 (d, 1H, J=5 Hz), 9.3 (d, 1H, J=5 Hz); MS m/z 585.9 (M+H), 584 (M−H); HRMS Calcd. For C₃₅H₂₇N₃O₆: 585.1899. Found: 585.1907.

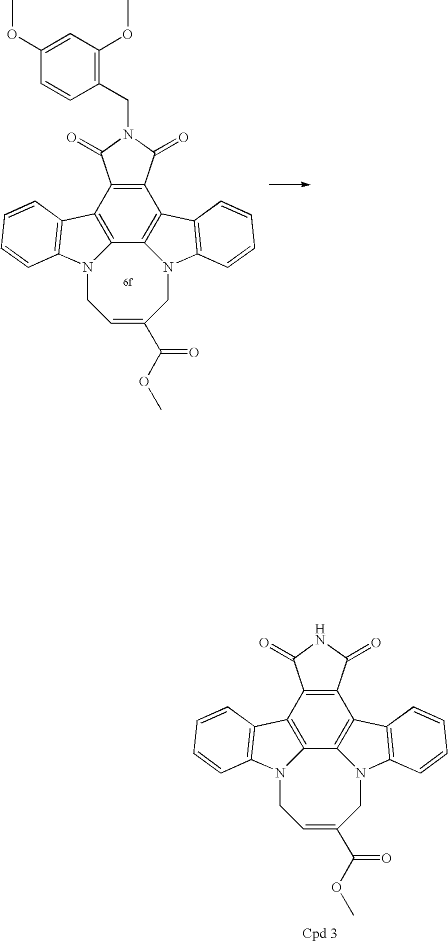

TFA (5.7 mL) and anisole (8 mL) were added to Compound 6f (155 mg, 0.265 mmol). The reaction mixture was heated to 90° C. for 8 hours, cooled and concentrated. The residue was mixed with hexane and the resulting solids were filtered, washed with hexane three times and dried under vacuum. The orange solid was dissolved in THF, filtered and the filtrate was concentrated to dryness. The resulting solids were diluted with methanol, filtered, washed with methanol and dried to provide Compound 3 (82 mg, 71%) as a yellow solid. ¹H NMR (CDCl₃) δ 3.63 (s, 3H), 5.68 (d, 2H, J=8 Hz), 5.76 (s, 2H), 7.42 (t, 1H, J=8 Hz), 7.43 (t, 1H, J=8 Hz), 7.53 (t, 1H, J=8 Hz), 7.69 (t, 1H, J=8 Hz), 7.7 (t, 1H, J=8 Hz), 8.05 (d, 1H, J=4 Hz), 8.08 (d, 1H, J=4 Hz), 9.33 (t, 2H, J=7 Hz), 11.17 (s, 1H); MS m/z 893 (2M+Na), 436 (M+H); HRMS Calcd. For C₂₆H₁₇N₃O₄: 435.1219. Found: 435.1209.

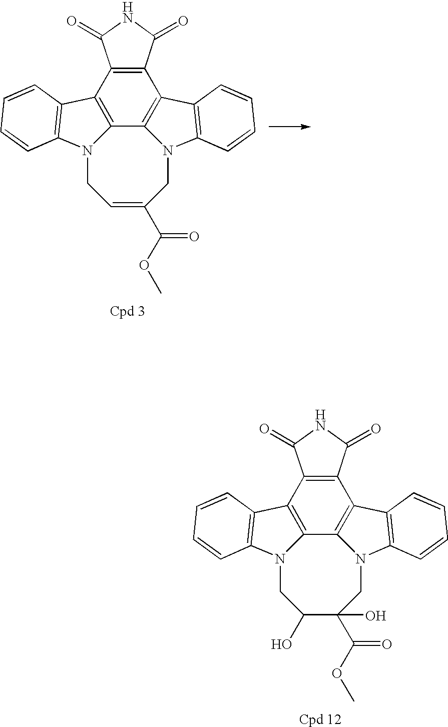

Compound 3 (70 mg, 0.161 mmol) was suspended in CHCl₃ (12 mL) and THF (6 mL). Trimethylamine-N-oxide (30 mg), OsCl₃ (8 mg) and H₂O (8 drops) were added. The mixture was stirred at room temperature for 7 hours and the solvent was removed under vacuum. The resulting residue was stirred with water, then filtered and washed with water, methanol and chloroform (twice each). The solids were dried under vacuum to provide Compound 12 (61 mg, 81%) as an orange solid. ¹H NMR (CDCl₃): δ 3.47 (s, 3H), 4.6 (m, 2H), 4.8 (quart, 2H, J=14 Hz), 5.09 (quart, 1H, J=6 Hz), 5.82 (d, 1H, J=5 Hz), 6.31 (s, 1H), 7.42 (quart, 2H, J=8 Hz), 7.67 (quint, 2H, J=7 Hz), 7.88 (d, 1H, J=8 Hz), 9.18 (d, 1H, J=8 Hz), 9.26 (d, 1H, J=8 Hz), 11.15 (s, 1H); MS m/z 961 (2M+Na), 470 (M+H), 468 (M−H); HRMS Calcd. For C₂₆H₁₉N₃O₆: 469.1273. Found: 469.1288.

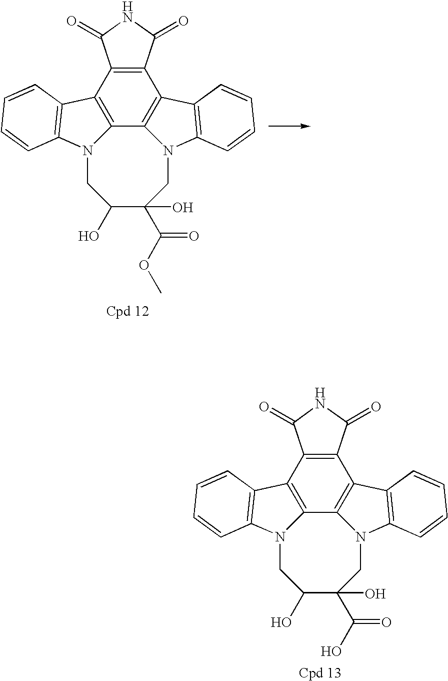

Compound 12 (50 mg, 0.106 mmol) was suspended in THf (5 mL) and H₂O (2.5 mL) and LiOH (5.2 mg) were added. The mixture was stirred at room temperature for 2 hours and then concentrated under vacuum. The residue was diluted with water and extracted with ethyl acetate. The mixture was acidified with a 1N HCl solution. The organic layer washed with a brine solution, then dried over Na₂SO₄ and concentrated. The residue was dried under vacuum to provide Compound 13 (36 mg (75%). ¹H NMR (CDCl₃): δ 4.57 (d, 2H, J=7 Hz), 4.66 (d, 1H, J=16 Hz), 4.84 (d, 1H, J=16 Hz), 5.06 (m, 1H), 7.38 (t, 1H, J=8 Hz), 7.42 (t, 1H, J=8 Hz), 7.61 (m, 2H), 7.68 (d, 1H, J=7 Hz), 7.85 (d, 1H, J=8 Hz), 9.16 (d, 1H, J=8 Hz), 9.24 (d, 1H, J=8 Hz), 11.12 (s, 1H); MS m/z 933 (2M+Na), 456 (M+H), 454 (M−H); HRMS Calcd. For C₂₅H₁₇N₃O₆: 455.1117. Found: 455.1138.

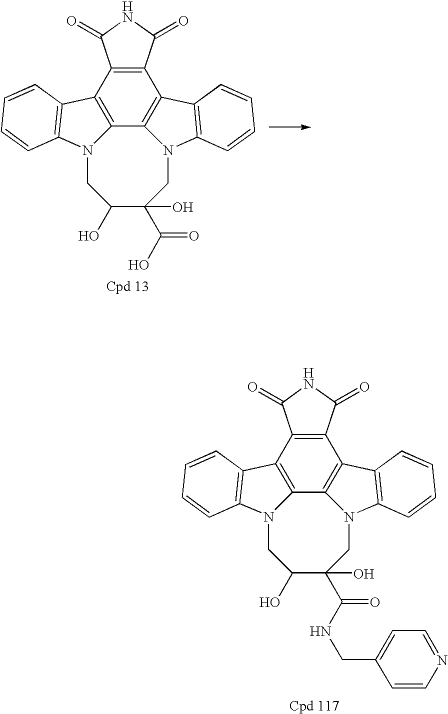

4-aminomethylpyridine (2 eq), N-hydroxy-benzotriazole (2 eq), N,N-dimethylaminopropyl-ethyl carbodiimide hydrochloride (2 eq) and diisopropyl ethylamine (2 eq) were sequentially added to a solution of Compound 13 (18 mg, 0.04 mmol) in DMF (2 mL). The mixture was stirred at room temperature overnight. The reaction product was isolated by reverse phase HPLC to provide Compound 117 (12 mg, 55%). ¹H NMR (d⁶-DMSO, 300 MHz) δ 4.47 (d, 2H, J=5.7 Hz), 4.71 (d, 3H, J=6 Hz), 4.80 (d, 1H, J=16 Hz), 5.07 (m, 1H), 7.35 (t, 1H, J=7 Hz), 7.44 (t, 2H, J=7 Hz), 7.70 (q, 3H, J=7 Hz), 7.87 (d, 1H, J=8 Hz), 8.7 (s, 2H), 8.9 (s, 1H), 9.15 (d, 1H, J=8 Hz), 9.25 (d, 1H, J=7 Hz), 11.12 (s, 1H); MS m/z 1091 (2M+H), 568 (M+Na), 546 (M+H).

Using the procedure of Example 6, the following compounds were synthesized:

| Cpd | Name and Data |
|---|---|
| 118 | 12,13-{2-[(1,3-hydroxy-isopropyl)carbamoyl]-2,3-dihydroxy-butan-1,4-yl}- |
| | 12,13-dihydro-5,7-dioxo-6H-indolo[2,3-a]pyrrolo[3,4-c]carbazole MS m/z |
| | 529(M+H) |
| 119 | 12,13-{2-[(3-dimethylamino-pyrrolidin-1-yl)carbonyl]-2,3-dihydroxy-butan-1,4- |
| | yl}-12,13-dihydro-5,7-dioxo-6H-indolo[2,3-a]pyrrolo[3,4-c]carbazole MS m/z |
| | 1125(2M+Na), 552(M+H) |
| 120 | 12,13-{2-[(3-morpholin-4-yl-prop-1-yl)carbamoyl]-2,3-dihydroxy-butan-1,4-yl}- |
| | 12,13-dihydro-5,7-dioxo-6H-indolo[2,3-a]pyrrolo[3,4-c]carbazole MS m/z |
| | 1185(2M+Na), 582(M+H) |
| 121 | 12,13-[2-(morpholin-4-yl-carbonyl)-2,3-dihydroxy-butan-1,4-yl]-12,13-dihydro- |
| | 5,7-dioxo-6H-indolo[2,3-a]pyrrolo[3,4-c]carbazole MS m/z 1071(2M+Na), |
| | 523(M−H) |
| 122 | 12,13-{2-[(2-oxo-tetrahydro-furan-3-yl)carbamoyl]-2,3-dihydroxy-butan-1,4-yl}- |
| | 12,13-dihydro-5,7-dioxo-6H-indolo[2,3-a]pyrrolo[3,4-c]carbazole MS m/z |
| | 1099(2M+Na), 561(M+Na), 539(M+H) |
| 123 | 12,13-[2-(isopropyl-carbamoyl)-2,3-dihydroxy-butan-1,4-yl]-12,13-dihydro-5,7- |
| | dioxo-6H-indolo[2,3-a]pyrrolo[3,4-c]carbazole MS m/z 993(2M+H), |
| | 497(M+H) |
| 124 | 12,13-{2-[(2-methoxy-ethyl)carbamoyl]-2,3-dihydroxy-butan-1,4-yl}-12,13- |
| | dihydro-5,7-dioxo-6H-indolo[2,3-a]pyrrolo[3,4-c]carbazole MS m/z |
| | 1047(2M+Na), 535(M+Na), 513(M+H) |
| 125 | 12,13-{2-[(4-methyl-piperazin-1-yl)carbonyl]-2,3-dihydroxy-butan-1,4-yl}- |
| | 12,13-dihydro-5,7-dioxo-6H-indolo[2,3-a]pyrrolo[3,4-c]carbazole MS m/z |
| | 1097(2M+Na), 1075(2M+H), 538(M+H) |
| 126 | 12,13-(2-{[4-(3-dimethylamino-prop-1-yl)piperazin-1-yl]carbonyl}-2,3- |
| | dihydroxy-butan-1,4-yl)-12,13-dihydro-5,7-dioxo-6H-indolo[2,3-a]pyrrolo[3,4- |
| | c]carbazole MS m/z 609(M+H) |
| 127 | 12,13-(2-{[3-(2-oxo-pyrrolidin-1-yl)prop-1-yl]carbamoyl}-2,3-dihydroxy-butan- |
| | 1,4-yl)-12,13-dihydro-5,7-dioxo-6H-indolo[2,3-a]pyrrolo[3,4-c]carbazole MS |
| | m/z 1181(2M+Na), 1159(2M+H), 602(M+Na), 580(M+H) |
| 128 | 12,13-{2-[(2-thien-2-yl-ethyl)carbamoyl]-2,3-dihydroxy-butan-1,4-yl}-12,13- |
| | dihydro-5,7-dioxo-6H-indolo[2,3-a]pyrrolo[3,4-c]carbazole MS m/z |
| | 1151(2M+Na), 565(M+H) |
| 129 | 12,13-(2-{[4-(4-hydroxy-phenyl)piperazin-1-yl]carbonyl}-2,3-dihydroxy-butan- |
| | 1,4-yl)-12,13-dihydro-5,7-dioxo-6H-indolo[2,3-a]pyrrolo[3,4-c]carbazole MS |
| | m/z 616(M+H) |
| 130 | 12,13-(2-{[(4-(2-hydroxy-ethyl)piperazin-1-yl]carbonyl}-2,3-dihydroxy-butan- |
| | 1,4-yl)-12,13-dihydro-5,7-dioxo-6H-indolo[2,3-a]pyrrolo[3,4-c]carbazole MS |
| | m/z 1157(2M+Na), 590(M+Na), 568(M+H) |
| 131 | 12,13-{2-[(4-pyridin-2-yl-piperazin-1-yl)carbonyl]-2,3-dihydroxy-butan-1,4-yl}- |
| | 12,13-dihydro-5,7-dioxo-6H-indolo[2,3-a]pyrrolo[3,4-c]carbazole MS m/z |
| | 1223(2M+Na), 601(M+H) |
| 132 | 12,13-{2-[(4-hydroxy-piperidin-1-yl)carbonyl]-2,3-dihydroxy-butan-1,4-yl}- |
| | 12,13-dihydro-5,7-dioxo-6H-indolo[2,3-a]pyrrolo[3,4-c]carbazole MS m/z |
| | 561(M+Na), 539(M+H), 537(M−H) |
| 133 | 12,13-(2-{[4-(pyrrolidin-1-yl-carbonylmethyl)piperazin-1-yl]carbonyl}-2,3- |
| | dihydroxy-butan-1,4-yl)-12,13-dihydro-5,7-dioxo-6H-indolo[2,3-a]pyrrolo[3,4- |
| | c]carbazole MS m/z 657(M+Na), 635(M+H) |
| 134 | 12,13-(2-{[4-(2-morpholin-4-yl-ethyl)piperazin-1-yl]carbonyl}-2,3-dihydroxy- |
| | butan-1,4-yl)-12,13-dihydro-5,7-dioxo-6H-indolo[2,3-a]pyrrolo[3,4-c]carbazole |
| | MS m/z 637(M+H) |

### Example 7

12,13-{2-[(2-dimethylamino-ethyl)carbamoyloxy]-butan-1, 4-yl}-12,13-dihydro-5,7-dioxo-6H-indolo[2,3-a]pyrrolo[3,4-c]carbazole (Compound 50)

12,13-(2-hydroxy-butan-1,4-yl)-12,13-dihydro-5,7-dioxo-6H-indolo[2,3-a]pyrrolo[3,4-c]carbazole Compound 7 (1.1 g) was mixed with DMF (10 mL) and THF (100 mL), then CDI (3.2 g) and DMAP (560 mg) were added. The reaction mixture was stirred for 24 hours, followed by precipitation from water and filtration. The solids were washed with additional water then dried in a vacuum oven to give a crude product (565 mg) as a yellow solid, which was used in the next step without further purification.

N,N-dimethylethylene diamine (110 μL) was added to a solution of the crude product (50 mg, approximately 50% by weight, 0.05 mmol) in methylsulfoxide (4 mL). The reaction mixture was heated to 60° C. for 20 hours, then cooled and extracted with ethyl acetate. The organic layer washed with water and a Na₂SO₄ (aq.) solution. The layers were separated and the organic phase was dried over Na₂SO₄, then concentrated and purified by reverse phase HPLC. The solvent was removed via freeze drying to provide Compound 50 (16 mg, 63%) as a yellow solid. HRMS: Calcd. for C₂₉H₂₈N₅O₄ (M+H): 510.2141. Found: 510.2123.

Using the procedure of Example 7, the following compounds were synthesized:

| Cpd | Name and Data |
|---|---|
| 36 | 12,13-{2-[(3-imidazol-1-yl-prop-1-yl)carbamoyloxy]-butan-1,4-yl}-12,13-dihydro- |
| | 5,7-dioxo-6H-indolo[2,3-a]pyrrolo[3,4-c]carbazole MS m/z 569(M+Na), |
| | 547(M+H) |
| 37 | 12,13-{2-[(t-butoxycarbonylmethyl)carbamoyloxy]-butan-1,4-yl}-12,13-dihydro- |
| | 5,7-dioxo-6H-indolo[2,3-a]pyrrolo[3,4-c]carbazole MS m/z 1015(2M+Na), |
| | 519(M+Na), 497(M+H) |
| 38 | 12,13-[2-(prop-1-ylcarbamoyloxy)-butan-1,4-yl]-12,13-dihydro-5,7-dioxo-6H- |
| | indolo[2,3-a]pyrrolo[3,4-c]carbazole MS m/z 503(M+Na), 481(M+H) |
| 39 | 12,13-[2-(prop-2-ylcarbamoyloxy)-butan-1,4-yl]-12,13-dihydro-5,7-dioxo-6H- |
| | indolo[2,3-a]pyrrolo[3,4-c]carbazole MS m/z 503(M+Na), 481(M+H) |
| 40 | 12,13-[2-(t-butylcarbamoyloxy)-butan-1,4-yl]-12,13-dihydro-5,7-dioxo-6H- |
| | indolo[2,3-a]pyrrolo[3,4-c]carbazole MS m/z 517(M+Na), 495(M+H) |
| 41 | 12,13-{2-[(2-methoxy-ethyl)carbamoyloxy]-butan-1,4-yl}-12,13-dihydro-5,7- |
| | dioxo-6H-indolo[2,3-a]pyrrolo[3,4-c]carbazole MS m/z 519(M+Na), 497(M+H) |
| 42 | 12,13-{2-[(3-morpholin-4-yl-prop-1-yl)carbamoyloxy]-butan-1,4-yl}-12,13- |
| | dihydro-5,7-dioxo-6H-indolo[2,3-a]pyrrolo[3,4-c]carbazole MS m/z 566(M+H) |
| 43 | 12,13-(2-{[3-(4-methyl-piperazin-1-yl)prop-1-yl]carbamoyloxy}-butan-1,4-yl)- |
| | 12,13-dihydro-5,7-dioxo-6H-indolo[2,3-a]pyrrolo[3,4-c]carbazole MS m/z |
| | 579(M+H) |
| 44 | 12,13-{2-[(4-benzyl-piperazin-1-yl)carbonyloxy]-butan-1,4-yl}-12,13-dihydro-5,7- |
| | dioxo-6H-indolo[2,3-a]pyrrolo[3,4-c]carbazole MS m/z 612(M+H) |
| 45 | 12,13-{2-[(4-methyl-benzyl)carbamoyloxy]-butan-1,4-yl}-12,13-dihydro-5,7- |
| | dioxo-6H-indolo[2,3-a]pyrrolo[3,4-c]carbazole MS m/z 565(M+Na), 543(M+H) |
| 46 | 12,13-{2-[(benzo[1,3]dioxol-5-yl-methyl)carbamoyloxy]-butan-1,4-yl}-12,13- |
| | dihydro-5,7-dioxo-6H-indolo[2,3-a]pyrrolo[3,4-c]carbazole MS m/z 571(M−H) |
| 47 | 12,13-{2-[(pyridin-4-yl-methyl)carbamoyloxy]-butan-1,4-yl}-12,13-dihydro-5,7- |
| | dioxo-6H-indolo[2,3-a]pyrrolo[3,4-c]carbazole MS m/z 530(M+H) |
| 48 | 12,13-(2-{[(5-methyl-furan-2-yl)methyl]carbamoyloxy}-butan-1,4-yl)-12,13- |
| | dihydro-5,7-dioxo-6H-indolo[2,3-a]pyrrolo[3,4-c]carbazole MS m/z 555(M+Na), |
| | 533(M+H) |
| 49 | 12,13-(2-{[2-(3,4-dimethoxy-phenyl)ethyl]carbamoyloxy}-butan-1,4-yl)-12,13- |
| | dihydro-5,7-dioxo-6H-indolo[2,3-a]pyrrolo[3,4-c]carbazole MS m/z 601(M−H) |
| 51 | 12,13-{2-[(4-methyl-piperazin-1-yl)carbonyloxy]-butan-1,4-yl}-12,13-dihydro-5,7- |
| | dioxo-6H-indolo[2,3-a]pyrrolo[3,4-c]carbazole MS m/z 522(M+H) |
| 52 | 12,13-(2-{[2-(pyrrolidin-1-yl-methyl)pyrrolidin-1-yl]carbonyloxy}-butan-1,4-yl)- |
| | 12,13-dihydro-5,7-dioxo-6H-indolo[2,3-a]pyrrolo[3,4-c]carbazole MS m/z |
| | 576(M+H) |
| 53 | 12,13-{2-[(4-cyclohexyl-piperazin-1-yl)carbonyloxy]-butan-1,4-yl}-12,13- |
| | dihydro-5,7-dioxo-6H-indolo[2,3-a]pyrrolo[3,4-c]carbazole MS m/z 590(M+H) |
| 54 | 12,13-(2-{[4-(benzo[1,3]dioxol-5-yl-methyl)piperazin-1-yl]carbonyloxy}-butan- |
| | 1,4-yl)-12,13-dihydro-5,7-dioxo-6H-indolo[2,3-a]pyrrolo[3,4-c]carbazole MS m/z |
| | 642(M+H) |
| 55 | 12,13-{2-[(4-pyridin-4-yl-piperazin-1-yl)carbonyloxy]-butan-1,4-yl}-12,13- |
| | dihydro-5,7-dioxo-6H-indolo[2,3-a]pyrrolo[3,4-c]carbazole MS m/z 585(M+H) |
| 56 | 12,13-(2-{[4-(2-morpholin-4-yl-ethyl)piperazin-1-yl]carbonyloxy}-butan-1,4-yl)- |
| | 12,13-dihydro-5,7-dioxo-6H-indolo[2,3-a]pyrrolo[3,4-c]carbazole MS m/z |
| | 621(M+H) |
| 57 | 12,13-[2-({4-[2-(2-oxo-pyrrolidin-1-yl)ethyl]piperazin-1-yl}carbonyloxy)-butan- |
| | 1,4-yl]-12,13-dihydro-5,7-dioxo-6H-indolo[2,3-a]pyrrolo[3,4-c]carbazole MS m/z |
| | 1259(2M+Na), 619(M+H) |
| 58 | 12,13-(2-{[4-(4-hydroxy-phenyl)piperazin-1-yl]carbonyloxy}-butan-1,4-yl)-12,13- |
| | dihydro-5,7-dioxo-6H-indolo[2,3-a]pyrrolo[3,4-c]carbazole MS m/z |
| | 1221(2M+Na), 600(M+H) |
| 59 | 12,13-(2-{[4-(4-methylcarbonyl-phenyl)piperazin-1-yl]carbonyloxy}-butan-1,4- |
| | yl)-12,13-dihydro-5,7-dioxo-6H-indolo[2,3-a]pyrrolo[3,4-c]carbazole MS m/z |
| | 1273(2M+Na), 648(M+Na), 625(M+H) |
| 60 | 12,13-{2-[(hexahydro-1H-1,4-diazepin-1-yl)carbonyloxy]-butan-1,4-yl}-12,13- |
| | dihydro-5,7-dioxo-6H-indolo[2,3-a]pyrrolo[3,4-c]carbazole MS m/z 522(M+H) |
| 61 | 12,13-{2-[N-methyl-N-(1-benzyl-pyrrolidin-3-yl)carbamoyloxy]-butan-1,4-yl}- |
| | 12,13-dihydro-5,7-dioxo-6H-indolo[2,3-a]pyrrolo[3,4-c]carbazole MS m/z |
| | 634(M+Na), 612(M+H) |
| 62 | 12,13-{2-[(4-benzhydryl-piperazin-1-yl)carbonyloxy]-butan-1,4-yl}-12,13- |
| | dihydro-5,7-dioxo-6H-indolo[2,3-a]pyrrolo[3,4-c]carbazole MS m/z 674(M+H) |
| 63 | 12,13-{2-[(4-pyridin-2-yl-piperazin-1-yl)carbonyloxy]-butan-1,4-yl}-12,13- |
| | dihydro-5,7-dioxo-6H-indolo[2,3-a]pyrrolo[3,4-c]carbazole MS m/z 585(M+H) |
| 64 | 12,13-{2-[(4-phenyl-piperazin-1-yl)carbonyloxy]-butan-1,4-yl}-12,13-dihydro-5,7- |
| | dioxo-6H-indolo[2,3-a]pyrrolo[3,4-c]carbazole MS m/z 584(M+H) |
| 65 | 12,13-(2-{[4-(2-phenyl-ethyl)piperazin-1-yl]carbonyloxy}-butan-1,4-yl)-12,13- |
| | dihydro-5,7-dioxo-6H-indolo[2,3-a]pyrrolo[3,4-c]carbazole MS m/z 612(M+H) |
| 66 | 12,13-(2-{[4-(2-hydroxy-ethyl)piperazin-1-yl]carbonyloxy}-butan-1,4-yl)-12,13- |
| | dihydro-5,7-dioxo-6H-indolo[2,3-a]pyrrolo[3,4-c]carbazole MS m/z 552(M+H) |
| 67 | 12,13-(2-{[N-methyl-N-(2-dimethylamino-ethyl)]carbamoyloxy}-butan-1,4-yl)- |
| | 12,13-dihydro-5,7-dioxo-6H-indolo[2,3-a]pyrrolo[3,4-c]carbazole MS m/z |
| | 523(M+H) |
| 68 | 12,13-(2-{[4-(3-dimethylamino-prop-1-yl)piperazin-1-yl]carbonyloxy}-butan-1,4- |
| | yl)-12,13-dihydro-5,7-dioxo-6H-indolo[2,3-a]pyrrolo[3,4-c]carbazole MS m/z |
| | 593(M+H) |
| 70 | 12,13-(2-{[4-(2-methoxy-phenyl)piperazin-1-yl]carbonyloxy}-butan-1,4-yl)-12,13- |
| | dihydro-5,7-dioxo-6H-indolo[2,3-a]pyrrolo[3,4-c]carbazole MS m/z 614(M+H) |
| 71 | 12,13-[2-(morpholin-4-yl-carbonyloxy)-butan-1,4-yl]-12,13-dihydro-5,7-dioxo- |
| | 6H-indolo[2,3-a]pyrrolo[3,4-c]carbazole MS m/z 507(M−H) |
| 72 | 12,13-[2-(pyrrolidin-3-yl-carbamoyloxy)-butan-1,4-yl]-12,13-dihydro-5,7-dioxo- |
| | 6H-indolo[2,3-a]pyrrolo[3,4-c]carbazole MS m/z 508(M+H) |
| 73 | 12,13-(2-{[(3S)-3-dimethylamino-pyrrolidin-1-yl]carbonyloxy}-butan-1,4-yl)- |
| | 12,13-dihydro-5,7-dioxo-6H-indolo[2,3-a]pyrrolo[3,4-c]carbazole MS m/z |
| | 536(M+H) |
| 74 | 12,13-(2-{[(3R)-3-dimethylamino-pyrrolidin-1-yl]carbonyloxy}-butan-1,4-yl)- |
| | 12,13-dihydro-5,7-dioxo-6H-indolo[2,3-a]pyrrolo[3,4-c]carbazole MS m/z |
| | 536(M+H) |
| 75 | 12,13-[2-(piperidin-4-yl-carbamoyloxy)-butan-1,4-yl]-12,13-dihydro-5,7-dioxo- |
| | 6H-indolo[2,3-a]pyrrolo[3,4-c]carbazole MS m/z 522(M+H) |
| 114 | 12,13-{2-[(2-dimethylamino-ethyl)carbamoyloxy]-pentan-1,5-yl}-12,13-dihydro- |
| | 5,7-dioxo-6H-indolo[2,3-a]pyrrolo[3,4-c]carbazole MS m/z 524(M+H) |
| 115 | 12,13-(2-{[4-(3-dimethylamino-prop-1-yl)piperazin-1-y]carbonyloxy}-pentan-1,5- |
| | yl)-12,13-dihydro-5,7-dioxo-6H-indolo[2,3-a]pyrrolo[3,4-c]carbazole MS m/z |
| | 607(M+H) |
| 116 | 12,13-(2-{[3-(4-methyl-piperazin-1-yl)prop-1-yl]carbamoyloxy}-pentan-1,5-yl)- |
| | 12,13-dihydro-5,7-dioxo-6H-indolo[2,3-a]pyrrolo[3,4-c]carbazole MS m/z |
| | 593(M+H) |
| 154 | 12,13-{2-[(2-methoxy-ethyl)carbamoyloxymethyl]propan-1,3-yl]-6,7,12,13- |
| | tetrahydro-5-oxo-5H-indolo[2,3-a]pyrrolo[3,4-c]carbazole MS m/z 483(M+H) |
| 155 | 12,13-(isopropyl-carbamoyloxymethyl)-propan-1,3-yl)-6,7,12,13-tetrahydro-5- |
| | oxo-5H-indolo[2,3-a]pyrrolo[3,4-c]carbazole MS m/z 467(M+H) |
| 156 | 12,13-(2-{[(5-methyl-furan-2-yl)methyl]carbamoyloxymethyl}-propan-1,3-yl)- |
| | 6,7,12,13-tetrahydro-5-oxo-5H-indolo[2,3-a]pyrrolo[3,4-c]carbazole MS m/z |
| | 541(M+Na), 529(M+H) |
| 157 | 12,13-(2-{[3-(2-oxo-pyrrolidin-1-yl)prop-1-yl]carbamoyloxymethyl}-propan-1,3- |
| | yl)-6,7,12,13-tetrahydro-5-oxo-5H-indolo[2,3-a]pyrrolo[3,4-c]carbazole MS m/z |
| | 572(M+Na), 550(M+H) |
| 158 | 12,13-(2-{[4-(4-hydroxy-phenyl)piperazin-1-yl]carbonyloxymethyl}-propan-1,3- |
| | yl)-6,7,12,13-tetrahydro-5-oxo-5H-indolo[2,3-a]pyrrolo[3,4-c]carbazole MS m/z |
| | 586(M+H) |
| 159 | 12,13-{2-[(4-hydroxy-piperidin-1-yl)carbonyloxymethyl]-propan-1,3-yl}- |
| | 6,7,12,13-tetrahydro-5-oxo-5H-indolo[2,3-a]pyrrolo[3,4-c]carbazole MS m/z |
| | 509(M+H) |
| 160 | 12,13-{2-[(2-pyridin-2-yl-ethyl)carbamoyloxymethyl]-propan-1,3-yl}-6,7,12,13- |
| | tetrahydro-5-oxo-5H-indolo[2,3-a]pyrrolo[3,4-c]carbazole MS m/z 552(M+Na), |
| | 530(M+H) |
| 161 | 12,13-(2-{[2-(3,4-dimethoxy-phenyl)ethyl]carbamoyloxymethyl}-propan-1,3-yl)- |
| | 6,7,12,13-tetrahydro-5-oxo-5H-indolo[2,3-a]pyrrolo[3,4-c]carbazole MS m/z |
| | 611(M+Na), 589(M+H) |
| 162 | 12,13-{2-[(4-pyrrolidin-1-yl-piperidin-1-yl)carbonyloxymethyl]-propan-1,3-yl}- |
| | 6,7,12,13-tetrahydro-5-oxo-5H-indolo[2,3-a]pyrrolo[3,4-c]carbazole MS m/z |
| | 562(M+H) |
| 163 | 12,13-{2-[(1-benzyl-piperidin-4-yl)carbamoyloxymethyl]-propan-1,3-yl}- |
| | 6,7,12,13-tetrahydro-5-oxo-5H-indolo[2,3-a]pyrrolo[3,4-c]carbazole MS m/z |
| | 598(M+H) |
| 164 | 12,13-(2-{[4-(2-hydroxy-ethyl)piperazin-1-yl]carbonyloxymethyl}-propan-1,3-yl)- |
| | 6,7,12,13-tetrahydro-5-oxo-5H-indolo[2,3-a]pyrrolo[3,4-c]carbazole MS m/z |
| | 560(M+Na), 538(M+H) |
| 165 | 12,13-{2-[(3-morpholin-4-yl-prop-1-yl)carbamoyloxymethyl]-propan-1,3-yl}- |
| | 6,7,12,13-tetrahydro-5-oxo-5H-indolo[2,3-a]pyrrolo[3,4-c]carbazole MS m/z |
| | 552(M+H) |
| 166 | 12,13-[2-({4-[2-(2-hydroxy-ethoxy)ethyl]piperazin-1-yl}carbonyloxymethyl)- |
| | propan-1,3-yl]-6,7,12,13-tetrahydro-5-oxo-5H-indolo[2,3-a]pyrrolo[3,4- |
| | c]carbazole MS m/z 604(M+Na), 582(M+H) |
| 167 | 12,13-(2-{[4-(benzyloxycarbonyl)piperazin-1-yl]carbonyloxymethyl}-propan-1,3- |
| | yl)-6,7,12,13-tetrahydro-5-oxo-5H-indolo[2,3-a]pyrrolo[3,4-c]carbazole MS m/z |
| | 650(M+Na), 628(M+H) |
| 168 | 12,13-(2-{[(3R)-3-hydroxy-pyrrolidin-1-yl]carbonyloxymethyl}-propan-1,3-yl)- |
| | 6,7,12,13-tetrahydro-5-oxo-5H-indolo[2,3-a]pyrrolo[3,4-c]carbazole MS m/z |
| | 517(M+Na), 495(M+H) |
| 169 | 12,13-{2-[(2-methoxy-benzyl)carbamoyloxymethyl]-propan-1,3-yl}-6,7,12,13- |
| | tetrahydro-5-oxo-5H-indolo[2,3-a]pyrrolo[3,4-c]carbazole MS m/z 545(M+H) |
| 170 | 12,13-(2-{[4-(benzo[1,3]dioxol-5-ylmethyl)piperazin-1-yl]carbonyloxymethyl}- |
| | propan-1,3-yl}-6,7,12,13-tetrahydro-5-oxo-5H-indolo[2,3-a]pyrrolo[3,4- |
| | c]carbazole MS m/z 628(M+H) |
| 171 | 12,13-(2-{[(3S)-3-dimethylamino-pyrrolidin-1-yl]carbonyloxymethyl}-propan-1,3- |
| | yl)-6,7,12,13-tetrahydro-5-oxo-5H-indolo[2,3-a]pyrrolo[3,4-c]carbazole MS m/z |
| | 522(M+H) |
| 172 | 12,13-(2-{[4-(t-butoxycarbonylamino)piperidin-1-yl]carbonyloxymethyl}-propan- |
| | 1,3-yl)-6,7,12,13-tetrahydro-5-oxo-5H-indolo[2,3-a]pyrrolo[3,4-c]carbazole MS |
| | m/z 630(M+Na), 608(M+H), 508(M+H-Boc) |
| 179 | 12,13-{3-[(2-methoxy-ethyl)carbamoyloxy]-pentan-1,5-yl}-6,7,12,13-tetrahydro-5- |
| | oxo-5H-indolo[2,3-a]pyrrolo[3,4-c]carbazole MS m/z 993(2M+H), 497(M+H) |
| 180 | 12,13-[3-(isopropyl-carbamoyloxy)-pentan-1,5-yl]-6,7,12,13-tetrahydro-5-oxo-5H- |
| | indolo[2,3-a]pyrrolo[3,4-c]carbazole MS m/z 961(2M+H), 481(M+H) |
| 181 | 12,13-(3-{[4-(4-hydroxy-phenyl)piperazin-1-yl]carbonyloxy}-pentan-1,5-yl)- |
| | 6,7,12,13-tetrahydro-5-oxo-5H-indolo[2,3-a]pyrrolo[3,4-c]carbazole MS m/z |
| | 600(M+H) |
| 182 | 12,13-[3-({2-[(1S)-2-methoxy-1-methyl]ethyl}carbamoyloxy)-pentan-1,5-yl]- |
| | 6,7,12,13-tetrahydro-5-oxo-5H-indolo[2,3-a]pyrrolo[3,4-c]carbazole MS m/z |
| | 1021(2M+H), 510(M+H) |
| 183 | 12,13-(3-{[2-(3,4-dimethoxy-phenyl)ethyl]carbamoyloxy}-pentan-1,5-yl)- |
| | 6,7,12,13-tetrahydro-5-oxo-5H-indolo[2,3-a]pyrrolo[3,4-c]carbazole MS m/z |
| | 603(M+H) |
| 184 | 12,13-{3-[(4-pyridin-4-yl-piperazin-1-yl)carbonyloxy]-pentan-1,5-yl}-6,7,12,13- |
| | tetrahydro-5-oxo-5H-indolo[2,3-a]pyrrolo[3,4-c]carbazole MS m/z 585(M+H) |
| 185 | 12,13-[3-(morpholin-4-yl-carbonyloxy)-pentan-1,5-yl]-6,7,12,13-tetrahydro-5-oxo- |
| | 5H-indolo[2,3-a]pyrrolo[3,4-c]carbazole MS m/z 1017(2M+H), 509(M+H) |
| 186 | 12,13-{3-[(3-dimethylamino-prop-1-yl)carbamoyloxy]-pentan-1,5-yl}-6,7,12,13- |
| | tetrahydro-5-oxo-5H-indolo[2,3-a]pyrrolo[3,4-c]carbazole MS m/z 523(M+H) |
| 187 | 12,13-{3-[(1-benzyl-piperidin-4-yl)carbamoyloxy]-pentan-1,5-yl}-6,7,12,13- |
| | tetrahydro-5-oxo-5H-indolo[2,3-a]pyrrolo[3,4-c]carbazole MS m/z 612(M+H) |

### Example 8

12,13-{2-[(4-methyl-piperazin-1-yl)carbonyloxy]-3-hydroxy-butan-1,4-yl}-12,13-dihydro-5,7-dioxo-6H-indolo[2,3-a]pyrrolo[3,4-c]carbazole (Compound 76)

12,13-(2,3-dihydroxy-butan-1,4-yl)-12,13-dihydro-5,7-dioxo-6H-indolo[2,3-a]pyrrolo[3,4-c]carbazole Compound 9 (505 mg, 1.228 mmol) was mixed with THF (75 mL) and CDI (1.65 g), then DMAP (1.6 g) was added. The reaction mixture was stirred at room temperature for 24 hours. The solvent was removed under vacuum and the residue was diluted with H₂O then filtered. The precipitate washed with H₂O, MeOH and CH₂Cl₂ (3× each), then dried in a vacuum oven to give a crude product as a yellow solid, which was used in the next step without further purification.

The crude product (180 mg) was mixed with methylsulfoxide (4 mL) and N-methylpiperidine (200 mL) was added. The mixture was heated to 60° C. for 3 hours. Upon cooling, the mixture was extracted with EtOAc and washed with NaCl (aq.) solution. The layers were separated and the aqueous layer was re-extracted with solution. The organic layers were combined and dried over Na₂SO₄, then concentrated and purified by reverse phase HPLC to give Compound 76 (31 mg) as a yellow solid: ¹H NMR (d-DMSO) δ 2.56 (s, 3H); 2.89 (m, 8H), 4.6 (bs, 2H), 4.71 (m, 2H), 5.02 (m, 1H), 5.26 (s, 1H), 5.84 (bs, 1H), 7.45 (t, 2H, J=7.2 Hz), 7.69 (m, 2H), 7.83 (d, 1H, J=8 Hz), 7.88 (d, 1H, J=8.4 Hz), 9.21 (t, 2H, J=10 Hz), 11.17 (s, 1H); MS m/z 538 (M+H); HRMS Calcd. for C₃₀H₂₉N₅O₅ (M+H): 538.2090. Found: 538.2090.

Using the procedure of Example 8, the following compounds were synthesized:

| Cpd | Name and Data |
|---|---|
| 69 | 12,13-(2-{[N-benzyl-N-(2-dimethylamino-ethyl)]carbamoyloxy}-3-hydroxy- |
| | butan-1,4-yl)-12,13-dihydro-5,7-dioxo-6H-indolo[2,3-a]pyrrolo[3,4-c]carbazole |
| | MS m/z 600(M+H) |
| 77 | 12,13-[2-[(2-dimethylamino-ethyl)carbamoyloxy]-3-hydroxy-butan-1,4-yl]-12,13- |
| | dihydro-5,7-dioxo-6H-indolo[2,3-a]pyrrolo[3,4-c]carbazole MS m/z 526(M+H) |
| 78 | 12,13-{2-[(2-methoxy-benzyl)carbamoyloxy]-3-hydroxy-butan-1,4-yl}-12,13- |
| | dihydro-5,7-dioxo-6H-indolo[2,3-a]pyrrolo[3,4-c]carbazole MS m/z |
| | 597(M+Na), 575(M+H) |
| 79 | 12,13-(2-{[3-(2-oxo-pyrrolidin-1-yl)prop-1-yl]carbamoyloxy}-3-hydroxy-butan- |
| | 1,4-yl)-12,13-dihydro-5,7-dioxo-6H-indolo[2,3-a]pyrrolo[3,4-c]carbazole MS |
| | m/z 602(M+Na), 580(M+H) |
| 80 | 12,13-{2-[(benzo[1,3]dioxol-5-yl-methyl)carbamoyloxy]-3-hydroxy-butan-1,4- |
| | yl}-12,13-dihydro-5,7-dioxo-6H-indolo[2,3-a]pyrrolo[3,4-c]carbazole MS m/z |
| | 612(M+Na), 589(M+H) |
| 81 | 12,13-{2-[(cyclohexyl-methyl)carbamoyloxy]-3-hydroxy-butan-1,4-yl}-12,13- |
| | dihydro-5,7-dioxo-6H-indolo[2,3-a]pyrrolo[3,4-c]carbazole MS m/z |
| | 573(M+Na), 551(M+H) |
| 82 | 12,13-{2-[(2-pyridin-2-yl-ethyl)carbamoyloxy]-3-hydroxy-butan-1,4-yl}-12,13- |
| | dihydro-5,7-dioxo-6H-indolo[2,3-a]pyrrolo[3,4-c]carbozole MS m/z 560(M+H) |
| 83 | 12,13-{2-[(2-methoxy-ethyl)carbamoyloxy]-3-hydroxy-butan-1,4-yl}-12,13- |
| | dihydro-5,7-dioxo-6H-indolo[2,3-a]pyrrolo[3,4-c]carbazole MS m/z |
| | 535(M+Na), 513(M+H) |
| 84 | 12,13-(2-{[2-(3,4-dimethoxy-phenyl)ethyl]carbamoyloxy}-3-hydroxy-butan-1,4- |
| | yl)-12,13-dihydro-5,7-dioxo-6H-indolo[2,3-a]pyrrolo[3,4-c]carbazole MS m/z |
| | 641(M+Na), 619(M+H) |
| 85 | 12,13-[2-(prop-2-yl-carbamoyloxy)-3-hydroxy-butan-1,4-yl]-12,13-dihydro-5,7- |
| | dioxo-6H-indolo[2,3-a]pyrrolo[3,4-c]carbazole MS m/z 519(M+Na), 497(M+H) |
| 86 | 12,13-(2-{[(5-methyl-furan-2-yl)methyl]carbamoyloxy}-3-hydroxy-butan-1,4-yl)- |
| | 12,13-dihydro-5,7-dioxo-6H-indolo[2,3-a]pyrrolo[3,4-c]carbazole MS m/z |
| | 549(M+H) |
| 87 | 12,13-(2-{[2-(5-methoxy-1H-indol-3-yl)ethyl]carbamoyloxy}-3-hydroxy-butan- |
| | 1,4-yl)-12,13-dihydro-5,7-dioxo-6H-indolo[2,3-a]pyrrolo[3,4-c]carbazole MS |
| | m/z 650(M+Na), 628(M+H) |
| 88 | 12,13-{2-[(3-morpholin-4-yl-prop-1-yl)carbamoyloxy]-3-hydroxy-butan-1,4-yl}- |
| | 12,13-dihydro-5,7-dioxo-6H-indolo[2,3-a]pyrrolo[3,4-c]carbazole MS m/z |
| | 582(M+H) |
| 89 | 12,13-{2-[(pyridin-4-yl-methyl)carbamoyloxy]-3-hydroxy-butan-1,4-yl}-12,13- |
| | dihydro-5,7-dioxo-6H-indolo[2,3-a]pyrrolo[3,4-c]carbazole MS m/z |
| | 575(M+Na), 546(M+H) |
| 90 | 12,13-(2-{[3-(4-methyl-piperazin-1-yl)prop-1-yl]carbamoyloxy}-3-hydroxy- |
| | butan-1,4-yl)-12,13-dihydro-5,7-dioxo-6H-indolo[2,3-a]pyrrolo[3,4-c]carbazole |
| | MS m/z 595(M+H) |
| 91 | 12,13-{2-[(3-imidazol-1-yl-prop-1-yl)carbamoyloxy]-3-hydroxy-butan-1,4-yl}- |
| | 12,13-dihydro-5,7-dioxo-6H-indolo[2,3-a]pyrrolo[3,4-c]carbazole MS m/z |
| | 585(M+Na), 563(M+H) |
| 92 | 12,13-{2-[(2-pyrrolidin-1-yl-ethyl)carbamoyloxy]-3-hydroxy-butan-1,4-yl}- |
| | 12,13-dihydro-5,7-dioxo-6H-indolo[2,3-a]pyrrolo[3,4-c]carbazole MS m/z |
| | 574(M+Na), 552(M+H) |
| 93 | 12,13-{2-[(4-dimethylamino-benzyl)carbamoyloxy]-3-hydroxy-butan-1,4-yl}- |
| | 12,13-dihydro-5,7-dioxo-6H-indolo[2,3-a]pyrrolo[3,4-c]carbazole MS m/z |
| | 610(M+Na), 588(M+H) |
| 94 | 12,13-(2-{[(4-(2-morpholin-4-yl-ethyl)piperazin-1-yl]carbonyloxy}-3-hydroxy- |
| | butan-1,4-yl)-12,13-dihydro-5,7-dioxo-6H-indolo[2,3-a]pyrrolo[3,4-c]carbazole |
| | MS m/z 637(M+H) |
| 95 | 12,13-[2-({4-[(pyrrolidin-1-yl-methyl)carbonyl]piperazin-1-yl}carbonyloxy)-3- |
| | hydroxy-butan-1,4-yl]-12,13-dihydro-5,7-dioxo-6H-indolo[2,3-a]pyrrolo[3,4- |
| | c]carbazole MS m/z 657(M+Na), 635(M+H) |
| 96 | 12,13-{2-[(4-pyridin-4-yl-piperazin-1-yl)carbonyloxy]-3-hydroxy-butan-1,4-yl}- |
| | 12,13-dihydro-5,7-dioxo-6H-indolo[2,3-a]pyrrolo[3,4-c]carbazole MS m/z |
| | 601(M+H) |
| 97 | 12,13-(2-{[4-(3-dimethylamino-prop-1-yl)piperazin-1-yl]carbonyloxy}-3- |
| | hydroxy-butan-1,4-yl)-12,13-dihydro-5,7-dioxo-6H-indolo[2,3-a]pyrrolo[3,4- |
| | c]carbazole MS m/z 609(M+H) |
| 98 | 12,13-[2-(morpholin-4-yl-carbonyloxy)-3-hydroxy-butan-1,4-yl]-12,13-dihydro- |
| | 5,7-dioxo-6H-indolo[2,3-a]pyrrolo[3,4-c]carbazole MS m/z 1071(2M+Na), |
| | 547(M+Na), 525(M+H) |
| 99 | 12,13-[2-(piperidin-1-yl-carbonyloxy)-3-hydroxy-butan-1,4-yl]-12,13-dihydro- |
| | 5,7-dioxo-6H-indolo[2,3-a]pyrrolo[3,4-c]carbazole MS m/z 1067(2M+Na), |
| | 545(M+Na), 523(M+H) |
| 100 | 12,13-{2-[(3-dimethyl-pyrrolidin-1-yl)carbonyloxy]-3-hydroxy-butan-1,4-yl}- |
| | 12,13-dihydro-5,7-dioxo-6H-indolo[2,3-a]pyrrolo[3,4-c]carbazole MS m/z |
| | 552(M+H) |
| 101 | 12,13-{2-[(4-cyclohexyl-piperazin-1-yl)carbonyloxy]-3-hydroxy-butan-1,4-yl}- |
| | 12,13-dihydro-5,7-dioxo-6H-indolo[2,3-a]pyrrolo[3,4-c]carbazole MS m/z |
| | 606(M+H) |
| 102 | 12,13-{2-[(4-phenyl-piperazin-1-yl)carbonyloxy]-3-hydroxy-butan-1,4-yl}-12,13- |
| | dihydro-5,7-dioxo-6H-indolo[2,3-a]pyrrolo[3,4-c]carbazole MS m/z |
| | 622(M+Na), 600(M+H) |
| 103 | 12,13-{2-[(4-benzhydryl-piperazin-1-yl)carbonyloxy]-3-hydroxy-butan-1,4-yl}- |
| | 12,13-dihydro-5,7-dioxo-6H-indolo[2,3-a]pyrrolo[3,4-c]carbazole MS m/z |
| | 690(M+H) |
| 104 | 12,13-(2-{[4-(2-hydroxy-ethyl)piperazin-1-yl]carbonyloxy}-3-hydroxy-butan-1,4- |
| | yl)-12,13-dihydro-5,7-dioxo-6H-indolo[2,3-a]pyrrolo[3,4-c]carbazole MS m/z |
| | 590(M+Na), 568(M+H) |
| 105 | 12,13-(2-{[2-(4-sulfonylamino-phenyl)ethyl]carbamoyloxy}-3-hydroxy-butan- |
| | 1,4-yl)-12,13-dihydro-5,7-dioxo-6H-indolo[2,3-a]pyrrolo[3,4-c]carbazole MS |
| | m/z 660(M+Na), 638(M+H) |
| 106 | 12,13-{2-[(1-benzyl-piperidin-4-yl)carbamoyloxy]-3-hydroxy-butan-1,4-yl}- |
| | 12,13-dihydro-5,7-dioxo-6H-indolo[2,3-a]pyrrolo[3,4-c]carbazole MS m/z |
| | 628(M+H) |
| 107 | 12,13-(2-{[N-methyl-N-(2-dimethylamino-ethyl)]carbamoyloxy}-3-hydroxy- |
| | butan-1,4-yl)-12,13-dihydro-5,7-dioxo-6H-indolo[2,3-a]pyrrolo[3,4-c]carbazole |
| | MS m/z 540(M+H) |
| 108 | 12,13-(2-{[N-methyl-N-(1-methyl-pyrrolidin-3-yl)]carbamoyloxy}-3-hydroxy- |
| | butan-1,4-yl)-12,13-dihydro-5,7-dioxo-6H-indolo[2,3-a]pyrrolo[3,4-c]carbazole |
| | MS m/z 574(M+Na), 552(M+H) |
| 109 | 12,13-(2-{[N,N-bis-(3-dimethylamino-prop-1-yl)]carbamoyloxy}-3-hydroxy- |
| | butan-1,4-yl)-12,13-dihydro-5,7-dioxo-6H-indolo[2,3-a]pyrrolo[3,4-c]carbazole |
| | MS m/z 625(M+H) |
| 110 | 12,13-(2-{[4-(2-phenyl-ethyl)piperazin-1-yl]carbamoyloxy}-3-hydroxy-butan-1,4- |
| | yl)-12,13-dihydro-5,7-dioxo-6H-indolo[2,3-a]pyrrolo[3,4-c]carbazole MS m/z |
| | 628(M+H) |
| 111 | 12,13-{2-[(hexahydro-1H-1,4-diazepin-1-yl)carbonyloxy]-3-hydroxy-butan-1,4- |
| | yl}-12,13-dihydro-5,7-dioxo-6H-indolo[2,3-a]pyrrolo[3,4-c]carbazole MS m/z |
| | 538(M+H) |
| 112 | 12,13-{2-[(4-pyridin-2-yl-piperazin-1-yl)carbonyloxy]-3-hydroxy-butan-1,4-yl}- |
| | 12,13-dihydro-5,7-dioxo-6H-indolo[2,3-a]pyrrolo[3,4-c]carbazole MS m/z |
| | 601(M+H) |
| 113 | 12,13-[2-(piperidin-4-yl-carbamoyloxy)-3-hydroxy-butan-1,4-yl]-12,13-dihydro- |
| | 5,7-dioxo-6H-indolo[2,3-a]pyrrolo[3,4-c]carbazole MS m/z 538(M+H) |
| 135 | 12,13-[2-hydroxy-2-(isopropyl-carbamoyloxymethyl)-propan-1,3-yl]-6,7,12,13- |
| | tetrahydro-5-oxo-5H-indolo[2,3-a]pyrrolo[3,4-c]carbazole MS m/z 505(M+Na), |
| | 483(M+H) |
| 136 | 12,13-{2-hydroxy-2-[(2-methoxy-ethyl)carbamoyloxymethyl]-propan-1,3-yl}- |
| | 6,7,12,13-tetrahydro-5-oxo-5H-indolo[2,3-a]pyrrolo[3,4-c]carbazole MS m/z |
| | 521(M+Na), 499(M+H) |
| 137 | 12,13-(2-hydroxy-2-{[4-(4-hydroxy-phenyl)piperazin-1-yl]carbonyloxymethyl}- |
| | propan-1,3-yl)-6,7,12,13-tetrahydro-5-oxo-5H-indolo[2,3-a]pyrrolo[3,4- |
| | c]carbazole MS m/z 624(M+Na), 602(M+H) |
| 138 | 12,13-[2-hydroxy-2-(morpholin-4-yl-carbonyloxymethyl)-propan-1,3-yl]- |
| | 6,7,12,13-tetrahydro-5-oxo-5H-indolo[2,3-a]pyrrolo[3,4-c]carbazole MS m/z |
| | 533(M+Na), 511(M+H) |
| 139 | 12,13-{2-hydroxy-2-[(2-pyridin-2-yl-ethyl)carbamoyloxymethyl)]-propan-1,3-yl}- |
| | 6,7,12,13-tetrahydro-5-oxo-5H-indolo[2,3-a]pyrrolo[3,4-c]carbazole MS m/z |
| | 568(M+Na), 546(M+H) |
| 140 | 12,13-(2-hydroxy-2-{[3-(2-oxo-pyrrolidin-1-yl)-prop-1-yl]carbamoyloxymethyl}- |
| | propan-1,3-yl)-6,7,12,13-tetrahydro-5-oxo-5H-indolo[2,3-a]pyrrolo[3,4- |
| | c]carbazole MS m/z 588(M+Na), 566(M+H) |
| 141 | 12,13-(2-hydroxy-2-{[(5-methyl-furan-2-yl)methyl]carbamoyloxymethyl}- |
| | propan-1,3-yl)-6,7,12,13h-tetrahydro-5-oxo-5H-indolo[2,3-a]pyrrolo[3,4- |
| | c]carbazole MS m/z 557(M+Na), 535(M+H) |
| 142 | 12,13-{2-hydroxy-2-[(4-cyclohexyl-piperazin-1-yl)carbonyloxymethyl]-propan- |
| | 1,3-yl}-6,7,12,13-tetrahydro-5-oxo-5H-indolo[2,3-a]pyrrolo[3,4-c]carbazole MS |
| | m/z 592(M+H) |
| 143 | 12,13-{2-hydroxy-2-[(4-hydroxymethyl-piperidin-1-yl)carbonyloxymethyl]- |
| | propan-1,3-yl}-6,7,12,13-tetrahydro-5-oxo-5H-indolo[2,3-a]pyrrolo[3,4- |
| | c]carbazole MS m/z 539(M+H) |
| 144 | 12,13-{2-hydroxy-2-[(4-pyridin-4-yl-piperazin-1-yl)carbonyloxymethyl]-propan- |
| | 1,3-yl}-6,7,12,13-tetrahydro-5-oxo-5H-indolo[2,3-a]pyrrolo[3,4-c]carbazole MS |
| | m/z 587(M+H) |
| 145 | 12,13-{2-hydroxy-2-[(1-benzyl-piperidin-4-yl)carbamoyloxymethyl]-propan-1,3- |
| | yl}-6,7,12,13-tetrahydro-5-oxo-5H-indolo[2,3-a]pyrrolo[3,4-c]carbazole MS m/z |
| | 614(M+H) |
| 146 | 12,13-{2-hydroxy-2-[(hexahydro-1H-1,4-diazepin-1-yl)carbonyloxymethyl]- |
| | propan-1,3-yl}-6,7,12,13-tetrahydro-5-oxo-5H-indolo[2,3-a]pyrrolo[3,4- |
| | c]carbazole MS m/z 524(M+H) |
| 147 | 12,13-{2-hydroxy-2-[(1,2,3,4-tetrahydro-isoquinolin-2-yl)carbonyloxymethyl]- |
| | propan-1,3-yl}-6,7,12,13-tetrahydro-5-oxo-5H-indolo[2,3-a]pyrrolo[3,4- |
| | c]carbazole MS m/z 579(M+Na), 557(M+H) |
| 148 | 12,13-(2-hydroxy-2-{[N-methyl-N-(2-dimethylamino- |
| | ethyl)]carbamoyloxymethyl}-propan-1,3-yl)-6,7,12,13-tetrahydro-5-oxo-5H- |
| | indolo[2,3-a]pyrrolo[3,4-c]carbazole MS m/z 548(M+Na), 426(M+H) |
| 149 | 12,13-{2-hydroxy-2-[(4-hydroxy-piperidin-1-yl)carbonyloxymethyl]-propan-1,3- |
| | yl}-6,7,12,13-tetrahydro-5-oxo-5H-indolo[2,3-a]pyrrolo[3,4-c]carbazole MS m/z |
| | 547(M+Na), 525(M+H) |
| 150 | 12,13-{2-hydroxy-2-[(4-pyrrolidin-1-yl-piperidin-1-yl)carbonyloxymethyl]- |
| | propan-1,3-yl}-6,7,12,13-tetrahydro-5-oxo-5H-indolo[2,3-a]pyrrolo[3,4- |
| | c]carbazole MS m/z 578(M+H) |
| 151 | 12,13-(2-hydroxy-2-{[3-(4-methyl-piperazin-1-yl)prop-1- |
| | yl]carbamoyloxymethyl}-propan-1,3-yl)-6,7,12,13-tetrahydro-5-oxo-5H- |
| | indolo[2,3-a]pyrrolo[3,4-c]carbazole MS m/z 581(M+H) |
| 152 | 12,13-{2-hydroxy-2-[(3-1H-imidazol-1-yl-prop-1-yl)carbamoyloxymethyl]- |
| | propan-1,3-yl}-6,7,12,13-tetrahydro-5-oxo-5H-indolo[2,3-a]pyrrolo[3,4- |
| | c]carbazole MS m/z 549(M+H) |
| 153 | 12,13-(2-hydroxy-2-{[2-(3,4-dimethoxy-phenyl)ethyl]carbamoyloxymethyl}- |
| | propan-1,3-yl)-6,7,12,13-tetrahydro-5-oxo-5H-indolo[2,3-a]pyrrolo[3,4- |
| | c]carbazole MS m/z 627(M+Na), 605(M+H) |
| 173 | 12,13-{2-hydroxy-2-[(2-dimethylamino-ethyl)carbamoyloxymethyl]-propan-1,3- |
| | yl}-6,7,12,13-tetrahydro-5-oxo-5H-indolo[2,3-a]pyrrolo[3,4-c]carbazole MS m/z |
| | 512(M+H) |
| 174 | 12,13-{2-hydroxy-2-[(2-methoxy-benzyl)carbamoyloxymethyl]-propan-1,3-yl}- |
| | 6,7,12,13-tetrahydro-5-oxo-5H-indolo[2,3-a]pyrrolo[3,4-c]carbazole MS m/z |
| | 561(M+H) |
| 175 | 12,13-{2-hydroxy-2-[(4-t-butoxyamido-piperidin-1-yl)carbonyloxymethyl]- |
| | propan-1,3-yl}-6,7,12,13-tetrahydro-5-oxo-5H-indolo[2,3-a]pyrrolo[3,4- |
| | c]carbazole MS m/z 624(M+H), 524(M+H-Boc) |
| 176 | 12,13-{2-hydroxy-2-[(4-amino-piperidin-1-yl)carbonyloxymethyl]-propan-1,3- |
| | yl}-6,7,12,13-tetrahydro-5-oxo-5H-indolo[2,3-a]pyrrolo[3,4-c]carbazole MS m/z |
| | 524(M+H) |
| 177 | 12,13-{2-hydroxy-2-[(1-t-butoxycarbonyl-piperidin-4-yl)carbamoyloxymethyl]- |
| | propan-1,3-yl}-6,7,12,13-tetrahydro-5-oxo-5H-indolo[2,3-a]pyrrolo[3,4- |
| | c]carbazole MS m/z 646(M+Na), 524(M+H-Boc) |
| 178 | 12,13-[2-hydroxy-2-(piperidin-4-yl-carbamoyloxymethyl)-propan-1,3-yl]- |
| | 6,7,12,13-tetrahydro-5-oxo-5H-indolo[2,3-a]pyrrolo[3,4-c]carbazole MS m/z |
| | 524(M+H) |

### Example 9

12,13-[(4R,5R)-2,2-dimethyl-[1,3]dioxolo[4,5-b]butan-1,4-yl]-6,7,12,13-tetrahydro-5-oxo-5H-indolo[2,3-a]pyrrolo[3,4-c]carbazole (Compound 17)
12,13-[(2R,3R)-dihydroxy-butan-1,4-yl]-6,7,12,13-tetrahydro-5-oxo-5H-indolo[2,3-a]pyrrolo[3,4-c]carbazole (Compound 19)

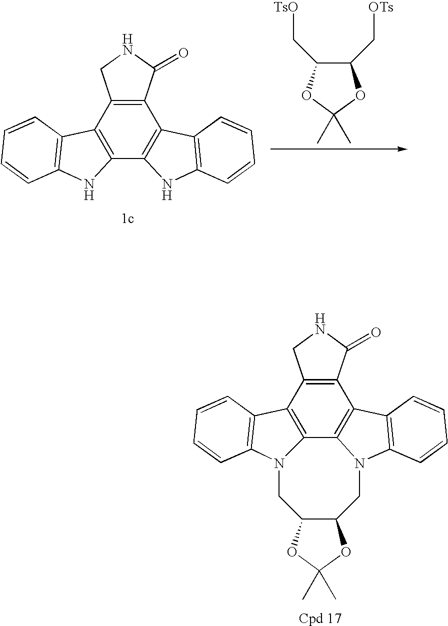

6,7,12,13-tetrahydro-5-oxo-5H-indolo[2,3-a]pyrrolo[3,4-c]carbazole Compound 1c (65 mg), Cs₂CO₃ (315 mg), 4,5-bis-[(2-methyl-phenyl)sulfonyloxymethyl]-2,2-dimethyl-[1,3]dioxolane (122 mg) and acetonitrile (4 mL) were added to a microwave tube. The tube was placed inside a microwave instrument and irradiated for 3400 seconds at 150° C. The process was repeated seven more times for additional batches of Compound 1c (8×65 mg=520 mg total). The contents of all eight vessels were combined, extracted with ethyl acetate and sequentially washed with solutions of NH₄Cl (aq.) and NaCl (aq.). The organic layers were separated, the solvent was removed under vacuum and the resulting residue was purified via column chromatography (ethyl acetate/hexanes gradiant) to provide Compound 17 (267 mg) as a light orange solid (37% yield), after fraction combination and solvent removal. ¹H NMR (300 MHz, d⁶-DMSO): δ 1.46 (s, 3H), 1.48 (s, 3H), 4.56 (s, 2H), 4.68 (m, 4H), 5.0 (s, 2H), 7.34 (t, 1H, J=8 Hz), 7.42 (t, 1H, J=7 Hz), 7.58 (quint, 2H, J=7 Hz), 7.74 (d, 1H, J=8 Hz), 7.83 (d, 1H, J=8 Hz), 8.08 (d, 1H, J=9 Hz), 8.68 (s, 1H), 9.47 (d, 1H, J=8 Hz); MS m/z 897 (2M+Na), 875 (2M+H), 438 (M+H).

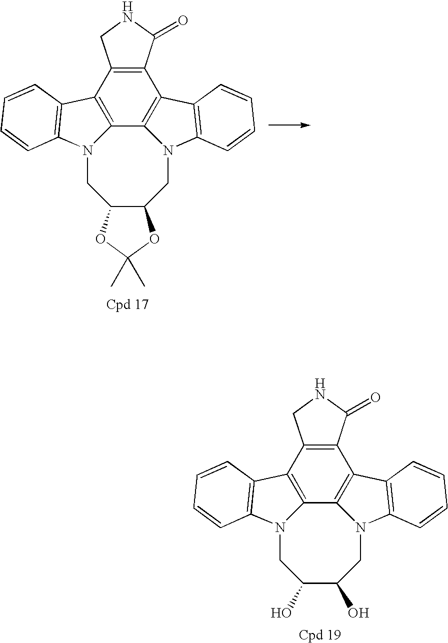

p-toluenesulfonic acid (5 mg) and water (5 drops) were added to a solution of Compound 17 (45 mg) in THF (2 mL) and methanol (1 mL). The mixture was heated to 75° C. for 3 hours, then cooled. The solids were filtered, washed three times each with methanol, ethyl acetate, water, methanol and DCM, then dried to provide Compound 19 (34 mg, 83% yield) as a light gray solid. ¹H NMR (300 MHz, d⁶-DMSO): δ 3.99 (m, 2H), 4.72 (m, 4H), 4.97 (s, 2H), 5.47 (s, 2H), 7.28 (t, 1H, J=8 Hz), 7.37 (t, 1H, J=7 Hz), 7.54 (quint, 2H, J=8 Hz), 7.74 (d, 1H, J=8 Hz), 7.82 (d, 1H, J=8 Hz), 8.05 (d, 1H, J=8 Hz), 8.57 (s, 1H), 9.44 (d, 1H, J=8 Hz); MS m/z 795 (2M+H), 398 (M+H).

Using the procedure of Example 9, the following compounds were synthesized:

| Cpd | Name and Data |
|---|---|
| 16 | 12,13-[(4S,5S)-2,2-dimethyl-[1,3]dioxolo[4,5-b]butan-1,4-yl]-6,7,12,13- |
| | tetrahydro-5-oxo-5H-indolo[2,3-a]pyrrolo[3,4-c]carbazole |
| | ¹H NMR(300MHz, CDCl₃):δ 1.44(s, 3H), 1.45(s, 3H), 4.55(s, 2H), 4.59(m, |
| | 1H), 4.68(m, 3H), 4.98(s, 2H), 7.32(t, 1H, J=8Hz), 7.4(t, 1H, J=8Hz), |
| | 7.56(quint, 2H, J=7Hz), 7.73(d, 1H, J=8Hz), 7.81(d, 1H, J=8Hz), 8.06(d, 1H, J=8Hz), |
| | 8.66(s, 1H), 9.45(d, 1H, J=8Hz); MS: 897(2M+Na), 875(2M+H), |
| | 438(M+H). |
| 18 | 12,13-[(2S,3S)-dihydroxy-butan-1,4-yl]-6,7,12,13-tetrahydro-5-oxo-5H- |
| | indolo[2,3-a]pyrrolo[3,4-c]carbazole |
| | ¹H NMR(300MHz, d⁶-DMSO):δ 3.99(m, 2H), 4.72(m, 4H), 4.97(s, 2H), |
| | 5.47(s, 2H), 7.28(t, 1H, J=8Hz), 7.37(t, 1H, J=7Hz), 7.54(quint, 2H, J=8Hz), |
| | 7.74(d, 1H, J=8Hz), 7.82(d, 1H, J=8Hz), 8.05(d, 1H, J=8Hz), 8.57(s, 1H), 9.44(d, |
| | 1H, J=8Hz); MS: 795(2M+H), 398(M+H) |
| 24 | 12,13-(2-{[1,1-bis-(dihydroxymethyl)]methylidene}-propan-1,3-yl)-6,7,12,13- |
| | tetrahydro-5-oxo-5H-indolo[2,3-a]pyrrolo[3,4-c]carbazole |
| | ¹H NMR: (d⁶-DMSO)δ 4.16(s, 2H), 4.82(s, 2H), 4.93(s, 2H), 5.37(s, 2H), |
| | 5.40(s, 2H), 7.37(t, 1H, J=7.8Hz), 7.56-7.65(m, 2H), 7.85(d, 1H, J=9.0Hz), |
| | 7.94(d, 1H, J=8.4Hz), 8.06(d, 1H, J=7.5Hz), 8.59(s, 1H), 9.47(d, 1H, |
| | J=1.8Hz) |
| 26 | 12,13-{(2-[(5-spiro)-(2,2-dimethyl-[1,3]dioxan-5-yl)]-propan-1,3-yl}-6,7,12,13- |
| | tetrahydro-5-oxo-5H-indolo[2,3-a]pyrrolo[3,4-c]carbazole |
| | ¹H NMR: (d⁶-DMSO)δ 1.44(s, 6H), 3.83(s, 4H), 4.61(s, 2H), 4.66(s, 2H), |
| | 4.88(s, 2H), 7.29(t, 1H, J=7.2Hz), 7.37(t, 1H, J=7.2Hz), 7.50-7.60(m, 2H), |
| | 7.71(d, 1H, J=7.8Hz), 7.77(d, 1H, J=7.5Hz), 8.04(d, 1H, J=6.3Hz), |
| | 8.51(s, 1H), 9.31(d, 1H, J=7.5Hz). MS m/z 474(M+Na), 452(M+H) |
| 27 | 12,13-(2,2-bis-hydroxymethyl-propan-1,3-yl)-6,7,12,13-tetrahydro-5-oxo-5H- |
| | indolo[2,3-a]pyrrolo[3,4-c]carbazole |
| | ¹H NMR: (d⁶-DMSO)δ 3.59(d, 4H, J=4.8Hz), 4.59(s, 4H), 4.90(t, 2H, J=4.8Hz), |
| | 4.96(s, 2H), 7.28(t, 1H, J=7.5Hz), 7.36(t, 1H, J=7.5Hz), 7.51(t, 1H, |
| | J=8.1Hz), 7.55(t, 1H, J=8.1Hz), 7.68(d, 1H, J=8.4Hz), 7.76(d, 1H, J=8.1Hz), |
| | 8.07(d, 1H, J=7.8Hz), 8.52(s, 1H), 9.32(d, 1H, J=7.8Hz). MS |
| | m/z 434(M+Na), 412(M+H). |
| 28 | 12,13-(3-hydroxy-pentan-1,5-yl)-6,7,12,13-tetrahydro-5-oxo-5H-indolo[2,3- |
| | a]pyrrolo[3,4-c]carbazole |
| | ¹H NMR(d⁶-DMSO, 300MHz)δ 2.07-2.26(m, 4H), 4.96(s, 2H), 5.05-5.18(m, |
| | 5H), 7.27(t, 1H, J=8Hz), 7.37(t, 1H, J=8Hz), 7.54(quintet, 2H, J=8Hz), |
| | 7.76(d, 1H, J=8Hz), 7.86(d, 1H, J=8Hz), 8.04(d, 1H, J=8Hz), 9.61(d, 1H, J=8Hz); |
| | MS m/z 791(2M+H), 396(M+H) |
| 29 | 12,13-(3-oxa-pentan-1,5-yl)-6,7,12,13-tetrahydro-5-oxo-5H-indolo[2,3- |
| | a]pyrrolo[3,4-c]carbazole |
| | ¹H NMR: (d⁶-DMSO)δ 4.04 br s, 4H), 5.00(s, 2H), 5.0-5.5(br s, 4H), 7.27(t, 1H, |
| | J=7.5Hz), 7.38(t, 1H, J=7.5Hz), 7.50(t, 1H, J=7.2Hz), 7.56(t, 1H, J=7.5Hz), |
| | 7.80(d, 1H, J=8.4Hz), 7.89(d, 1H, J=8.4Hz), 8.07(d, 1H, J=7.5Hz), |
| | 8.59(s, 1H), 9.66(d, 1H, J=7.5Hz). MS m/z 404(M+Na), 382(M+H) |
| 30 | 12,13-(1H-pyrrol[3,4-b]butan-1,4-yl)-6,7,12,13-tetrahydro-5-oxo-5H-indolo[2,3- |
| | a]pyrrolo[3,4-c]carbazole |
| | ¹H NMR:(d⁶-DMSO)δ 4.95(s, 2H), 5.73(s, 2H), 5.75(s, 2H), 6.88(s, 2H), |
| | 6.89(s, 2H), 7.27(t, 1H, J=7.5Hz), 7.35(t, 1H, J=7.5Hz), 7.54(t, 1H, J=7.5Hz), |
| | 7.59(t, 1H, J=7.5Hz), 8.03(d, 1H, J=7.5Hz), 8.04(d, 1H, J=8.4Hz), |
| | 8.14(d, 1H, J=8.4Hz), 8.54(s, 1H), 9.61(d, 1H, J=7.8Hz), 10.08(s, 1H). |
| | MS m/z 403(M+H) |

### Example 10

3-pyridin-3-yl-12,13-(2-hydroxy-butan-1,4-yl)-12,13-dihydro-5,7-dioxo-6H-indolo[2,3-a]pyrrolo[3,4-c]carbazole (Compound 32)

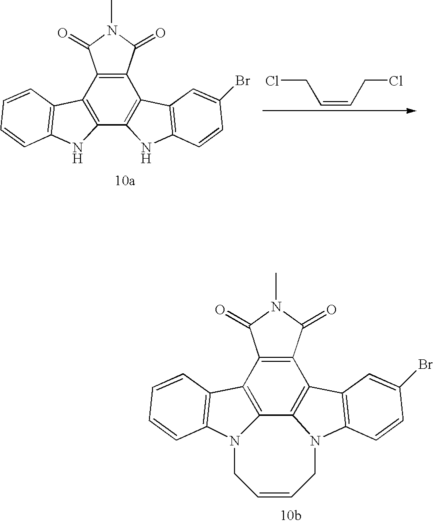

3-bromo-6-methyl-12,13-dihydro-5,7-dioxo-6H-indolo[2,3-a]pyrrolo[3,4-c]carbazole Compound 10a (2.37 g, 7 mmol) (prepared as described in Slater M J, Bioorganic & Medicinal Chemistry, 1999, 7, 1067) was dissolved in DMF (20 mL) and Cs₂CO₃ (1.95 g, 6.0 mmol) and 1,4 dichloro-but-2-ene (310 μL, 3.0 mmol) were added. The reaction mixture was stirred at 60° C. for 18 hrs and quenched with water (200 mL). The precipitate was collected by filtration, rinsed with water and dried in a vacuum oven to provide 3-bromo-6-methyl-12,13-(but-2-en-1,4-yl)-12,13-dihydro-5,7-dioxo-6H-indolo[2,3-a]pyrrolo[3,4-c]carbazole Compound 10b (510 mg, 91%) as a yellow solid. ¹H NMR (300 MHz, d₆-DMSO) δ 3.16 (s, 3H), 5.46 (d, 4H, J=5 Hz), 6.51 (m, 2H), 7.43 (t, 1H, J=8 Hz), 7.68 (t, 1H, J=2 Hz), 7.80 (d, 1H, J=2 Hz), 8.97 (dd, 2H, J=4, 5 Hz), 9.32 (d, 1H, J=8 Hz), 9.50 (d, 1H, J=2 Hz).

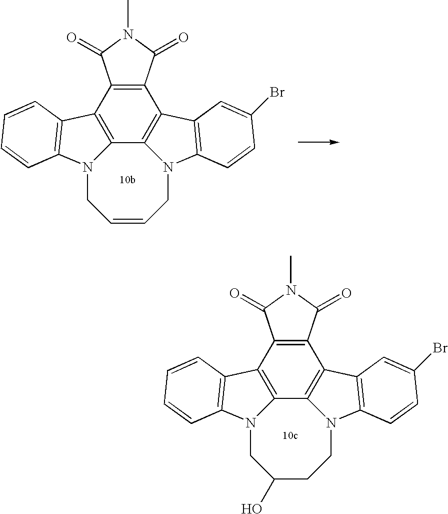

Compound 10b (500 mg, 1.06 mol) in THF (50 mL) was added to a borane-THF complex (1M, 5.3 mL, 5.32 mmol) at room temperature. The reaction mixture was stirred at room temperature for 3 hrs until the color turned to a yellow homogeneous solution. The mixture was cooled in an ice bath, then aqueous H₂O₂ solution (50%, 25 mL) was added slowly over a period of 15 minutes, followed by addition of aqueous 2N NaOH solution (75 mL) over a period of 40 minutes. The mixture was then diluted with water (50 mL) and extracted with ethyl acetate. The organic layer washed with brine and dried over Na₂SO₄, then concentrated to provide 3-bromo-6-methyl-12,13-(2-hydroxy-butan-1,4-yl)-12,13-dihydro-5,7-dioxo-6H -indolo[2,3-a]pyrrolo[3,4-c]carbazole Compound 10c (510 mg, 98%) as a yellow solid. ¹H NMR (300 MHz, d₆-DMSO) δ 3.16 (s, 3H), 4.53 (m, 4H), 5.38 (b, 1H), 7.41 (m, 1H), 7.67 (m, 1H), 7.80 (m, 3H), 9.12 (dd, 1H, J=8, 8 Hz), 9.32 (d, 1H, J=2, 10 Hz).

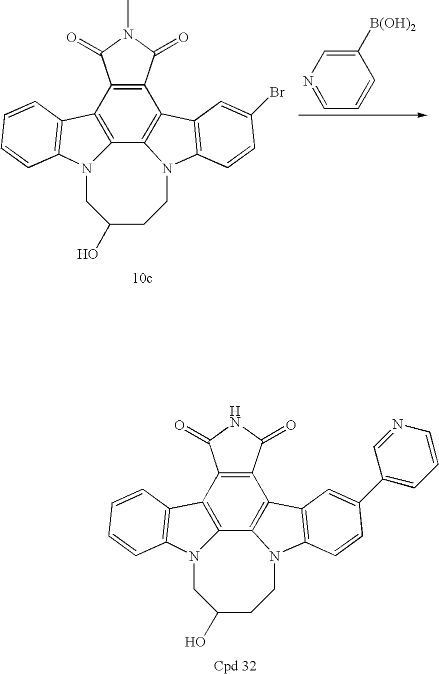

Compound 10c (60 mg, 0.122 mmol) was suspended in DMF (3 mL) and 3-pyridine boronic acid (45 mg), tetrakis(triphenylphosphine)palladium (2 mg) and 2M Na₂CO₃ (0.3 mL) were added. The mixture was irradiated in a sealed vessel in a microwave oven at 150° C. for 20 min, then the solvent was removed under vacuum. The resulting residue was stirred with water and filtered, then the solid washed with methanol and dried under vacuum. The solids were then combined with 1 pellet of KOH and ethanol (2.5 mL) in a sealed microwave vessel and irradiated in a microwave instrument at 150° C. for 20 min. The mixture was cooled to room temperature and filtered through silica cartridge with ethanol to remove the palladium metal. The ethanol was partially removed and the residue was acidified with 1N HCl to pH 1. The resulting precipitate was, collected by filtration to give a yellow solid. The solid was heated with ammonium acetate (200 mg) in a sealed microwave vessel and irradiated in a microwave instrument at 180° C. for 20 min. The mixture was cooled and water was added. The resulting precipitate was collected by filtration and washed with excess methanol to give Compound 32 (19 mg, 68%) as a yellow solid. ¹H NMR (300 MHz, d₆-DMSO) δ 4.53 (m, 5H), 5.45 (m, 1H), 7.39 (m, 1H), 7.55 (m, 1H), 7.66 (m, 1H), 7.87 (m, 1H), 7.96 (m, 2H), 8.18 (m, 1H), 8.60 (d, 1H, J=5 Hz), 9.0 (m, 1H), 9.16 (dd, 1H, J=8, 10 Hz), 9.58 (m, 1H), 11.18 (s, 1H); MS m/z 473 (M+H)

Using the procedure of Example 10, the following compounds were synthesized:

| Cpd | Name and Data |
|---|---|
| 31 | 3-bromo-12,13-(2-hydroxy-butan-1,4-yl)-12,13-dihydro-5,7-dioxo-6H-indolo[2,3- |
| | a]pyrrolo[3,4-c]carbazole |
| | ¹H NMR(300MHz, d⁶-DMSO)δ 4.53(m, 4H), 5.42(t, 1H, J=4Hz), 7.43(m, |
| | 1H), 7.68(m, 1H), 7.80(m, 3H), 9.12(dd, 1H, J=8.8Hz), 9.32(d, 1H, J=2.10Hz), |
| | 11.2(s, 1H); MS m/z 474(M+H) |
| 33 | 3-pyridin-4-yl-12,13-(2-hydroxy-butan-1,4-yl)-12,13-dihydro-5,7-dioxo-6H- |
| | indolo[2,3-a]pyrrolo[3,4-c]carbazole |
| | ¹H NMR(300MHz, d⁶-DMSO)δ 4.53(m, 5H), 5.45(m, 1H), 7.39(m, 1H), |
| | 7.66(m, 1H), 7.86(d, 2H, J=8Hz), 8.09(m, 3H), 8.70(d, 2H, J=5Hz), 9.19(dd, 1H, |
| | J=8.10Hz), 9.68(dd, 1H, J=2.10Hz), 11.18(s, 1H); MS m/z 473(M+H). |
| 34 | 3-pyrimidin-5-yl-12,13-(2-hydroxy-butan-1,4-yl)-12,13-dihydro-5,7-dioxo-6H- |
| | indolo[2,3-a]pyrrolo[3,4-c]carbazole |
| | ¹H NMR(300MHz, d⁶-DMSO)δ 4.36-4.94(m, 5H), 5.44(m, 1H), 7.39(m, 1H), |
| | 7.64(m, 1H), 7.85(m, 1H), 8.06(m, 2H), 9.17(m, 1H), 9.21(s, 3H), 9.56(m, 1H), |
| | 11.10(s, 1H); MS m/z 474(M+H). |
| 35 | 3-pyrazin-2-yl-12,13-(2-hydroxy-butan-1,4-yl)-12,13-dihydro-5,7-dioxo-6H- |
| | indolo[2,3-a]pyrrolo[3,4-c]carbazole |
| | ¹H NMR(300MHz, d⁶-DMSO)δ 4.37-4.94(m, 5H), 5.47(t, 1H, J=4Hz), |
| | 7.42(q, 1H, J=8Hz), 7.65(q, 1H, J=7Hz), 7.84(m, 1H), 7.99(dd, 1H, J=5.9Hz), |
| | 8.41(m, 1H), 8.61(d, 1H, J=3Hz), 8.78(m, 1H), 9.20(dd, 1H, J=8.16Hz), |
| | 9.31(s, 1H), 9.96(m, 1H), 11.18(s, 1H); MS m/z 473(M+H). |

### BIOLOGICAL EXAMPLES

The ability of the compounds to treat or ameliorate protein kinase mediated disorders was determined using the following procedures.

### Example 1

JAK3 Kinase Assay

JAK3 enzyme activity was assayed relative to enzyme phosphorylation using the method described herein.

Sf21 cells derived from the ovarian tissue of the fall armyworm Spodotera frugiperda were obtained from Pharmingen (San Diego, Calif.) and maintained at a temperature of about 26-28° C. in 1 l Bellco spinner flasks at 60-90 rpm. Cell viability was maintained at 95-100%, as determined by a tryptan blue dye exclusion test.

Sf21 cells were infected with a baculovirus expression vector for JAK3 (JH1 and JH2 domain). After 48 hours of infection, cells were harvested and lysed in Tris-saline (pH 7.6) containing 2% NP-40 and a combination of protease inhibitors (Aprotinin, Pepstatin A, Pefabloc, E-64, Leupeptin, and Benzamidine) on ice for 45 minutes.

The JAK3 enzyme was purified from the lysate using glutathione sepharose beads and the enzyme activity was assessed in Costar flat bottom EIA/RIA 96 well plates. The plates were coated with Neutravidin (110 μL) (Pierce Neutravidin Biotin-binding Protein 31000; 1 mg/mL 1:100) diluted in PBS for 1 hour at room temperature. The plates were washed with PBS-0.1% Tween (3 times) to remove unbound Neutravidin, then 1% BSA (150 NIL) in PBS was added to each well to block non-specific binding. The plates was incubated for 1 hour at room temperature and stored at −80° C. until use.

JAK3 enzyme solution (48 μL) in 1.25×TK buffer (62.5 mM HEPES pH 7.5, 12.5 mM MgCl₂) containing DTT (42 mM) (Sigma, St. Louis, Mo.) was added to each well of a polypropylene 96 well plate.

A test compound (5 μL) diluted in DMSO (48 μL) and biotinylated peptide enzyme substrate (5 μg diluted in TK buffer containing 10 μM ATP) were added to each well using the double dispense feature of a multichannel electronic biohit. Control wells received DMSO vehicle (5 μL). The contents of the wells were mixed for approximately 8 seconds using a multitube vortexer and the reaction mixture was incubated for 1 hour at room temperature.

After incubation, an aliquot of reaction mixture (90 μL) was transferred into a washed Neutravidin coated plate. The plate was incubated for 15 minutes at room temperature and washed 3 times with PBS-T. PY99 anti-phosphotyrosine antibody (100 μL/well) (Santa Cruz #sc-7020HRP diluted 1:6000 in 1× antibody buffer) was added into each well and the plate was incubated for 40 minutes at room temperature. The antibody buffer contained 10% BSA, 100 mM Tris (pH 7.5), 1M NaCl and 1% Tween 20. The plate washed 3 times with PBS-T, then TMB (100 pLXSigma, St. Louis, Mo.) was added to the each well. The plate was incubated for another 40 mins at room temperature in the dark. The reaction was stopped by the addition of 1M H₂SO₄ (50 μL/well) and the optical density was read at 450/650 nm.

Test compounds were assayed in triplicate at 16 concentrations at half-log dilutions starting at 200 μM. A maximum and minimum signal for the assay was determined on each plate. The percent inhibition of a test compound was calculated according to the formula

$$[ \frac{( max signal - test compound ) }{( max signal - min signal )} ] ( 100 ) = \% inhibition$$

For a series of test concentrations, the IC₅₀ was derived by graphing percent inhibition against the log of the concentrations tested for a given compound. The IC₅₀ results are shown in Table 1. For those compounds without an IC₅₀, the percent inhibition results are shown at a test concentration of 1 μM.

TABLE 1
JAK3 IC₅₀ (μM)

| Cpd | IC₅₀ (μM) |
|---|---|
| 1 | 0.022 |
| 2 | 0.04 |
| 3 | 0.007 |
| 4 | 0.072 |
| 5 | 0.039 |
| 6 | 0.171 |
| 7 | 0.005 |
| 8 | 0.008 |
| 9 | 0.003 |
| 10 | 0.016 |
| 11 | 0.009 |
| 12 | 0.001 |
| 13 | 0.009 |
| 14 | 0.007 |
| 15 | 0.653 |
| 16 | 0.105 |
| 17 | 0.045 |
| 18 | 0.032 |
| 19 | 0.053 |
| 20 | 0.012 |
| 21 | 0.012 |
| 22 | 0.072 |
| 23 | 0.022 |
| 24 | 0.037 |
| 25 | 0.029 |
| 26 | 0.095 |
| 27 | 0.015 |
| 28 | 0.058 |
| 29 | 0.014 |
| 30 | 0.068 |
| 31 | 0.008 |
| 32 | 0.035 |
| 33 | 0.040 |
| 34 | 0.066 |
| 35 | 0.013 |
| 36 | 0.030 |
| 37 | 0.004 |
| 38 | >0.400 |
| 39 | 0.031 |
| 40 | >0.400 |
| 41 | 0.008 |
| 42 | 0.149 |
| 43 | 0.112 |
| 44 | 0.122 |
| 45 | 0.061 |
| 46 | 0.066 |
| 47 | 0.030 |
| 48 | 0.011 |
| 49 | 0.039 |
| 50 | 0.031 |
| 51 | >0.400 |
| 52 | 0.446 |
| 53 | >0.400 |
| 54 | 1.00 |
| 55 | 0.314 |
| 56 | 0.115 |
| 57 | 0.041 |
| 58 | 0.098 |
| 59 | 0.547 |
| 60 | 0.156 |
| 61 | 0.543 |
| 62 | 0.496 |
| 63 | 0.129 |
| 64 | 0.036 |
| 65 | 1.00 |
| 66 | 0.040 |
| 67 | 0.247 |
| 68 | 0.433 |
| 69 | 0.055 |
| 70 | 0.071 |
| 71 | 0.038 |
| 72 | 0.012 |
| 73 | 0.042 |
| 74 | 0.014 |
| 75 | 0.035 |
| 76 | 0.064 |
| 77 | 0.086 |
| 78 | 0.179 |
| 79 | 0.048 |
| 80 | 0.100 |
| 81 | 0.434 |
| 82 | 0.134 |
| 83 | 0.029 |
| 84 | 0.187 |
| 85 | 0.014 |
| 86 | 0.033 |
| 87 | 0.169 |
| 88 | 0.113 |
| 89 | 0.027 |
| 90 | 0.144 |
| 91 | 0.044 |
| 92 | 0.097 |
| 93 | 0.065 |
| 94 | 0.516 |
| 95 | 0.159 |
| 96 | 0.295 |
| 97 | 0.175 |
| 98 | 0.108 |
| 99 | 0.184 |
| 100 | 0.173 |
| 101 | 1.100 |
| 102 | 0.081 |
| 103 | 0.436 |
| 104 | 0.127 |
| 105 | 0.015 |
| 106 | 0.417 |
| 107 | 0.180 |
| 108 | 0.046 |
| 109 | >0.200 |
| 110 | 0.916 |
| 111 | 0.460 |
| 112 | 1.08 |
| 113 | 0.047 |
| 114 | 0.125 |
| 115 | >0.200 |
| 116 | >0.200 |
| 117 | 0.015 |
| 118 | 0.036 |
| 119 | 0.015 |
| 120 | 0.017 |
| 121 | 0.054 |
| 122 | 0.063 |
| 123 | 0.080 |
| 124 | 0.052 |
| 125 | 0.027 |
| 126 | 0.055 |
| 127 | 0.048 |
| 128 | 0.094 |
| 129 | 0.067 |
| 130 | 0.048 |
| 131 | 0.078 |
| 132 | 0.028 |
| 133 | 0.054 |
| 134 | 0.053 |
| 135 | >0.200 |
| 136 | 0.123 |
| 137 | 0.175 |
| 138 | 0.161 |
| 139 | 0.160 |
| 140 | 0.072 |
| 141 | 0.117 |
| 142 | >0.200 |
| 143 | ˜0.200 |
| 144 | 0.886 |
| 145 | 0.031 |
| 146 | 0.026 |
| 147 | 0.028 |
| 148 | 0.025 |
| 149 | 0.039 |
| 150 | 0.045 |
| 151 | 0.023 |
| 152 | 0.084 |
| 153 | 0.017 |
| 154 | 0.084 |
| 155 | 0.144 |
| 156 | ˜0.200 |
| 157 | >0.200 |
| 158 | 0.123 |
| 159 | 0.108 |
| 160 | >0.200 |
| 161 | 1.010 |
| 162 | 0.655 |
| 163 | ˜1.00 |
| 164 | 0.377 |
| 165 | 0.278 |
| 166 | 0.578 |
| 167 | 0.202 |
| 168 | 0.346 |
| 169 | 0.073 |
| 170 | 0.202 |
| 171 | 0.677 |
| 172 | 0.120 |
| 173 | 0.069 |
| 174 | 0.166 |
| 175 | 0.244 |
| 176 | 0.177 |
| 177 | 0.143 |
| 178 | 0.154 |
| 179 | 1.06 |
| 180 | 0.706 |
| 181 | 7.36 |
| 182 | 1.67 |
| 183 | 1.27 |
| 184 | 4.36 |
| 185 | 0.069 |
| 186 | 0.178 |
| 187 | 3.00 |
| 188 | 0.034 |
| 189 | 0.003 |
| 190 | 0.044 |
| 191 | 0.115 |
| 192 | 0.007 |
| 193 | 0.135 |
| 194 | 0.503 |
| 195 | 0.284 |
| 196 | ˜1.00 |
| 197 | 0.693 |
| 198 | 0.351 |
| 199 | 0.309 |
| 200 | 0.019 |
| 201 | 0.024 |
| 202 | 0.103 |
| 203 | 0.618 |
| 204 | 0.169 |
| 205 | 0.042 |
| 206 | 0.049 |
| 207 | 0.013 |
| 208 | 3.13 |
| 209 | 0.070 |
| 210 | 1.05 |
| 211 | 0.107 |
| 212 | 0.003 |
| 213 | 0.241 |
| 214 | >50.00 |
| 215 | 0.008 |
| 216 | 0.310 |
| 217 | 0.592 |
| 218 | 0.686 |
| 219 | 0.014 |
| 220 | 0.090 |
| 221 | 0.031 |
| 222 | 0.237 |
| 223 | 0.003 |

While the foregoing specification teaches the principles of the present invention, with examples provided for the purpose of illustration, it will be understood that the practice of the invention encompasses all of the usual variations, adaptations and modifications as come within the scope of the following claims and their equivalents.

Throughout this application, various publications are cited. The disclosure of these publications is hereby incorporated by reference into this application to describe more fully the state of the art to which this invention pertains.

What is claimed is:

1. A compound of formula (I): 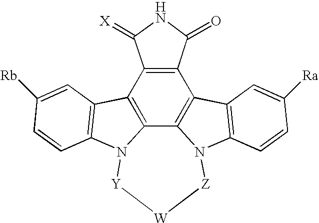
or a form thereof, wherein
X is H, H or O;
Y and Z is each methyl or ethyl;
W is —C(R₁,R₁ₐ)—C(R₂,R₂ₐ)—, —C(R₃)═C(R₄)—, —C(R₅,R₅ₐ)—, —C(R₆)—, —O—, R₇-heterocyclyl, R₇—C₃₋₈cycloalkyl, R₇-heteroaryl or R₇-aryl;
R₁, R₁ₐ, R₂, R₂ₐ, R₅ and R₅ₐ is each selected from R₇, C₁₋₈alkyl-carbamoyl, carbamoyloxy, carbamoyloxy-C₁₋₈alkyl C₁₋₈alkyl-carbamoyloxy, C₁₋₈alkyl-carbamoyloxy-C₁₋₈alkyl, R₇-heterocyclyl-carbamoyl, heterocyclyl-carbonyl, carbonyloxy, heterocyclyl-carbonyloxy or heterocyclyl-carbonyloxy-C₁₋₈alkyl,
wherein when R₁, R₁ₐ, R₂ and R₂ₐ is each selected from R₇, then no more than three of R₁, R₁ₐ, R₂ and R₂ₐ are hydrogen,
wherein when R₅ and R₅ₐ is each selected from R₇, then no more than one of R₅ and R₅ₐ are hydrogen,
wherein C₁₋₈alkyl-carbamoyl is optionally substituted on C₁₋₈alkyl with one, two or three substituents each selected from C₁₋₈alkoxy, C₁₋₈alkoxycarbonyl, amino, C₁₋₈alkyl-amino, halogen, hydroxy, R₇-heterocyclyl, R₇—C₃₋₈cycloalkyl, R₇-heteroaryl or R₇-aryl,
wherein carbamoyloxy and carbamoyloxy-C₁₋₈alkyl is each substituted on nitrogen with one substituent selected from hydrogen or C₁₋₈alkyl and one other substituent selected from R₇-heterocyclyl or R₇-aryl-C₁₋₈alkyl-heterocyclyl,
wherein C₁₋₈alkyl-carbamoyloxy is optionally substituted on C₁₋₈alkyl with one, two or three substituents each selected from C₁₋₈alkoxy, C₁₋₈alkoxycarbonyl, amino, C₁₋₈alkyl-amino, halogen, hydroxy, R₇-heterocyclyl, R₇—C₃₋₈cycloalkyl, R₇-heteroaryl or R₇-aryl,
wherein C₁₋₈alkyl-carbamoyloxy-C₁₋₈alkyl is optionally substituted on C₁₋₈alkyl with one, two or three substituents each selected from C₁₋₈alkoxy, C₁₋₈alkoxycarbonyl, amino, C₁₋₈alkyl-amino, halogen, hydroxy, R₇-heterocyclyl, R₇—C₃₋₈cycloalkyl, R₇-heteroaryl or R₇-aryl,
wherein heterocyclyl-carbonyl is substituted on heterocyclyl with one or two substituents each selected from R₇, R₇—C₃₋₈cycloalkyl, R₇-aryl, R₇-aryl-C₁₋₈alkyl, R₇-heteroaryl, R₇-heteroaryl-C₁₋₈alkyl, R₇-heterocyclyl-C₁₋₈alkyl or R₇-heterocyclyl-carbonyl-C₁₋₈alkyl,
wherein carbonyloxy is substituted on carbonyl with C₁₋₈alkyl, C₁₋₈alkoxy-C₁₋₈alkyl or C₁₋₈alkyl-amino-C₁₋₈alkyl,
wherein heterocyclyl-carbonyloxy is substituted on heterocyclyl with one or two substituents each selected from R₇, R₇—C₃₋₈cycloalkyl, R₇-aryl, R₇-aryl-C₁₋₈alkyl, (R₇-aryl)₂-C₁₋₈alkyl, R₇-heteroaryl, R₇-heteroaryl-C₁₋₈alkyl, R₇-heterocyclyl, R₇-heterocyclyl-C₁₋₈alkyl or R₇-heterocyclyl-C₁₋₈acyl, and
wherein heterocyclyl-carbonyloxy-C₁₋₈alkyl is substituted on heterocyclyl with one or two substituents each selected from R₇, R₇—C₃₋₈cycloalkyl, R₇-aryl, R₇-aryl-C₁₋₈alkyl, (R₇-aryl)₂-C₁₋₈alkyl, R₇-aryl-C₁₋₈alkoxycarbonyl, R₇-heteroaryl, R₇-heteroaryl-C₁₋₈alkyl, R₇-heterocyclyl, R₇-heterocyclyl-C₁₋₈alkyl or R₇-heterocyclyl-C₁₋₈acyl,
alternatively, R₅ and R₅ₐ are taken together with the carbon atom of attachment to form a ring system selected from R₇-heterocyclyl, R₇—C₃₋₈cycloalkyl, R₇-heteroaryl or R₇-aryl, wherein the carbon atom of attachment is a member atom of the ring system;
R₃ and R₄ is each selected from hydrogen, C₁₋₈alkyl, C₁₋₈acyl or C₁₋₈alkoxycarbonyl;
R₆ is selected from C₁₋₈alkylene substituted with one, two or three substituents each selected from C₁₋₈alkoxy, C₁₋₈alkoxycarbonyl, amino, C₁₋₈alkyl-amino, halogen or hydroxy;
R₇ is one, two, three, four or five substituents each selected from hydrogen, C₁₋₈alkyl, C₁₋₈alkoxy, C₁₋₈acyl, amino, C₁₋₈alkyl-amino, C₁₋₈alkyl-amino-C₁₋₈alkyl, carboxy, C₁₋₈alkoxycarbonyl, C₁₋₈alkoxy-amido, halogen, hydroxy, oxo, halo-C₁₋₈alkyl, halo-C₁₋₈alkoxy, hydroxy-C₁₋₈alkyl, hydroxy-C₁₋₈alkoxy, hydroxy-C₁₋₈alkoxy-C₁₋₈alkyl or aminosulfonyl;
Ra and Rb is each selected from R₈, amino-C₁₋₈alkyl, thio-C₁₋₈alkyl, imino-C₁₋₈alkyl, carbamoyl, C₁₋₈alkyl-carbamoyl, C₁₋₈alkyl-carbamoyl-C₂₋₈alkenyl, amino-C₁₋₈alkyl-carbamoyl-C₂₋₈alkenyl, C₁₋₈alkyl-amino-C₁₋₈alkyl-carbamoyl-C₂₋₈alkenyl, R₈-heterocyclyl, R₈-heterocyclyl-C₁₋₈alkyl, R₈-heterocyclyl-C₁₋₈alkoxy, R₈-heterocyclyl-amino, R₈-heterocyclyl-amino-C₂₋₈alkenyl, R₈-heterocyclyl-C₁₋₈acyl-amino, R₈—C₃₋₈cycloalkyl, R₈—C₃₋₈cycloalkyl-C₁₋₈alkyl, R₈-aryl, R₈-aryl-C₁₋₈alkyl, R₈-heteroaryl, R₈-heteroaryl-C₁₋₈alkyl or R₈-heteroaryl-C₂₋₈alkenyl,
wherein amino-C₁₋₈alkyl is optionally substituted on nitrogen with one or two substituents each selected from C₁₋₈alkyl, C₁₋₈alkoxy-C₁₋₈alkyl, R₈-heterocyclyl, R₈-heterocyclyl-C₁₋₈alkyl, R₈—C₃₋₈cycloalkyl-C₁₋₈alkyl, R₈-aryl-C₁₋₈alkyl or R₈-heteroaryl-C₁₋₈alkyl,
wherein thio-C₁₋₈alkyl is substituted on sulfur with C₁₋₈alkyl, amino-C₁₋₈alkyl or C₁₋₈alkyl-amino-C₁₋₈alkyl, and
wherein imino-C₁₋₈alkyl is optionally substituted on nitrogen with C₁₋₈alkyl, C₁₋₈alkoxy-C₁₋₈alkyl, R₈-heterocyclyl-amino, R₈-heterocyclyl-C₁₋₈alkyl, R₈—C₃₋₈cycloalkyl-C₁₋₈alkyl, R₈-aryl-C₁₋₈alkyl, R₈-heteroaryl-amino or R₈-heteroaryl-C₁₋₈alkyl, and
R₈ is one, two, three or four substituents each selected from hydrogen, C₁₋₈alkyl, C₁₋₈alkoxy, C₁₋₈alkoxy-C₁₋₁₈alkyl, C₁₋₈acyl, C₁₋₈alkoxycarbonyl, carboxy, carboxy-C₁₋₈alkyl, carboxy-C₂₋₈alkenyl, amino, C₁₋₈alkyl-amino, halogen, hydroxy, oxo, nitro, halo-C₁₋₈alkyl, halo-C₁₋₈alkoxy, hydroxy-C₁₋₈alkyl or hydroxy-C₁₋₈alkoxy.

2. The compound of claim 1, wherein the compound is an isolated form thereof.

3. The compound of claim 1, wherein the compound is an inhibitor of increased or unregulated JAK3 mediated cytokine expression, signaling or migration.

4. A medicine or medicament comprising one or more of a compound of claim 1.

5. A pharmaceutical composition comprising a compound of claim 1 and a pharmaceutically acceptable carrier.

6. The pharmaceutical composition of claim 5, wherein the effective amount of the compound is in a range of from about 0.001 mg/kg to about 300 mg/kg of body weight per day.

7. A process for preparing a pharmaceutical composition comprising the step of admixing a compound of claim 1 and a pharmaceutically acceptable carrier.

8. A method for treating, preventing or ameliorating a chronic or acute protein kinase mediated disease, disorder or condition in a subject in need thereof comprising administering to the subject an effective amount of a compound of claim 1.

9. The method of claim 8, wherein the kinase is JAK3.

10. The method of claim 8, wherein the disease, disorder or condition is associated with increased or unregulated JAK3 mediated cytokine expression, signaling or migration, whereby such expression, signaling or migration results in an inflammatory response or an immunodeficiency.

11. The method of claim 10, wherein the inflammatory response or immunodeficiency is selected from transplantation rejection, psoriasis, psoriatic arthritis, graft-versus-host disease, multiple sclerosis, inflammatory bowel disease, systemic lupus erythematosus, rheumatoid arthritis, allergic diseases or asthma.

12. The method of claim 8, wherein the effective amount of the compound is in a range of from about 0.001 mg/kg to about 300 mg/kg of body weight per day.

13. The method of claim 8, further comprising administering to the subject an effective amount of a combination product comprising at least one other therapeutic agent in combination with the compound.

14. The method of claim 13, wherein the other agent is an anti-inflammatory agent or an immunosuppressive agent.
